SEQUENCE LISTING (65 sequences)

SEQ ID NO: 1 - protein, 531 aa, Arabidopsis thaliana
MDTLFRLVSLQQQQQSDSIITNQSSLSRTSTTTTGSPQTAYHYNFPQNDVVEECFNFFMDEEDLSSSSSHHNHHNHNNPNTYYSPFTTPTQYHPATSSTPSSTAAAAALASPYSSSGHHNDPSAFSIPQTPPSFDFSANAKWADSVLLEAARAFSDKDTARAQQILWTLNELSSPYGDTEQKLASYFLQALFNRMTGSGERCYRTMVTAAATEKTCSFESTRKTVLKFQEVSPWATFGHVAANGAILEAVDGEAKIHIVDISSTFCTQWPTLLEALATRSDDTPHLRLTTVVVANKFVNDQTASHRMMKEIGNRMEKFARLMGVPFKFNIIHHVGDLSEFDLNELDVKPDEVLAINCVGAMHGIASRGSPRDAVISSFRRLRPRIVTVVEEEADLVGEEEGGFDDEFLRGFGECLRWFRVCFESWEESFPRTSNERLMLERAAGRAIVDLVACEPSDSTERRETARKWSRRMRNSGFGAVGYSDEVADDVRALLRRYKEGVWSMVQCPDAAGIFLCWRDQPVVWASAWRPT

SEQ ID NO: 2 - protein, 653 aa, Arabidopsis thaliana
MAESGDFNGGQPPPHSPLRTTSSGSSSSNNRGPPPPPPPPLVMVRKRLASEMSSNPDYNNSSRPPRRVSHLLDSNYNTVTPQQPPSLTAAATVSSQPNPPLSVCGFSGLPVFPSDRGGRNVMMSVQPMDQDSSSSSASPTVWVDAIIRDLIHSSTSVSIPQLIQNVRDIIFPCNPNLGALLEYRLRSLMLLDPSSSSDPSPQTFEPLYQISNNPSPPQQQQQHQQQQQQHKPPPPPIQQQERENSSTDAPPQPETVTATVPAVQTNTAEALRERKEEIKRQKQDEEGLHLLTLLLQCAEAVSADNLEEANKLLLEISQLSTPYGTSAQRVAAYFSEAMSARLLNSCLGIYAALPSRWMPQTHSLKMVSAFQVFNGISPLVKFSHFTANQAIQEAFEKEDSVHIIDLDIMQGLQWPGLFHILASRPGGPPHVRLTGLGTSMEALQATGKRLSDFADKLGLPFEFCPLAEKVGNLDTERLNVRKREAVAVHWLQHSLYDVTGSDAHTLWLLQRLAPKVVTVVEQDLSHAGSFLGRFVEAIHYYSALFDSLGASYGEESEERHVVEQQLLSKEIRNVLAVGGPSRSGEVKFESWREKMQQCGFKGISLAGNAATQATLLLGMFPSDGYTLVDDNGTLKLGWKDLSLLTASAWTPRS

SEQ ID NO: 3 - protein, 182 aa, Arabidopsis thaliana
MSFSVKGRSLRGNNNGGTGTKCGRWNPTVEQLKILTDLFRAGLRTPTTDQIQKISTELSFYGKIESKNVFYWFQNHKARERQKRRKISIDFDHHHHQPSTRDVFEISEEDCQEEEKVIETLQLFPVNSFEDSNSKVDKMRARGNNQYREYIRETTTTSFSPYSSCGAEMEHPPPLDLRLSFL

SEQ ID NO: 4 - protein, 574 aa, Arabidopsis thaliana
MNSNNWLGFPLSPNNSSLPPHEYNLGLVSDHMDNPFQTQEWNMINPHGGGGDEGGEVPKVADFLGVSKPDENQSNHLVAYNDSDYYFHTNSLMPSVQSNDVVVAACDSNTPNNSSYHELQESAHNLQSLTLSMGTTAGNNVVDKASPSETTGDNASGGALAVVETATPRRALDTFGQRTSIYRGVTRHRWTGRYEAHLWDNSCRREGQSRKGRQVYLGGYDKEDKAARSYDLAALKYWGPSTTTNFPITNYEKEVEEMKHMTRQEFVAAIRRKSSGFSRGASMYRGVTRHHQHGRWQARIGRVAGNKDLYLGTFSTEEEAAEAYDIAAIKFRGLNAVTNFEINRYDVKAILESSTLPIGGGAAKRLKEAQALESSRKREAEMIALGSSFQYGGGSSTGSGSTSSRLQLQPYPLSIQQPLEPFLSLQNNDISHYNNNNAHDSSSFNHHSYIQTQLHLHQQTNNYLQQQSSQNSQQLYNAYLHSNPALLHGLVSTSIVDNNNNNGGSSGSYNTAAFLGNHGIGIGSSSTVGSTEEFPTVKTDYDMPSSDGTGGYSGWTSESVQGSNPGGVFTMWNE

SEQ ID NO: 5 - protein, 568 aa, Arabidopsis thaliana
MNSNNWLAFPLSPTHSSLPPHIHSSQNSHFNLGLVNDNIDNPFQNQGWNMINPHGGGGEGGEVPKVADFLGVSKSGDHHTDHNLVPYNDIHQTNASDYYFQTNSLLPTVVTCASNAPNNYELQESAHNLQSLTLSMGSTGAAAAEVATVKASPAETSADNSSSTTNTSGGAIVEATPRRTLETFGQRTSIYRGVTRHRWTGRYEAHLWDNSCRREGQSRKGRQVYLGGYDKEEKAARAYDLAALKYWGPSTTTNFPITNYEKEVEEMKNMTRQEFVASIRRKSSGFSRGASMYRGVTRHHQHGRWQARIGRVAGNKDLYLGTFSTEEEAAEAYDIAAIKFRGLNAVTNFEINRYDVKAILESNTLPIGGGAAKRLKEAQALESSRKREEMIALGSNFHQYGAASGSSSVASSSRLQLQPYPLSIQQPFEHLHHHQPLLTLQNNNDISQYHDSFSYIQTQLHLHQQQTNNYLQSSSHTSQLYNAYLQSNPGLLHGFVSDNNNTSGFLGNNGIGIGSSSTVGSSAEEEFPAVKVDYDMPPSGGATGYGGWNSGESAQGSNPGGVFTMWNE

SEQ ID NO: 6 - protein, 604 aa, Arabidopsis thaliana
MMAPMTNWLTFSLSPMEMLRSSDQSQFVSYDASSAASSSPYLLDNFYGWSNQKPQEFFKEEAQLAAAASMADSTILTTFVDPQSHHSQNHIPKLEDFLGDSSSIVRYSDNSQTDTQDSSLTQIYDPRHHHNQTGFYSDHHDFKTMAGFQSAFSTNSGSEVDDSASIGRTHLAGDYLGHVVESSGPELGFHGGSTGALSLGVNVNNNTNHRNDNDNHYRGNNNGERINNNNNNDNEKTDSEKEKAVVAVETSDCSNKKIADTFGQRTSIYRGVTRHRWTGRYEAHLWDNSCRREGQARKGRQVFYSFFGMCYLIWGCILALLKINSGYDKEDKAARAYDLAALKYWNATATTNFPITNYSKEVEEMKHMTKQEFIASLRRKSSGFSRGASIYRGVTRHHQQGRWQARIGRVAGNKDLYLGTFATEEEAAEAYDIAAIKFRGINAVTNFEMNRYDVEAIMKSALPIGGAAKRLKLSLEAAASSEQKPILGHHQLHHFQQQQQQQQLQLQSSPNHSSINFALCPNSAVQSQQIIPCGIPFEAAALYHHHQQQQQHQQQQQQQNFFQHFPANAASDSTGSNNNSNVQGTMGLMAPNPAEFFLWPNQSY

SEQ ID NO: 7 - protein, 584 aa, Arabidopsis thaliana
MNSMNNWLGFSLSPHDQNHHRTDVDSSTTRTAVDVAGGYCFDLAAPSDESSAVQTSFLSPFGVTLEAFTRDNNSHSRDWDINGGACNNINNNEQNGPKLENFLGRTTTIYNTNETVVDGNGDCGGGDGGGGGSLGLSMIKTWLSNHSVANANHQDNGNGARGLSLSMNSSTSDSNNYNNNDDVVQEKTIVDVVETTPKKTIESFGQRTSIYRGVTRHRWTGRYEAHLWDNSCKREGQTRKGRQVYLGGYDKEEKAARAYDLAALKYWGTTTTTNFPLSEYEKEVEEMKHMTRQEYVASLRRKSSGFSRGASIYRGVTRHHQHGRWQARIGRVAGNKDLYLGTFGTQEEAAEAYDIAAIKFRGLSAVTNFDMNRYNVKAILESPSLPIGSSAKRLKDVNNPVPAMMISNNVSESANNVSGWQNTAFQHHQGMDLSLLQQQQERYVGYYNGGNLSTESTRVCFKQEEEQQHFLRNSPSHMTNVDHHSSTSDDSVTVCGNVVSYGGYQGFAIPVGTSVNYDPFTAAEIAYNARNHYYYAQHQQQQQIQQSPGGDFPVAISNNHSSNMYFHGEGGGEGAPTFSVWNDT

SEQ ID NO: 8 - protein, 558 aa, Arabidopsis thaliana
MKNNNNKSSSSSSYDSSLSPSSSSSSHQNWLSFSLSNNNNNFNSSSNPNLTSSTSDHHHPHPSHLSLFQAFSTSPVERQDGSPGVSPSDATAVLSVYPGGPKLENFLGGGASTTTTRPMQQVQSLGGVVFSSDLQPPLHPPSAAEIYDSELKSIAASFLGNYSGGHSSEVSSVHKQQPNPLAVSEASPTPKKNVESFGQRTSIYRGVTRHRWTGRYEAHLWDNSCRREGQSRKGRQVYLGGYDKEDKAARAYDLAALKYWGPTTTTNFPISNYESELEEMKHMTRQEFVASLRRKSSGFSRGASMYRGVTRHHQHGRWQARIGRVAGNKDLYLGTFSTQEEAAEAYDIAAIKFRGLNAVTNFDISRYDVKSIASCNLPVGGLMPKPSPATAAADKTVDLSPSDSPSLTTPSLTFNVATPVNDHGGTFYHTGIPIKPDPADHYWSNIFGFQANPKAEMRPLANFGSDLHNPSPGYAIMPVMQEGENNFGGSFVGSDGYNNHSAASNPVSAIPLSSTTTMSNGNEGYGGNINWINNNISSSYQTAKSNLSVLHTPVFGLE

SEQ ID NO: 9 - DNA, 1596 bp, Arabidopsis thaliana
atggatactctctttagactagtcagtctccaacaacaacaacaatccgatagtatcattacaaatcaatcttcgttaagcagaacttccaccaccactactggctctccacaaactgcttatcactacaactttccacaaaacgacgtcgtcgaagaatgcttcaactttttcatggatgaagaagacctttcctcttcttcttctcaccacaaccatcacaaccacaacaatcctaatacttactactctcctttcactactcccacccaataccatcccgccacatcatcaaccccttcctccaccgccgcagccgcagctttagcctcgccttactcctcctccggccaccataatgacccttccgcgttctccatacctcaaactcctccgtccttcgacttctcagccaatgccaagtgggcagactcggtccttcttgaagcggcacgtgccttctccgacaaagacactgcacgtgcgcaacaaatcctatggacgctcaacgagctctcttctccgtacggagacaccgagcaaaaactggcttcttacttcctccaagctctcttcaaccgcatgaccggttcaggcgaacgatgctaccgaaccatggtaacagctgcagccacagagaagacttgctccttcgagtcaacgcgaaaaactgtactaaagttccaagaagttagcccctgggccacgtttggacacgtggcggcaaacggagcaatcttggaagcagtagacggagaggcaaagatccacatcgttgacataagctccacgttttgcactcaatggccgactcttctagaagctttagccacaagatcagacgacacgcctcacctaaggctaaccacagttgtcgtggccaacaagtttgtcaacgatcaaacggcgtcgcatcggatgatgaaagagatcggaaaccgaatggagaaattcgctaggcttatgggagttcctttcaaatttaacattattcatcacgttggagatttatctgagtttgatctcaacgaactcgacgttaaaccagacgaagtcttggccattaactgcgtaggcgcgatgcatgggatcgcttcacgtggaagccctagagacgctgtgatatcgagtttccgacggttaagaccgaggattgtgacggtcgtagaagaagaagctgatcttgtcggagaagaagaaggtggctttgatgatgagttcttgagagggtttggagaatgtttacgatggtttagggtttgcttcgagtcatgggaagagagttttccaaggacgagcaacgagaggttgatgctagagcgtgcagcgggacgtgcgatcgttgatcttgtggcttgtgagccgtcggattccacggagaggcgagagacagcgaggaagtggtcgaggaggatgaggaatagtgggtttggagcggtggggtatagtgatgaggtggcggatgatgtcagagctttgttgaggagatataaagaaggtgtttggtcgatggtacagtgtcctgatgccgccggaatattcctttgttggagagatcagccggtggtttgggctagtgcgtggcggccaacgtaa

SEQ ID NO: 10 - DNA, 1962 bp, Arabidopsis thaliana
atggcggaatccggcgatttcaacggtggtcaacctcctcctcatagtcctctgagaacaacttcttccggtagtagcagcagcaacaaccgtggtcctcctcctcctcctcctcctcctttagtgatggtgagaaaaagattagcttccgagatgtcttctaaccctgactacaacaactcctctcgtcctcctcgccgtgtctctcaccttcttgactccaactacaatactgtcacaccacaacaaccaccgtctcttacggcggcggctactgtatcttctcaaccaaacccaccactctctgtttgtggcttctctggtcttcccgtttttccttcagaccgtggtggtcggaatgttatgatgtccgtacaaccaatggatcaagactcttcatcttcttctgcttcacctactgtatgggttgacgccattatcagagaccttatccattcctcaacttcagtctctattcctcaacttatccaaaacgttagagacattatcttcccttgtaacccaaatctcggtgctcttcttgaatacaggctccgatctctcatgctccttgatccttcctcttcctctgacccttctcctcaaactttcgaacctctctatcagatctccaacaatccttctcctccacaacagcaacagcagcaccaacaacaacaacaacagcataagcctcctcctcctccgattcagcagcaagaaagagaaaattcttctaccgatgcaccaccgcaaccagagacagtgacggccactgttcccgccgtccaaacaaatacggcggaggctttaagagagaggaaggaagagattaagaggcagaagcaagacgaagaaggattacaccttctcacattgctgctacagtgtgctgaagctgtctctgctgataatctcgaagaagcaaacaagcttcttcttgagatctctcagttatcaactccttacgggacctcagcgcagagagtagctgcttacttctcggaagctatgtcagcgagattactcaactcgtgtctcggaatttacgcggctttgccttcacggtggatgcctcaaacgcatagcttgaaaatggtctctgcgtttcaggtctttaatgggataagccctttagtgaaattctcacactttacagcgaatcaggcgattcaagaagcatttgagaaagaagacagtgtacacatcattgacttggacatcatgcagggacttcaatggcctggtttattccacattcttgcttctagacctggaggacctccacacgtgcgactcacgggacttggtacttccatggaagctcttcaggctacagggaaacgtctttcggatttcgcagataagcttggcctgccttttgagttctgccctttagctgagaaagttggaaacttggacactgagagactcaatgtgaggaaaagggaagctgtggctgttcactggcttcaacattctctttatgatgtcactggctctgatgcacacactctctggttactccaaagattagctcctaaagttgtgacagtagtggaacaagatttgagccacgctggttctttcttaggaagatttgtagaagcaatacattactactctgcactctttgactcactgggagcaagctacggcgaagagagtgaagagagacatgtcgtggaacagcagctattatcgaaagagatacggaatgtattagcggttggaggaccatcgagaagcggtgaagtgaagtttgagagctggagggagaaaatgcaacaatgtgggtttaaaggtatatctttagctggaaatgcagctacacaagcgactctactgttgggaatgtttccttcggatggttacactttggttgatgataatggtacacttaagcttggatggaaagatctttcgttactcactgcttcagcttggacgcctcgttcttag

SEQ ID NO: 11 - DNA, 549 bp, Arabidopsis thaliana
atgtctttctccgtgaaaggtcgaagcttacgtggcaacaataacggaggaacggggacgaagtgcgggagatggaatccaacggtggagcagttgaagatattgactgatctgtttcgagccggtcttagaactccaacaactgatcagattcagaagatctctacggagctcagtttctacggcaagatagagagcaagaatgttttctattggtttcagaatcataaggctagggagaggcagaaacgtcgtaaaatctccattgattttgatcatcatcatcatcaaccatcaactagagatgtttttgaaataagcgaagaagattgtcaagaggaagagaaggtgatagagacattacaactctttccggtgaattcatttgaagactccaactccaaggtggacaaaatgagagctagaggcaataaccagtaccgtgaatatattcgagagaccaccacgacgtcgttttctccatactcatcatgtggagctgaaatggaacatccaccgccattagatcttcgattaagctttctttaa

SEQ ID NO: 12 - DNA, 1725 bp, Arabidopsis thaliana
atgaattctaacaactggcttggctttcctctttcaccgaacaactcttctttgcctcctcatgaatacaaccttggcttggtcagcgaccatatggacaacccttttcaaacacaagagtggaatatgatcaatccacacggtggaggaggagatgaaggaggagaggttccaaaagtggccgattttctcggtgtgagcaaaccggacgaaaaccaatccaaccacctagtagcttacaacgactcagactactacttccataccaatagcttgatgcctagcgtccaatcaaacgatgtcgttgtagcagcttgtgactccaatactcctaacaacagtagctatcatgagcttcaagagagtgctcacaatctacagtcacttactttgtccatggggaccaccgctggtaataatgttgtagacaaagcttcaccatccgagaccaccggggataacgctagcggtggagcactagccgttgttgagacggccacgccaagacgtgcattggacactttcggacaacgaacctcgatctatcgtggtgtcacaagacatcgatggactggtcgatatgaggctcatctatgggataatagttgtagaagggaaggccagtctaggaaaggaagacaagtttacttgggtggatatgacaaagaagataaagcagcaagatcatatgatctagctgcacttaagtactggggtccttcaactactactaatttccccattacaaactacgagaaagaagtagaggaaatgaagcacatgacgagacaagagttcgtggctgccattagaaggaaaagtagtggattttcgagaggcgcttcgatgtatcgaggagttacaaggcatcaccaacatggaagatggcaagcaaggatcggccgagtcgccggaaacaaagacctctacttgggaacttttagcactgaggaagaagcagcagaagcttacgatatagctgcaataaagtttagaggacttaatgcagtgaccaacttcgagatcaaccggtacgacgtgaaagccattctagagagtagcactcttcccatcggaggaggcgcagctaaacggctcaaagaagctcaagctcttgagtcttcaaggaaacgcgaggcggagatgatagcccttggttcaagtttccagtacggtggtggctcgagcacaggctctggctccacctcatcaagacttcagcttcaaccttaccctctaagcattcaacaaccattagagccttttctatctcttcagaacaatgacatctctcattacaacaacaacaatgctcacgattcctcctcttttaatcaccatagctatatccagacacaacttcatctccaccaacagaccaacaattacttgcagcaacagtcgagccagaactctcagcagctctacaatgcgtatcttcatagcaatccggctctgcttcatggacttgtctctacctctatcgttgacaacaataataacaatggaggctctagtgggagctacaacactgcagcatttcttgggaaccacggtattggtattgggtccagctcgactgttggatcgaccgaggagtttccaaccgttaaaacagattacgatatgccttccagtgatggaaccggagggtatagtggttggaccagtgagtctgttcaggggtcaaaccctggtggtgttttcactatgtggaatgagtaa

SEQ ID NO: 13 - DNA, 1707 bp, Arabidopsis thaliana
atgaattctaacaactggctcgcgttccctctatcaccaactcactcttctttgccgcctcacattcactcttcacaaaattctcatttcaatctaggtttggtcaacgacaatatcgacaacccttttcaaaaccaaggatggaatatgatcaatccacatggtggaggcggcgaaggtggagaggttccaaaagtggctgatttcttaggagtgagcaaatcgggggatcatcacaccgatcacaacctcgtaccttataacgacattcatcaaaccaacgcctccgactactactttcaaaccaatagcttgttacctacagtcgtcacttgtgcctctaatgctcctaataattatgagcttcaagagagtgcacacaatttgcaatctctcactctctctatgggaagtactggagctgccgctgcagaagtcgccactgtgaaagcctcgccggctgagactagtgccgataatagtagcagcactaccaacacaagtggaggagccatcgttgaggctacaccgagacggactttggaaacttttggacaacgaacctctatctatcgtggagttacaagacatagatggaccggtagatatgaagctcatctttgggataatagctgtagaagagaaggacaatcaaggaaaggaagacaagtctacttaggtgggtatgacaaagaagagaaagcagccagagcatatgatctagctgcacttaaatattggggtccctctactactaccaactttccgataactaactacgagaaggaagtagaggagatgaaaaacatgacgagacaagagtttgtggcttctataagaaggaaaagtagcggattctcgcgtggtgcatccatgtatcgtggagtaacaaggcatcatcaacatggaagatggcaagcaaggatcggccgagttgctggaaacaaagatctctacttgggaacattcagcacggaggaagaagcagcagaagcttatgacatagctgcgataaagtttcgaggtctaaacgcggttacaaactttgagataaatcggtatgatgtgaaagccatcctggagagcaacacacttcctataggaggtggtgcggctaaacggctcaaagaagctcaagctctagaatcatcaagaaaacgagaggaaatgatagccctcggatcaaatttccatcaatatggtgcagcgagcggctcgagctctgttgcttccagctctaggcttcagcttcaaccttaccctctaagcattcaacaaccttttgagcatcttcatcatcatcagcctttacttactctacagaacaacaacgatatctctcagtatcatgattcctttagttacattcagacgcagcttcatcttcaccaacaacaaaccaacaattacttgcagtcttctagtcacacttcacagctctacaatgcttatcttcagagtaaccctggtctgcttcatggatttgtctctgataataacaacacttcagggtttcttggaaacaatgggattggtattgggtcaagctctaccgttggatcatcggctgaggaagagtttccagccgtgaaagtcgattacgatatgcctccttccggtggagctacagggtatggaggatggaatagtggagagtctgctcaaggatcgaatccaggaggtgttttcacgatgtggaatgaataa

SEQ ID NO: 14 - DNA, 1815 bp, Arabidopsis thaliana
atgatggctccgatgacgaactggttaacgttttctctgtcaccaatggagatgttgaggtcatctgatcagtctcagtttgtctcctatgacgcttcttccgccgcttcctcctctccttatctcctcgataatttctatggttggtcaaaccaaaaacctcaggagtttttcaaagaagaagctcagttagcagcagcagcttcaatggcggattcaacaatcttaacaacattcgtagacccacaatctcatcattctcagaatcacatcccaaagctcgaagattttctcggtgactcttcttctatcgttcgttactccgacaacagtcaaaccgacacacaagactcttccctcactcaaatctacgatccacgtcaccaccataaccaaaccggcttttactccgatcaccacgatttcaaaaccatggccggttttcaatccgctttctctactaactccggttcagaggtcgatgactctgcttctatcggaaggactcatcttgctggagactatttgggacacgtggttgaatcttctggtccggagctagggtttcacggtggatctaccggagctttgtcacttggtgttaacgtcaataacaatactaatcaccggaatgataatgataatcattaccgaggcaataacaatggtgagagaatcaacaacaacaacaacaatgacaacgagaagacagattctgagaaggagaaggctgttgtggctgtggaaacatcagattgttctaataagaagattgctgatacgtttggtcaaaggacttcgatttacagaggtgttactcgacatagatggacgggaagatatgaagcacatctatgggataatagctgtaggagagaaggtcaggccaggaagggacgtcaagtcttctactctttttttggcatgtgctacttgatatggggatgcattttggctctgcttaaaatcaacagtggatatgacaaagaagataaggcagctcgagcttacgatttagcagctctgaaatactggaatgctactgctaccaccaatttccctattacgaattactcgaaagaagtggaggaaatgaagcacatgaccaagcaagagttcattgcctccctcaggaggaagagtagcggtttctctagaggagcttcgatataccgaggtgttacaaggcatcatcaacaaggacgttggcaagcaaggattggccgagttgctgggaacaaagatctttaccttggaacctttgcaacggaagaggaagcagctgaagcgtatgacatagcagcaatcaaattcagaggaataaacgctgtaactaactttgagatgaaccgttacgacgttgaagccatcatgaagagtgcacttcccatcggtggtgcagctaaacgtcttaagctctctttggaagctgctgcttcatcagagcagaaaccaatcctcggtcatcatcaacttcaccatttccagcaacaacaacaacaacaacagcttcagcttcagtcatctcctaatcacagtagcattaacttcgctctctgtcctaattcagcagttcagtctcaacagatcattccttgtggaatcccttttgaagcagctgctctttaccaccaccaccaacaacaacagcaacaccaacagcagcagcagcaacagaacttcttccagcattttccggcgaatgcagcttctgactcgaccgggtctaacaacaactccaacgttcagggaacaatgggacttatggcaccaaatccggctgagttcttcctctggcctaatcagtcttactaa

SEQ ID NO: 15 - DNA, 1755 bp, Arabidopsis thaliana
atgaactcgatgaataactggttaggcttctctctctctcctcatgatcaaaatcatcaccgtacggatgttgactcctccaccaccagaaccgccgtagatgttgccggagggtactgttttgatctggccgctccctccgatgaatcttctgccgttcaaacatcttttctttctcctttcggtgtcaccctcgaagctttcaccagagacaataatagtcactcccgagattgggacatcaatggtggtgcatgcaataacattaacaataacgaacaaaatggaccaaagcttgagaatttcctcggccgcaccaccacgatttacaataccaacgagaccgttgtagatggaaatggcgattgtggaggaggagacggtggtggtggcggctcactaggcctttcgatgataaaaacatggctgagtaatcattcggttgctaatgctaatcatcaagacaatggtaacggtgcacgaggcttgtccctctctatgaattcatctactagtgatagcaacaactacaacaacaatgatgatgtcgtccaagagaagactattgttgatgtcgtagaaactacaccgaagaaaactattgagagttttggacaaaggacgtctatataccgcggtgttacaaggcatcggtggacaggtagatacgaggcacatttatgggacaatagttgcaaaagagaaggccagactcgcaaaggaagacaagtttatctgggaggttatgacaaagaagaaaaagcagctagggcttacgatttagccgcactaaagtattggggaaccaccactactactaacttccccttgagtgaatatgagaaagaggtagaagagatgaagcacatgacgaggcaagagtatgttgcctctctgcgcaggaaaagtagtggtttctctcgtggtgcatcgatttatcgaggagtaacaaggcatcaccaacatggaaggtggcaagctaggatcggaagagtcgccggtaacaaagacctctacttgggaactttcggcacacaggaagaggctgctgaggcttatgacattgcagccattaaattcagaggattaagcgcagtgactaacttcgacatgaacagatacaatgttaaagcaatcctcgagagcccgagtctacctattggtagttctgcgaaacgtctcaaggacgttaataatccggttccagctatgatgattagtaataacgtttcagagagtgcaaataatgttagcggttggcaaaacactgcgtttcagcatcatcagggaatggatttgagcttattgcagcaacagcaggagaggtacgttggttattacaatggaggaaacttgtctaccgagagtactagggtttgtttcaaacaagaggaggaacaacaacacttcttgagaaactcgccgagtcacatgactaatgttgatcatcatagctcgacctctgatgattctgttaccgtttgtggaaatgttgttagttatggtggttatcaaggattcgcaatccctgttggaacatcggttaattacgatccctttactgctgctgagattgcttacaacgcaagaaatcattattactatgctcagcatcagcaacaacagcagattcagcagtcgccgggaggagattttccggtggcgatttcgaataaccatagctctaacatgtactttcacggggaaggtggtggagaaggggctccaacgttttcagtttggaacgacacttag

SEQ ID NO: 16 - DNA, 1677 bp, Arabidopsis thaliana
atgaagaacaataacaacaaatcttcttcttcttctagctatgattcttctttgtctccttcttcttcatcctcctcccaccagaactggctctctttctctctctccaacaataacaacaacttcaattcttcctcaaaccctaatctcacttcctccacatcagatcatcatcatcctcacccttctcacctctctctctttcaagctttctccacttctccagtcgaacggcaagatgggtcaccgggagtttcacccagcgatgccacggcggttctttccgtataccccggcggtcctaaacttgagaacttcctcggcggaggagcctcaacgacgacaacaagaccaatgcaacaagtgcaatctcttggcggcgttgtcttctcttccgacctacagccaccgcttcatcctccgtccgccgccgagatctacgactctgagctcaagtcaatagccgctagcttcctaggaaactactccggtggacactcgtcggaggtctctagcgtacataaacaacaaccgaatcctctagctgtctcagaggcttcgcctactccgaagaagaacgtagagagttttggacaacgtacctcgatttatagaggagtcacaagacatagatggactggaagatacgaagctcatctatgggataatagttgccgaagagaaggccaaagcagaaaaggaagacaagtttatttaggtggttatgataaggaagataaagcagctagagcttacgaccttgcagctcttaagtattggggtcctacaactacgactaatttcccgatatcaaattacgaatctgaacttgaagaaatgaaacacatgactcgacaagagttcgttgcttctttaagacggaaaagcagtggattctctaggggtgcctccatgtacagaggcgtcactagacatcatcagcatggtcgatggcaggcacgaattggaagagttgcaggcaacaaagacctttatcttggcacatttagcactcaagaggaagctgcagaagcttatgatatagcagcgatcaaattccgcggtctaaatgcagtcaccaatttcgacatcagtcgatatgatgtcaaatcaattgctagctgtaatctccctgtgggtggactaatgcctaaaccttctccagcaaccgcagcggctgacaaaaccgttgatctttctccatccgactctccatctctaaccacaccgtccctcacgttcaatgtggcaacaccggtcaatgaccatggaggaactttttaccacactggtataccaatcaaaccagacccggctgatcattattggtccaacatctttggattccaggcaaacccgaaagcagaaatgcgaccattagcaaactttgggtcggatcttcataacccttctcctggttatgctataatgccggtaatgcaggaaggtgaaaacaactttggtggtagttttgttgggtctgatgggtataacaatcattccgctgcatcgaacccggtctcagcaattccgctgtcctcgacaactacaatgagtaacggtaacgaagggtatggtggaaacataaactggattaataacaacatttcaagttcttaccaaactgcaaaatcaaatctctctgttttgcacacaccggtttttgggttggaatga

SEQ ID NO: 17 - protein, 1013 aa, Arabidopsis thaliana
MEEVQPPVTPPIEPNGKRSEASLLDICEKVLSLDGSTCDEALKLFTETKRILSASMSNIGSGTREEVERFWFAFILYSVKRLSVRKEADGLSVSGDNEFNLCQILRALKLNIVDFFKELPQFVVKAGSVLGELYGADWENRLQAKEVQANFVHLSLLSKYYKRGFREFFLTYDANAEKNSANSSTYLLDSYRFGWLLFLALRNHAFSRFKDLVTCSNGVVSILAILIIHVPCRFRNFSIQDSSRFVKKGDKGVDLVASLCKIYDASEDELRIVIDKANNLVETILKKKPSPASECQTDKLDNIDPDGLTYFEDLLEETSISTSLITLEKDYYDGKGELDERVFINEEDSLLGSGSLSAGAVNITGVKRKIDALSSPARTFISPLSPHKSPAAKTNGISGATKLAATPVSTAMTTAKWLRTVISPLLPKPSPGLEHFLKSCDRDITNDVTRRAHIILEAIFPNSSLGAQCGGGSLQAVDLMDDIWAEQRRLEACKLYYRVLEAMCKAEAQILHANNLNSLLTNERFHRCMLACSAELVLATHKTITMLFPAVLERTGITAFDLSKVIESFIRHEDSLPRELRRHLNSLEERLLESMVWEKGSSMYNSLIVARPSLALEINQLGLLAEPMPSLDAIAALINFSDGANHASSVQKHETCPGQNGGIRSPKRLCTDYRSILVERNSFTSPVKDRLLALGNVKSKMLPPPLQSAFASPTRPNPGGGGETCAETGINIFFTKINKLAAVRINGMVERLQLSQQIRESVYCFFQHVLAQRTSLLFSRHIDQIILCCFYGVAKISQMSLTFREIIYNYRKQPQCKPLVFRSVYVDALQCRRQGRIGPDHVDIITFYNEIFIPAVKPLLVELGPVRNDRAVEANNKPEGQCPGSPKVSVFPSVPDMSPKKVSAVHNVYVSPLRGSKMDALISHSTKSYYACVGESTHAYQSPSKDLSAINNRLNNSSSNRKRTLNFDAEAGMVSDSMVANSLNLQNQNQNQNGSDASSSGGAAPLKTEPTDS

SEQ ID NO: 18 - DNA, 3042 bp, Arabidopsis thaliana
atggaagaagttcagcctccagtgaccccgcccattgaaccaaatgggaaaagaagcgaagcctctctcttggacatatgcgagaaagttctgtctcttgatgggagcacttgcgatgaagctttgaagttgtttacagaaaccaaacgaattttgtcagcaagcatgtctaacattggaagtggaacgcgggaagaagtagagaggttctggtttgcgtttattctctattcagtgaagaggcttagtgtgagaaaagaagcggatggtctgtcagtgtctggtgataatgagtttaatctatgtcagatactgagggctctgaagctaaatattgtggatttttttaaagagttacctcagtttgtggtcaaggctggatctgtactgggtgaactttacggcgcagactgggagaacagacttcaggcaaaggaggtgcaggctaactttgtgcatcttagccttctaagcaaatactacaaacgtgggttccgggaattctttttgacatatgatgcaaacgcagaaaagaactcagcaaactcttctacctatttgctggatagttatcgttttggatggctactctttttggcactccgaaaccatgcgtttagtcgatttaaggacctcgtgacatgctcaaatggcgtagtttctatattggctattttgatcatacatgttccttgtcggtttagaaatttcagcatccaagattcttctcgctttgttaagaaaggtgacaaaggtgtagacttggttgcatcactttgcaagatatatgacgcctcagaagatgagttgaggatagtaattgacaaggcaaataatttggtagaaaccatactgaagaaaaagccatctccagcatctgagtgccaaactgacaagctagataatattgacccagatggcttgacctactttgaggatttactggaagagacgtccatctcaactagcttaattacacttgaaaaggattactatgatggtaaaggcgaacttgatgagagggtattcatcaatgaagaggatagcttacttggatctggaagcttatctgcaggagctgttaatattactggtgttaagaggaaaattgatgctttgagctcacctgcaaggacatttataagcccactttctcctcataagtcgcctgctgctaagacaaatggtattagcggtgctaccaagttggcagcaacaccagtgagcacagcaatgacaactgccaagtggctcaggactgtcatatccccgcttctgccaaaaccttctcctgggttggaacatttccttaaatcatgtgatagggatataacaaatgacgtcacacgaagagcacacataatattggaagctattttcccaaatagttcccttggtgcccaatgtggaggtggaagtttgcaagctgttgacctgatggatgacatatgggcagagcagcgcagattagaagcttgtaagttatactacagagttcttgaggcaatgtgtaaagcagaagctcagattttgcatgcaaataatctgaactctttattgacaaatgagaggttccatagatgcatgcttgcttgctcagctgaattggtactggctacccacaaaacaattacaatgttgttcccagctgttctggagaggactgggatcacagcctttgatctcagcaaggtaattgagagtttcatacgacatgaagattctctgcctagagagttgagacgacatctgaattcactggaggaacggcttctagagagtatggtatgggagaaaggctcttcaatgtacaattctctgattgttgccaggccatcgcttgcattggagataaatcagctcggtttactagctgaaccaatgccatctctggatgcaatcgcagcacttattaatttctctgacggagcaaatcatgcatcatctgtacaaaagcatgaaacttgtccaggacaaaatggggggattagatcgcccaaaagattatgtactgattaccgcagcattctagttgaacgcaattcctttacatcaccagtaaaggatcgtctgttggccttaggcaacgttaaatccaagatgctgccacctccgttgcagtctgcatttgccagcccaacacggcccaacccaggaggtggaggagaaacttgtgcagaaactggaatcaatattttcttcacaaagattaataaattggctgctgtaagaatcaatggaatggtggaaagactacaactttcacagcaaataagggagagtgtgtattgtttcttccaacatgtacttgctcagcggacttctcttttattcagtcgacacattgaccagatcattctctgttgcttctacggagtggccaagatatcccaaatgagcctgactttcagggaaatcatatacaactaccggaagcaaccacagtgtaaaccattagttttccgcagcgtttatgtggatgcgttacaatgtcgccgtcaagggagaatagggccagatcatgttgacatcatcacattctacaatgaaatatttattcctgccgtaaagccgctgctggtggagctaggtcctgtaagaaacgaccgggctgtggaagccaataataagcctgaaggtcaatgtcccggatcgccaaaggtgtctgtgtttccaagtgttccagacatgtcccctaaaaaagtatctgcagtgcacaatgtttatgtttctcctcttcggggatcaaagatggatgctcttatttcacacagtacaaagagttactatgcttgtgttggagagagtacacatgcttaccagagcccttcaaaggacctatctgccatcaacaaccgcttgaacaacagcagcagcaaccgcaagaggacgctaaactttgacgcagaagcagggatggtcagcgattccatggtagcaaatagccttaacctccaaaaccaaaatcaaaaccaaaatggaagcgatgcatcgtcctcaggtggtgccgcaccccttaaaaccgagccaacagattcatag

SEQ ID NO: 19 - DNA, 1497 bp, Arabidopsis thaliana
atggctcctccaatgacgaattgcttaacgttttctctgtcaccaatggagatgttgaaatcaactgatcagtctcacttctcttcttcttacgacgattcttctactccttatctcatcgataacttctatgctttcaaagaagaagctgagatagaagctgctgctgcttcaatggcggattcaacaaccttatctacttttttcgatcattctcagactcagattccaaagctggaagatttcctcggtgattcctttgtccgttactctgataaccaaacagagacccaagactcttcttctctcactccattctacgatccacgtcaccgcaccgttgccgaaggagttacagggttcttctctgatcatcatcagccagatttcaagacgataaactcgggaccagaaatcttcgatgactcaacaacttccaacatcggtggtactcatctctccagtcacgtggtggagtcatcaacgacggcgaagttagggtttaacggtgattgcaccaccaccggaggagttttgtctctaggggttaacaacacatcagatcaacctttgagctgtaacaatggcgagagaggtggaaacagtaacaagaagaaaacagtttctaagaaggaaacatcagatgattcaaagaagaagattgtcgaaacattgggacaaagaacttcaatttatcgtggagtcacccgacatagatggactggaagatacgaagcgcatctatgggataacagctgtaggagggaaggtcaagccagaaaaggacgtcaagtgtacttaggtggatatgacaaggaagatagagcagctagagcctatgacttggcagctttaaaatactggggttctactgctactacaaattttccggtctcgagttattcaaaagaacttgaggaaatgaatcacatgaccaagcaagagtttattgcatctcttaggaggaaaagtagcggtttttcgagaggagcttcaatatatagaggtgtcacaaggcatcatcaacaaggtcgctggcaagcaagaatcggccgtgtcgcaggaaacaaagatctttacctcggaacctttgcaaccgaagaggaagcagcagaggcttatgacattgcagccataaagttcagaggaatcaacgcagtaactaactttgagatgaacaggtatgacattgaagctgtcatgaatagttctttacctgtaggaggagcagctgcgaaacgccacaaactcaaactcgctcttgaatctccttcttcatcatcctctgaccataacctccaacaacaacagttgcttccgtcctcttctccctcggatcaaaaccctaactcaatcccatgtggcattccatttgagccttcagttctctattaccaccagaacttctttcagcattatcctttggtctctgactctacaattcaagctcctatgaaccaagctgagtttttcttgtggcctaaccagtcttactaa

SEQ ID NO: 20 - DNA, 1519 bp, Artificial Sequence
  GVG
aatgaagctcctgtcctccatcgagcaggcctgcgacatctgccgcctcaagaagctcaagtgctccaagaagaagccgaagtgcgccaagtgtctgaagaacaactgggagtgtcgctactctcccaaaaccaagcgctccccgctgacccgcgcccacctcaccgaagtggagtcccgcctggagcgcctggagcagctcttcctcctgatcttccctcgagaggacctcgacatgatcctgaaaatggactccctccaggacatcaaagccctgctcaccggcctcttcgtccaggacaacgtgaacaaagacgccgtcaccgaccgcctggcctccgtggagactgacatgcccctcaccctgcgccagcaccgcatcagcgcgacctcctcctcggaggagagcagcaacaagggccagcgccagttgaccgtctcgacggcccccccgaccgacgtcagcctgggggacgagctccacttagacggcgaggacgtggcgatggcgcatgccgacgcgctagacgatttcgatctggacatgttgggggacggggattccccgggtccgggatttaccccccacgactccgccccctacggcgctctggatatggccgacttcgagtttgagcagatgtttaccgatgcccttggaattgacgagtacggtggggatccaattcagcaagccactgcaggagtctcacaagacacttcggaaaatcctaacaaaacaatagttcctgctgcattaccacagctcacccctaccttggtgtcactgctggaggtgattgaacccgaggtgttgtatgcaggatatgatagctctgttccagattcagcatggagaattatgaccacactcaacatgttaggtgggcgtcaagtgattgcagcagtgaaatgggcaaaggcgataccaggcttcagaaacttacacctggatgaccaaatgaccctgctacagtactcatggatgtttctcatggcatttgccctgggttggagatcatacagacaatcaagtggaaacctgctctgctttgctcctgatctgattattaatgagcagagaatgtctctaccctgcatgtatgaccaatgtaaacacatgctgtttgtctcctctgaattacaaagattgcaggtatcctatgaagagtatctctgtatgaaaaccttactgcttctctcctcagttcctaaggaaggtctgaagagccaagagttatttgatgagattcgaatgacttatatcaaagagctaggaaaagccatcgtcaaaagggaagggaactccagtcagaactggcaacggttttaccaactgacaaagcttctggactccatgcatgaggtggttgagaatctccttacctactgcttccagacatttttggataagaccatgagtattgaattcccagagatgttagctgaaatcatcactaatcagataccaaaatattcaaatggaaatatcaaaaagcttctgtttcatcaaaaatga

SEQ ID NO: 21 - DNA, 2226 bp, Artificial Sequence
  LhGR
atggctagtgaagctcgaaaaacaaagaaaaaaatcaaagggattcagcaagccactgcaggagtctcacaagacacttcggaaaatcctaacaaaacaatagttcctgcagcattaccacagctcacccctaccttggtgtcactgctggaggtgattgaacccgaggtgttgtatgcaggatatgatagctctgttccagattcagcatggagaattatgaccacactcaacatgttaggtgggcgtcaagtgattgcagcagtgaaatgggcaaaggcgataccaggcttcagaaacttacacctggatgaccaaatgaccctgctacagtactcatggatgtttctcatggcatttgccctgggttggagatcatacagacaatcaagtggaaacctgctctgctttgctcctgatctgattattaatgagcagagaatgtctctaccctgcatgtatgaccaatgtaaacacatgctgtttgtctcctctgaattacaaagattgcaggtatcctatgaagagtatctctgtatgaaaaccttactgcttctctcctcagttcctaaggaaggtctgaagagccaagagttatttgatgagattcgaatgacttatatcaaagagctaggaaaagccatcgtcaaaagggaagggaactccagtcagaactggcaacggttttaccaactgacaaagcttctggactccatgcatgaggtggttgagaatctccttacctactgcttccagacatttttggataagaccatgagtattgaattcccagagatgttagctgaaatcatcactaatcagataccaaaatattcaaatggaaatatcaaaaagcttctgtttcatcaaaaatctactagcaaaccggtaacgttatacgacgtcgctgaatacgccggcgtttctcatcaaaccgtttctagagtggttaaccaggcttcacatgttagcgctaaaacccgggaaaaagttgaagctgccatggctgagctcaactacatcccgaaccgtgttgcgcagcagctggctggtaaacaaagcttgctgatcggtgtcgcgacctcgagcttggccctgcacgcgccgtcgcaaattgtcgcggcgattaaatctcgcgccgatcaactgggtgccagcgtggtggtgtcgatggtagaacgaagcggcgtcgaagcctgtaaagcggcggtgcacaatcttctcgcgcaacgcgtcagtgggctgatcattaactatccgctggatgaccaggatgccattgctgtggaagctgcctgcactaatgttccggcgttatttcttgatgtctctgaccagacacccatcaacagtattattttctcccataaagacggtacgcgactgggcgtggagcatctggtcgcattgggtcaccagcaaatcgcgctgttagcgggcccattaagttctgtctcggcgcgtctgcgtctggctggctggcataaatatctcactcgcaatcaaattcagccgatagcggaacgggaaggcgactggagtgccatgtccggttttcaacaaaccatgcaaatgctgaatgagggcatcgttcccactgcgatgctggttgccaacgatcagatggcgctgggcgcaatgcgcgccattaccgagtccgggctgcgcgttggtgcggatatctcggtagtgggatacgacgataccgaacacagctcatgttatatcccgccgttaaccaccatcaaacaggattttcgcctgctggggcaaaccagcgtggaccgcttgctgcaactctctcagggccaggcggtgaagggcaatcagctgttgcccgtctcactggtgaaaagaaaaaccactagtggatcggaattcgccaattttaatcaaagtgggaatattgctgatagctcattgtccttcactttcactaacagtagcaacggtccgaacctcataacaactcaaacaaattctcaagcgctttcacaaccaattgcctcctctaacgttcatgataacttcatgaataatgaaatcacggctagtaaaattgatgatggtaataattcaaaaccactgtcacctggttggacggaccaaactgcgtataacgcgtttggaatcactacagggatgtttaataccactacaatggatgatgtatataactatctattcgatgatgaagataccccaccaaacccaaaaaaagagtaa

SEQ ID NO: 22 - DNA, 186 bp, Artificial Sequence
  UAS
ggagtgaaggatcgggtgacagccctccgagcgggtgacagccctccgaagcgggtgacagccctccgacgtcgggtgacagccctccgagcgggtgacagccctccgagcgggtgacagccctccgctgcagcaagacccttcctctatataaggaagttcatttcatttggagaggacagccca

SEQ ID NO: 23 - DNA, 121 bp, Artificial Sequence
  RBR miRNA precursor
ccgactcattcatccaaataccgagtcgccaaaattcaaactagactcgttaaatgaatgaatgatgcggtagacaaattggatcattgattctctttgatacagatgctataactgagga

SEQ ID NO: 24 - DNA, 21 bp, Artificial Sequence
  RBR miRNA mature
tacagatgctataactgagga

SEQ ID NO: 25 - DNA, 318 bp, Artificial Sequence
  LacOp
aaagaagaaagggaagagaaagaattgtgagcgctcacaattgaaagactagaaagaagaaagggaagagaaagaattgtgagcgctcacaattgaaagactagaaagaagaaagggaagagaaagaattgtgagcgctcacaattgaaagactagaaagaagaaagggaagagaaagaattgtgagcgctcacaattgaaagactagaaagaagaaagggaagagaaagaattgtgagcgctcacaattgaaagactagaaagaagaaagggaagagaaagaattgtgagcgctcacaattgaaagactagtggatc

SEQ ID NO: 26 - DNA, 355 bp, Artificial Sequence
  LexAop
ggagagcttgggctgcaggtcgaggctaaaaaactaatcgcattatcatcccctcgacgtactgtacatataaccactggttttatatacagcagtactgtacatataaccactggttttatatacagcagtcgacgtactgtacatataaccactggttttatatacagcagtactgtacatataaccactggttttatatacagcagtcgaggtaagattagatatggatatgtatatggatatgtatatggtggtaatgccatgtaatatgctcgactctaggatcttcgcaagacccttcctctatataaggaagttcatttcatttggagaggacacgctgaagctagtc

SEQ ID NO: 27 - DNA, 1453 bp, Artificial Sequence
  XVE
aatgaaagcgttaacggccaggcaacaagaggtgtttgatctcatccgtgatcacatcagccagacaggtatgccgccgacgcgtgcggaaatcgcgcagcgtttggggttccgttccccaaacgcggctgaagaacatctgaaggcgctggcacgcaaaggcgttattgaaattgtttccggcgcatcacgcgggattcgtctgttgcaggaagaggaagaagggttgccgctggtaggtcgtgtggctgccggtgaaccgtcgagcgcccccccgaccgatgtcagcctgggggacgagctccacttagacggcgaggacgtggcgatggcgcatgccgacgcgctagacgatttcgatctggacatgttgggggacggggattccccgggtccgggatttaccccccacgactccgccccctacggcgctctggatatggccgacttcgagtttgagcagatgtttaccgatgcccttggaattgacgagtacggtggggatccgtctgctggagacatgagagctgccaacctttggccaagcccgctcatgatcaaacgctctaagaagaacagcctggccttgtccctgacggccgaccagatggtcagtgccttgttggatgctgagccccccatactctattccgagtatgatcctaccagacccttcagtgaagcttcgatgatgggcttactgaccaacctggcagacagggagctggttcacatgatcaactgggcgaagagggtgccaggctttgtggatttgaccctccatgatcaggtccaccttctagaatgtgcctggctagagatcctgatgattggactcgtctggcgctccatggagcacccagtgaagctactgtttgctcctaacttgctcttggacaggaaccagggaaaatgtgtagagggcatggtggagatcttcgacatgctgctggctacatcatctcggttccgcatgatgaatctgcagggagaggagtttgtgtgcctcaaatctattattttgcttaattctggagtgtacacatttctgtccagcaccctgaagtctctggaagagaaggaccatatccaccgagtcctggacaagatcacagacactttgatccacctgatggccaaggcaggcctgaccctgcagcagcagcaccagcggctggcccagctcctcctcatcctctcccacatcaggcacatgagtaacaaaggcatggagcatctgtacagcatgaagtgcaagaacgtggtgcccctctatgacctgctgctggagatgctggacgcccaccgcctacatgcgcccactagccgtggaggggcatccgtggaggagacggaccaaagccacttggccactgcgggctctacttcatcgcattccttgcaaaagtattacatcacgggggaggcagagggtttccctgccacagtctga

SEQ ID NO: 28 - protein, 334 aa, Arabidopsis thaliana
MAAAMNLYTCSRSFQDSGGELMDALVPFIKSVSDSPSSSSAASASAFLHPSAFSLPPLPGYYPDSTFLTQPFSYGSDLQQTGSLIGLNNLSSSQIHQIQSQIHHPLPPTHHNNNNSFSNLLSPKPLLMKQSGVAGSCFAYGSGVPSKPTKLYRGVRQRHWGKWVAEIRLPRNRTRLWLGTFDTAEEAALAYDKAAYKLRGDFARLNFPNLRHNGSHIGGDFGEYKPLHSSVDAKLEAICKSMAETQKQDKSTKSSKKREKKVSSPDLSEKVKAEENSVSIGGSPPVTEFEESTAGSSPLSDLTFADPEEPPQWNETFSLEKYPSYEIDWDSILA

SEQ ID NO: 29 - DNA, 1005 bp, Arabidopsis thaliana
atggcagctgctatgaatttgtacacttgtagcagatcgtttcaagactctggtggtgaactcatggacgcgcttgtaccttttatcaaaagcgtttccgattctccttcttcttcttctgcagcgtctgcgtctgcgtttcttcacccctctgcgttttctctccctcctctccccggttattacccggattcaacgttcttgacccaaccgttttcatacgggtcggatcttcaacaaaccgggtcattaatcggactcaacaacctctcttcttctcagatccaccagatccagtctcagatccatcatcctcttcctccgacgcatcacaacaacaacaactctttctcgaatcttctcagcccaaagccgttactgatgaagcaatctggagtcgctggatcttgtttcgcttacggttcaggtgttccttcgaagccgacgaagctttacagaggtgtgaggcaacgtcactggggaaaatgggtggctgagatccgtttgccgagaaatcggactcgtctctggcttgggacttttgacacggcggaggaagctgcgttggcctatgataaggcggcgtacaagctgcgcggcgatttcgcccggcttaacttccctaacctacgtcataacggatctcacatcggaggcgatttcggtgaatataaacctcttcactcctcagtcgacgctaagcttgaagctatttgtaaaagcatggcggagactcagaaacaggacaaatcgacgaaatcatcgaagaaacgtgagaagaaggtttcgtcgccagatctatcggagaaagtgaaggcggaggagaattcggtttcgatcggtggatctccaccggtgacggagtttgaagagtccaccgctggatcttcgccgttgtcggacttgacgttcgctgacccggaggagccgccgcagtggaacgagacgttctcgttggagaagtatccgtcgtacgagatcgattgggattcgattctagcttag

SEQ ID NO: 30 - protein, 498 aa, Arabidopsis thaliana
MAPPMTNCLTFSLSPMEMLKSTDQSHFSSSYDDSSTPYLIDNFYAFKEEAEIEAAAASMADSTTLSTFFDHSQTQIPKLEDFLGDSFVRYSDNQTETQDSSSLTPFYDPRHRTVAEGVTGFFSDHHQPDFKTINSGPEIFDDSTTSNIGGTHLSSHVVESSTTAKLGFNGDCTTTGGVLSLGVNNTSDQPLSCNNGERGGNSNKKKTVSKKETSDDSKKKIVETLGQRTSIYRGVTRHRWTGRYEAHLWDNSCRREGQARKGRQVYLGGYDKEDRAARAYDLAALKYWGSTATTNFPVSSYSKELEEMNHMTKQEFIASLRRKSSGFSRGASIYRGVTRHHQQGRWQARIGRVAGNKDLYLGTFATEEEAAEAYDIAAIKFRGINAVTNFEMNRYDIEAVMNSSLPVGGAAAKRHKLKLALESPSSSSSDHNLQQQQLLPSSSPSDQNPNSIPCGIPFEPSVLYYHQNFFQHYPLVSDSTIQAPMNQAEFFLWPNQSY

SEQ ID NO: 31 - DNA, 2464 bp, Artificial Sequence
  AlcR
aatggcagatacgcgccgacgccagaatcatagctgcgatccctgtcgcaagggcaagcgacgctgtgatgccccggaaaatagaaacgaggccaatgaaaacggctgggtttcgtgttcaaattgcaagcgttggaacaaggattgtaccttcaattggctctcatcccaacgctccaaggcaaaaggggctgcacctagagcgagaacaaagaaagccaggaccgcaacaaccaccagtgaaccatcaacttcagctgcaacaatccctacaccggaaagtgacaatcacgatgcgcctccagtcataaactctcacgacgcgctcccgagctggactcaggggctactctcccaccccggcgaccttttcgatttcagccactctgctattcccgcaaatgcagaagatgcggccaacgtgcagtcagacgcaccttttccgtgggatctagccatccccggtgatttcagcatgggccaacagctcgagaaacctctcagtccgctcagttttcaagcagtccttcttccgccccatagcccgaacacggatgacctcattcgcgagctggaagagcagactacagatccggactcggttaccgatactaatagtgtacaacaggtcgctcaagatggatcgctatggtctgatcggcagtcgccgctactgcctgagaacagtctgtgcatggcctcagacagcacagcacggcgatatgcccgttccacaatgacgaagaatctgatgcgaatctaccacgatagtatggagaatgcactgtcctgctggctgacagagcacaattgtccatactccgaccagatcagctacctgccgcccaagcagcgggcggaatggggcccgaactggtcaaacaggatgtgcatccgggtgtgccggctagatcgcgtatctacctcattacgcgggcgcgccctgagtgcggaagaggacaaagccgcagcccgagccctgcatctggcgatcgtagcttttgcgtcgcaatggacgcagcatgcgcagaggggggctgggctaaatgttcctgcagacatagccgccgatgagaggtccatccggaggaacgcctggaatgaagcacgccatgccttgcagcacacgacagggattccatcattccgggttatatttgcgaatatcatcttttctctcacgcagagtgtgctggatgatgatgagcagcacggtatgggtgcacgtctagacaagctactcgaaaatgacggtgcgcccgtgttcctggaaaccgcgaaccgtcagctttatacattccgacataagtttgcacgaatgcaacgccgcggtaaggctttcaacaggctcccgggaggatctgtcgcatcgacattcgccggtattttcgagacaccgacgccgtcgtctgaaagcccacagcttgacccggttgtggccagtgaggagcatcgcagtacattaagccttatgttctggctagggatcatgttcgatacactaagcgctgcaatgtaccagcgaccactcgtggtgtcagatgaggatagccagatatcatcggcatctccaccaaggcgcggcgctgaaacgccgatcaacctagactgctgggagcccccgagacaggtcccgagcaatcaagaaaagagcgacgtatggggcgacctcttcctccgcacctcggactctctcccagatcacgaatcccacacacaaatctctcagccagcggctcgatggccctgcacctacgaacaggccgccgccgctctctcctctgcaacgcccgtcaaagtcctcctctaccgccgcgtcacgcagctccaaaccctcctctatcgcggcgccagccctgcccgccttgaagcggccatccagagaacgctctacgtttataatcactggacagcgaagtaccaaccatttatgcaggactgcgttgctaaccacgagctcctcccttcgcgcatccagtcttggtacgtcattctagacggtcactggcatctagccgcgatgttgctagcggacgttttggagagcatcgaccgcgattcgtactctgatatcaaccacatcgaccttgtaacaaagctaaggctcgataatgcactagcagttagtgcccttgcgcgctcttcactccgaggccaggagctggacccgggcaaagcatctccgatgtatcgccatttccatgattctctgaccgaggtggcattcctggtagaaccgtggaccgtcgttcttattcactcgtttgccaaagctgcgtatatcttgctggactgtttagatctggacggccaaggaaatgcactagcggggtacctgcagctgcggcaaaattgcaactactgcattcgggcgctgcaatttctgggcaggaagtcggatatggcggcgctggttgcgaaggatttagagagaggtttgaatgggaaagttgacagctttttg

SEQ ID NO: 32 - DNA, 250 bp, Artificial Sequence
  AlcA
ggagcgggatagttccgacctaggattggatgcatgcggaaccgcacgagggcggggcggaaattgacacaccactcctctccacgcaccgttcaagaggtacgcgtatagagccgtatagagcagagacggagcactttctggtactgtccgcacgggatgtccgcacggagagccacaaacgagcggggccccgtacgtgctctcctaccccaggatcgcatccccgcatagctgaacatctatataa

SEQ ID NO: 33 - DNA, 267 bp, Artificial Sequence
  NOSp
cggagaattaagggagtcacgttatgacccccgccgatgacgcgggacaagccgttttacgtttggaactgacagaaccgcaacgttgaaggagccactcagccgcgggtttctggagtttaatgagctaagcacatacgtcagaaaccattattgcgcgttcaaaagtcgcctaaggtcactatcagctagcaaatatttcttgtcaaaatgctccactgacgttccataaattcccctcggtatccaattagagtctcatattca

SEQ ID NO: 34 - DNA, 299 bp, Artificial Sequence
  NOSt
gcgggactctggatctagagtcaagcagatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcgacgcttgctgaattggag

SEQ ID NO: 35 - DNA, 303 bp, Arabidopsis thaliana
ccaacactcgaatccccacccgttggaccaaacccggctcattaagcgtcggttcagatttatttcctttatttaaaaaaaaggaaagggtaaaaaatagaaaattggaaacagttaaagcccaaaattgtaatttaccgagaattgtaaatttacctgaaaaccctacgctatagtttcgactataaataccaaacttaggacctcacttcagaatcccctcgtcgctgcgtctctctcccgcaaccttcgattttcgtttattcgcatccatcggagagagaaaacaatcaataagcgacc

SEQ ID NO: 36 - DNA, 632 bp, Arabidopsis thaliana
gcttatctcgtctctgttatgcttaagaagttcaatgtttcgtttcatgtaaaactttggtggtttgtgttttggggccttgtataatccctgatgaataagtgttctactatgtttccgttcctgttatctctttctttctaatgacaagtcgaacttcttctttatcatcgcttcgtttttattatctgtgcttcttttgtttaatacgcctgcaaagtgactcgactctgtttagtgcagttctgcgaaacttgtaaatagtccaattgttggcctctagtaatagatgtagcgaaagtgttgagctgttgggttctaaggatggcttgaacatgttaatcttttaggttctgagtatgatgaacattcgttgttgctaagaaatgcctgtaatgtcccacaaatgtagaaaatggttcgtacctttgtccaagcattgatatgtctgatgagaggaaactgcaagatactgagcttggtttaacgaaggagaggcagtttcttccttccaaagcatttcatttgacaatgccttgatcatcttaagtagagtttctgttgtggaaagtttgaaactttgaagaaacgactctcaagtaaattgatgatcacaagtgaaagtgtatgtt

SEQ ID NO: 37 - DNA, 247 bp, Artificial Sequence
  G1090
ggagatagtttaaactgaaggcgggaaacgacaatctgatccaagctcaagctaagcttgcatgcctgcaggatatcgtggatccaagcttgccacgtgccgccacgtgccgccacgtgccgccacgtgcctctagaggatccatctccactgacgtaagggatgacgcacaatcccactatccttcgcaagacccttcctctatataaggaagttcatttcatttggagaggacacgctgggatcc

SEQ ID NO: 38 - DNA, 278 bp, Pisum sativum
atatgtcaacagtgagaaactgttcgcattttccgttttgcttctttctttctattcaatgtatgttgttggattccagttgaatttattatgagaactaataataatagtaataatcatttgtttctttactaatttgcattttcacatatgatttctggtgcatatcataattttcattccaccaatattaatttccccattcaagttacttatgaaatagaaatcctcttctccgactactttatttgtccgaaagtcttgtggctgctatataa

SEQ ID NO: 39 - DNA, 271 bp, Escherichia coli
agcttgggctgcaggtcgaggctaaaaaactaatcgcattatcatcccctcgacgtactgtacatataaccactggttttatatacagcagtactgtacatataaccactggttttatatacagcagtcgacgtactgtacatataaccactggttttatatacagcagtactgtacatataaccactggttttatatacagcagtcgaggtaagattagatatggatatgtatatggatatgtatatggtggtaatgccatgtaatatgc

SEQ ID NO: 40 - DNA, 45 bp, Artificial Sequence
  35Smini
gacccttcctctatataaggaagttcatttcatttggagaggaca

SEQ ID NO: 41 - DNA, 478 bp, Artificial Sequence
  SIATPase
accgcactgtgtgtggtttctcaagaccaagacagctaaagcctaaagtcagagatctaatatgtgtattgttattcatgacaccacagctgccacttttggtgttatgatctgtttgtagaagtaggaattcttttttttctacttaataatagcttaaagagctgtgcaatttggtctgtattttttgtgtattttgcactcattatttgtgaacagtttgagaactatttattttctaagatttgtgcacgtatgaaccacttttcatctatataccaccatgtttattctgcatctatgggattgagtttgaatattcgttgatcaacaaagttatatttggtggatactacttgaaggtgcatatactttgtgctcatatatttagttgatattctggattttgagctggacaaattgatcaaggtagtctaatctggtctggttactaataaaactcaagagatcactgtct

SEQ ID NO: 42 - DNA, 126 bp, Artificial Sequence
  4xMyc
tcggaacaaaagttgatctctgaagaggaccttgagcagaaattgatctctgaggaagatcttgagcagaagcttatctcagaggaggatttggagcaaaaacttatttctgaagaggacctttga

SEQ ID NO: 43 - protein, 41 aa, Artificial Sequence
  4xMyc
SEQKLISEEDLEQKLISEEDLEQKLISEEDLEQKLISEEDL

SEQ ID NO: 44 - DNA, 504 bp, Arabidopsis thaliana
gcttaagctttttgtgatctgatgataagtggttggttcgtgtctcatgcacttgggaggtgatctatttcacctggtgtagtttgtgtttccgtcagttggaaaaacttatccctatcgatttcgttttcattttctgcttttcttttatgtaccttcgtttgggcttgtaacgggcctttgtatttcaactctcaataataatccaagtgcatgttaaacaatttgtcttggtgtttcggctttaatatactactggtgaagatgggccgtactactgcatcacaacgaaaaataataataagatgaaaaacttgaagtggaaaaaaaaaaaaacttgaatgttcactactactcattgaccataatgtttaacatacatagctcaatagtatttttgtgaatatggcaacacaaacagtccaaaacaattgtctcttactataccaaaccaagggcgccgcttgtttgccactctttgtgtgcaatagtgtgattaccaca

SEQ ID NO: 45 - DNA, 87 bp, Artificial Sequence
  3xFLAG
tcggattataaggaccatgacggagactataaggaccatgacctcgacgctgcagcagctgattataaggacgatgacgataagtga

SEQ ID NO: 46 - protein, 28 aa, Artificial Sequence
  3xFLAG
SDYKDHDGDYKDHDLDAAAADYKDDDDK

SEQ ID NO: 47 - DNA, 122 bp, Artificial Sequence
  UASp
atcgggtgacagccctccgagcgggtgacagccctccgaagcgggtgacagccctccgacgtcgggtgacagccctccgagcgggtgacagccctccgagcgggtgacagccctccgctgca

SEQ ID NO: 48 - DNA, 714 bp, Agrobacterium tumefaciens
gtcctgctttaatgagatatgcgagaagcctatgatcgcatgatatttgctttcaattctgttgtgcacgttgtaaaaaacctgagcatgtgtagctcagatccttaccgccggtttcggttcattctaatgaatatatcacccgttactatcgtatttttatgaataatattctccgttcaatttactgattgtaccctactacttatatgtacaatattaaaatgaaaacaatatattgtgctgaataggtttatagcgacatctatgatagagcgccacaataacaaacaattgcgttttattattacaaatccaattttaaaaaaagcggcagaaccggtcaaacctaaaagactgattacataaatcttattcaaatttcaaaagtgccccaggggctagtatctacgacacaccgagcggcgaactaataacgctcactgaagggaactccggttccccgccggcgcgcatgggtgagattccttgaagttgagtattggccgtccgctctaccgaaagttacgggcaccattcaacccggtccagcacggcggccgggtaaccgacttgctgccccgagaattatgcagcatttttttggtgtatgtgggccccaaatgaagtgcaggtcaaaccttgacagtgacgacaaatcgttgggcgggtccagggcgaattttgcgacaacatgtcgaggctcagcaggac

SEQ ID NO: 49 - DNA, 93 bp, Artificial Sequence
  V5
tcgggtaagccaatccctaatcctctcttgggtttggattctactggtggaaaacctattccaaacccattgctcggactcgactcaacctaa

SEQ ID NO: 50 - protein, 30 aa, Artificial Sequence
  V5
SGKPIPNPLLGLDSTGGKPIPNPLLGLDST

SEQ ID NO: 51 - DNA, 252 bp, Agrobacterium tumefaciens
ttggactcccatgttggcaaaggcaaccaaacaaacaatgaatgatccgctcctgcatatggggcggtttgagtatttcaactgccatttgggctgaattgtagacatgctcctgtcagaaattccgtgatcttactcaatattcagtaatctcggccaatatcctaaatgtgcgtggctttatctgtctttgtattgtttcatcaattcatgtaacgtttgcttttcttatgaattttcaaataaattatc

SEQ ID NO: 52 - DNA, 75 bp, Artificial Sequence
  T7
tcgatggcatctatgacaggaggtcaacagatgggtggaatggcttcaatgactggtggacagcaaatgggataa

SEQ ID NO: 53 - protein, 24 aa, Artificial Sequence
  T7
SMASMTGGQQMGGMASMTGGQQMG

SEQ ID NO: 54 - DNA, 90 bp, Arabidopsis thaliana
ggtaagccaatccctaatcctctcttgggtttggattctactggtggaaaacctattccaaacccattgctcggactcgactcaacctaa

SEQ ID NO: 55 - DNA, 254 bp, Arabidopsis thaliana
gctttctgttgtagcggtagatcgatccttctctttctctcttatcttttaaaaactgcatttctatttgggaattttgtaagctctttaatttgagttatcatggattctatgttgaacatctttcgttctggattataaattatggaaggttttaggattttatggatattcattatgttgttcgttctatactttgatattcattggttgctacttcttgaatcatcctataacgtagttgtgagagcatt

SEQ ID NO: 56 - DNA, 254 bp, Arabidopsis thaliana
gcttagccattctctcgcagatgatgttcactttgtgttttacttcctttatgcattcacagcaataaaagaaagaaatctccatcgcttttggttttcttctctgtcttaagttagtcgttttcgtgtctaatctattacttatcattgtaatagactcttcttctattgagatttgaatataaactaaaacacattccattttactgtgttctcaacattcagaatgcaaacggactaaccgtagtgactcg

SEQ ID NO: 57 - DNA, 204 bp, Cauliflower mosaic virus
ctctagctagagtcgatcgacaagctcgagtttctccataataatgtgtgagtagttcccagataagggaattagggttcctatagggtttcgctcatgtgttgagcatataagaaacccttagtatgtatttgtatttgtaaaatacttctatcaataaaatttctaattcctaaaaccaaaatccagtactaaaatccagat

SEQ ID NO: 58 - protein, 741 aa, Artificial Sequence
  LhGR
MASEARKTKKKIKGIQQATAGVSQDTSENPNKTIVPAALPQLTPTLVSLLEVIEPEVLYAGYDSSVPDSAWRIMTTLNMLGGRQVIAAVKWAKAIPGFRNLHLDDQMTLLQYSWMFLMAFALGWRSYRQSSGNLLCFAPDLIINEQRMSLPCMYDQCKHMLFVSSELQRLQVSYEEYLCMKTLLLLSSVPKEGLKSQELFDEIRMTYIKELGKAIVKREGNSSQNWQRFYQLTKLLDSMHEVVENLLTYCFQTFLDKTMSIEFPEMLAEIITNQIPKYSNGNIKKLLFHQKSTSKPVTLYDVAEYAGVSHQTVSRVVNQASHVSAKTREKVEAAMAELNYIPNRVAQQLAGKQSLLIGVATSSLALHAPSQIVAAIKSRADQLGASVVVSMVERSGVEACKAAVHNLLAQRVSGLIINYPLDDQDAIAVEAACTNVPALFLDVSDQTPINSIIFSHKDGTRLGVEHLVALGHQQIALLAGPLSSVSARLRLAGWHKYLTRNQIQPIAEREGDWSAMSGFQQTMQMLNEGIVPTAMLVANDQMALGAMRAITESGLRVGADISVVGYDDTEHSSCYIPPLTTIKQDFRLLGQTSVDRLLQLSQGQAVKGNQLLPVSLVKRKTTSGSEFANFNQSGNIADSSLSFTFTNSSNGPNLITTQTNSQALSQPIASSNVHDNFMNNEITASKIDDGNNSKPLSPGWTDQTAYNAFGITTGMFNTTTMDDVYNYLFDDEDTPPNPKKE

SEQ ID NO: 59 - DNA, 795 bp, artificial sequence
  nptII
atggttgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgactcatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctga

SEQ ID NO: 60 - DNA, 723 bp, artificial sequence
  octopine synthase terminator
gcgggactctggggttcgctgctttaatgagatatgcgagacgcctatgatcgcatgatatttgctttcaattctgttgtgcacgttgtaaaaaacctgagcatgtgtagctcagatccttaccgccggtttcggttcattctaatgaatatatcacccgttactatcgtatttttatgaataatattctccgttcaatttactgattgtaccctactacttatatgtacaatattaaaatgaaaacaatatattgtgctgaataggtttatagcgacatctatgatagagcgccacaataacaaacaattgcgttttattattacaaatccaattttaaaaaaagcggcagaaccggtcaaacctaaaagactgattacataaatcttattcaaatttcaaaaggccccaggggctagtatctacgacacaccgagcggcgaactaataacgttcactgaagggaactccggttccccgccggcgcgcatgggtgagattccttgaagttgagtattggccgtccgctctaccgaaagttacgggcaccattcaacccggtccagcacggcggccgggtaaccgacttgctgccccgagaattatgcagcatttttttggtgtatgtgggccccaaatgaagtgcaggtcaaaccttgacagtgacgacaaatcgttgggcgggtccagggcgaattttgcgacaacatgtcgaggctcagc

SEQ ID NO: 61 - DNA, 1416 bp, artificial sequence
  CaMV 35S promoter and TMV Omega leader
gaattccaatcccacaaaaatctgagcttaacagcacagttgctcctctcagagcagaatcgggtattcaacaccctcatatcaactactacgttgtgtataacggtccacatgccggtatatacgatgactggggttgtacaaaggcggcaacaaacggcgttcccggagttgcacacaagaaatttgccactattacagaggcaagagcagcagctgacgcgtacacaacaagtcagcaaacagacaggttgaacttcatccccaaaggagaagctcaactcaagcccaagagctttgctaaggccctaacaagcccaccaaagcaaaaagcccactggctcacgctaggaaccaaaaggcccagcagtgatccagccccaaaagagatctcctttgccccggagattacaatggacgatttcctctatctttacgatctaggaaggaagttcgaaggtgaaggtgacgacactatgttcaccactgataatgagaaggttagcctcttcaatttcagaaagaatgctgacccacagatggttagagaggcctacgcagcaagtctcatcaagacgatctacccgagtaacaatctccaggagatcaaataccttcccaagaaggttaaagatgcagtcaaaagattcaggactaattgcatcaagaacacagagaaagacatatttctcaagatcagaagtactattccagtatggacgattcaaggcttgcttcataaaccaaggcaagtaatagagattggagtctctaaaaaggtagttcctactgaatctaaggccatgcatggagtctaagattcaaatcgaggatctaacagaactcgccgtcaagactggcgaacagttcatacagagtcttttacgactcaatgacaagaagaaaatcttcgtcaacatggtggagcacgacactctggtctactccaaaaatgtcaaagatacagtctcagaagatcaaagggctattgagacttttcaacaaaggataatttcgggaaacctcctcggattccattgcccagctatctgtcacttcatcgaaaggacagtagaaaaggaaggtggctcctacaaatgccatcattgcgataaaggaaaggctatcattcaagatctctctgccgacagtggtcccaaagatggacccccacccacgaggagcatcgtggaaaaagaagaggttccaaccacgtctacaaagcaagtggattgatgtgacatctccactgacgtaagggatgacgcacaatcccactatccttcgcaagacccttcctctatataaggaagttcatttcatttggagaggacacgctcgagtataagagctcatttttacaacaattaccaacaacaacaaacaacaaacaacattacaattacatttacaatt

SEQ ID NO: 62 - DNA, 204 bp, artificial sequence; identical to SEQ ID NO: 57
  CaMV terminator

SEQ ID NO: 63 - DNA, 813 bp, artificial sequence
  erGFP
atgaaagccttcacactcgctctcttcttagctctttccctctatctcctgcccaatccagccactagtggctccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccttcacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccaagctgagcaaagaccccaacgagaagcgcgatcacatggtcctgctggagttcgtgaccgccgccgggatcactcacggcatggacgagctgtacaagtcaggtcatgatgagctttag

SEQ ID NO: 64 - protein, 264 aa, artificial sequence
  nptII
MVEQDGLHAGSPAAWVERLFGYDWAQQTIGCSDAAVFRLSAQGRPVLFVKTDLSGALNELQDEAARLSWLATTGVPCAAVLDVVTEAGRDWLLLGEVPGQDLLSSHLAPAEKVSIMADAMRRLHTLDPATCPFDHQAKHRIERARTRMEAGLVDQDDLDEEHQGLAPAELFARLKARMPDGEDLVVTHGDACLPNIMVENGRFSGFIDCGRLGVADRYQDIALATRDIAEELGGEWADRFLVLYGIAAPDSQRIAFYRLLDEFF

SEQ ID NO: 65 - protein, 270 aa, artificial sequence
  erGFP
MKAFTLALFLALSLYLLPNPATSGSMVSKGEELFTGVVPILVELDGDVNGHKFSVSGEGEGDATYGKLTLKFICTTGKLPVPWPTLVTTFTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSKLSKDPNEKRDHMVLLEFVTAAGITHGMDELYKSGHDEL

(19) United States
(12) Patent Application Publication
(10) Pub. No.: US 2021/0054393 A1
(43) Pub. Date: Feb. 25, 2021

(54) GENES FOR HORMONE-FREE PLANT REGENERATION

(71) Applicant: Keygene N.V., Wageningen (NL)

(72) Inventors: Bernardus SCHERES, Wageningen (NL); Renze HEIDSTRA, Wageningen (NL); Menno Christian Cyrano PIJNENBURG, Wageningen (NL); Michiel Theodoor Jan DE BOTH, Wageningen (NL)

(21) Appl. No.: 17/081,832

(22) Filed: Oct. 27, 2020

Related U.S. Application Data
(63) Continuation of application No. PCT/EP2019/061098, filed on Apr. 30, 2019.

(30) Foreign Application Priority Data
May 1, 2018 (EP) 18170247.3

(51) Int. Cl. C12N 15/82; C07K 14/415

(52) U.S. Cl. CPC C12N 15/8227; C07K 14/415

(57) ABSTRACT

The invention pertains to a method for regenerating a plant cell, preferably regenerating a shoot from a plant cell by altering the expression levels of at least WOX5 and a PLT protein, preferably WOX5 and PLT1. In addition the expression levels of further proteins can altered, such as WIND1, SHR, SCR, RBR, PLT4 and PLT5 to regenerate a shoot from a plant cell. Preferably, the expression levels are transiently altered. The invention further pertains to a nucleic acid construct suitable for transient protein expression and the use of the protein combinations for regenerating a shoot from a plant cell.

### CROSS-REFERENCE TO RELATED APPLICATIONS

This application is a Continuation of International Patent Application No. PCT/EP2019/061098, filed Apr. 30, 2019, which claims priority to European Patent Application No. 18170247.3, filed May 1, 2018, the entirety of these applications are herein incorporated by reference in their entirety.

### SEQUENCE LISTING

The instant application contains a Sequence Listing which is being submitted electronically in ASCII format and is hereby incorporated by reference in its entirety. Said ASCII copy, created on Apr. 30, 2019, is named 085342-3900_SequenceListing.txt and is 117 KB in size.

### FIELD OF THE INVENTION

The present invention relates to the field of molecular plant biology, in particular to the field of plant regeneration. The invention concerns methods for improving the regeneration of plant cells without the need of plant growth hormones.

### BACKGROUND

The current plant regeneration technology is variable with respect to the successful engineering of desired traits in important crop species. Present regeneration protocols still mainly depend on the application of two key plant hormones, auxin and cytokinin, to control de novo plant regeneration in a two-step process (Skoog and Miller, 1957). In general, auxin rich medium is used to induce regeneration competent callus from which shoot organogenesis is induced by cytokinin rich medium. Engineering of plant species recalcitrant to the present protocols results in ineffective use of modern biotechnology for their genetic improvement. Accordingly, methods are needed to increase regeneration efficiencies to allow for genotype-independent shoot organogenesis.

Experiments in Arabidopsis thaliana have revealed that the competence of callus to produce a shoot is accompanied by the appearance of root traits that show root identity acquisition of callus cells (Sugimoto et al., 2010). Expression of root traits in regeneration competent callus is followed by the expression of shoot specific genes demonstrating the importance of transient root acquisition to generate founder cells for the initiation towards shoot regeneration (Rosspopoff et al., 2017). Genetic studies support the importance of root trait acquisition for the regeneration potential of the callus tissue (Sugimoto et al., 2010) (Kareem et al., 2015) (Fan et al., 2012). These studies reveal the involvement of additional regulators for shoot regeneration but still rely on hormonal induction during the regeneration process. This dependency on plant hormone induction therefore may still hamper the regeneration efficiency of plant cells.

Iwase et al. (2015) have shown that overexpression of WIND1 can bypass auxin pre-treatment, but still requires the presence of the plant hormone cytokinin.

Shoot regeneration in the absence of phytohormones has been shown in the art previously, but requires wounding of the plant (Iwase et al., 2017).

Therefore, there is still a need in the art for methods conferring regenerative potential to plant species, in particular recalcitrant plant species, or enhancing their regeneration efficiency, independent of externally applied plant hormones and not requiring wounding the plant. Additionally, there is a need for recombinant DNA constructs that increase or induce the regenerative potential of e.g. recalcitrant plants upon introduction in the plant cell.

### SUMMARY

In an aspect, the invention pertains to a method for regenerating a shoot from a plant cell, comprising the steps of:
a) introducing or increasing in the plant cell the expression of a combination of proteins comprising at least:
   i) a WUSCHEL related homeobox 5 (WOX5) protein; and
   ii) a PLETHORA (PLT) protein selected from the group consisting of PLT1, PLT2, PLT3, PLT4, PLT5 and PLT7, preferably PLT1;
wherein the expression of at least one of the proteins of the combination of proteins is transiently introduced or increased; and
b) allowing the plant cell to regenerate into the shoot.

In an embodiment, the combination of proteins further comprises
iii) a WOUND INDUCED DEDIFFERENTIATION 1 (WIND1) protein.

In an embodiment, the combination of proteins further comprises:
iv) a SHORT ROOT (SHR) protein;
v) a SCARECROW (SCR) protein; and
vi) at least three PLETHORA (PLT) proteins selected from the group consisting of PLT1, PLT2, PLT3, PLT4, PLT5 and PLT7,

In an embodiment, the combination of proteins comprises at least three selected PLT proteins which include at least one or more of PLT1, PLT4 and PLT5, wherein preferably the at least three selected PLT proteins are PLT1, PLT4 and PLT5.

In an embodiment, step a) further comprises decreasing the expression of an endogenous Retinoblastoma Related (RBR) protein, wherein preferably the expression of the RBR protein is transiently decreased.

Preferably, the expression of all proteins of the combination of proteins as defined herein is transiently introduced or increased, wherein optionally the expression of the RBR protein is transiently decreased.

Preferably, the expression of all proteins of the combination of proteins as defined herein is simultaneously transiently introduced or increased and optionally the expression of the RBR protein is transiently decreased simultaneously with the combination of proteins.

In an embodiment, the expression of at least one of the proteins of the combination of proteins as defined herein, preferably the expression of all proteins of the combination of proteins, is transiently introduced or increased by transient activation of their expression and optionally the expression of the RBR protein is transiently decreased by transient activation of the expression of a RBR repressor.

In a further embodiment,
i) the amino acid sequence of the SHR protein has at least 60% sequence identity with SEQ ID NO: 1;
ii) the amino acid sequence of the SCR protein has at least 60% sequence identity with SEQ ID NO: 2;

iii) the amino acid sequence of the WOX5 protein has at least 60% sequence identity with SEQ ID NO: 3;

iv) the amino acid sequence of the PLT1, PLT2, PLT3, PLT4, PLT5 and PLT7 proteins have at least 60% sequence identity with SEQ ID NO: 4, SEQ ID NO: 5, SEQ ID NO: 6, SEQ ID NO: 7 SEQ ID NO: 8 and SEQ ID NO: 30, respectively;
v) the amino acid sequence of the RBR protein has at least 60% sequence identity with SEQ ID NO: 17; and
vi) the amino acid sequence of the WIND1 protein has at least 60% sequence identity with SEQ ID NO: 28.

In an embodiment,
i) the SHR protein is encoded by a nucleotide sequence having at least 60% sequence identity with SEQ ID NO: 9;
ii) the SCR protein is encoded by a nucleotide sequence having at least 60% sequence identity with SEQ ID NO: 10;
iii) the WOX5 protein is encoded by a nucleotide sequence having at least 60% sequence identity with SEQ ID NO: 11;
iv) the PLT1, PLT2, PLT3, PLT4, PLT5 and PLT7 proteins are encoded by a nucleotide sequence having at least 60% sequence identity with SEQ ID NO: 12, SEQ ID NO: 13, SEQ ID NO: 14, SEQ ID NO: 15, SEQ ID NO: 16 and SEQ ID NO: 19, respectively;
v) the RBR protein is encoded by a nucleotide sequence having at least 60% sequence identity with SEQ ID NO: 18; and
vi) the WIND1 protein is encoded by a nucleotide sequence having at least 60% sequence identity with SEQ ID NO: 29.

In a further embodiment, the plant cell is part of a multicellular tissue, preferably a callus tissue, a plant organ or an explant. Preferably, the plant organ is a root.

In an embodiment, the plant cell is obtainable from a plant selected from the group consisting of Arabidopsis, barley, cabbage, canola, cassava, cauliflower, chicory, chrysanthemum, cotton, cucumber, eggplant, grape, hot pepper, lettuce, maize, melon, oilseed rape, potato, pumpkin, rice, rye, sorghum, soybean, squash, sugar cane, sugar beet, sunflower, sweet pepper, tomato, water melon, wheat, and zucchini. Optionally, the plant cell is obtainable from a plant of the family of Solanaceae, optionally of the genus Solanum, optionally the species Solanum lycopersicum or Solanum melongena. Optionally, the plant cell is obtainable from the family of Brassicaceae, optionally the species, or subspecies, is Raphanus sativus, Brassica oleracea, Brassica rapa, Brassica napus, Armoracia rusticana or Arabidopsis thaliana.

In an embodiment, the method comprises a step c) of forming a plant or plant part from the regenerated shoot.

In a further aspect, the invention pertains to a composition comprising at least two nucleic acid molecules, wherein
i) a first nucleic acid molecule comprises:
a nucleotide sequence encoding a WOX5 protein operably linked to an inducible promoter; and
ii) a second nucleic acid molecule comprises a nucleotide sequence encoding a PLT protein selected from the group consisting of PLT1, PLT2, PLT3, PLT4, PLT5 and PLT7, preferably PLT1, operably linked to an inducible promoter.

In another aspect, the invention concerns a nucleic acid construct comprising the nucleotide sequence of the first and the second nucleic acid molecules as defined herein.

Preferably, the nucleic acid construct comprises a further nucleotide sequence encoding a transactivator that is preferably operably linked to a promoter, wherein the transactivator upon binding an inducer, activates the inducible promoter. Preferably said inducible promoter is part of the at least one expression cassette. Preferably said inducible promoter is operably linked to at least one of the nucleotide sequences encoding SHR, SCR, WOX5, WIND, PLT1, PLT2, PLT3, PLT4, PLT5 and PLT7 protein, and RBR repressor as defined herein.

Preferably, the transactivator is encoded by a nucleotide sequence having:
i) at least 60% sequence identity with SEQ ID NO: 21 and wherein the transactivator is capable of binding to dexamethasone corticoid or a derivative thereof; or
ii) at least 60% sequence identity with SEQ ID NO: 27 and wherein the transactivator is capable of binding to β-estradiol or a derivative thereof.

In a further aspect, the invention relates to a plant cell comprising at least one of:
i) a first nucleic acid molecule comprising a nucleotide sequence encoding a WOX5 protein operably linked to an inducible promoter and a second nucleic acid molecule comprising a nucleotide sequence encoding a PLT protein selected from the group consisting of PLT1, PLT2, PLT3, PLT4, PLT5 and PLT7, preferably PLT1, operably linked to an inducible promoter; and and
ii) the nucleic acid construct as defined herein.

In another aspect, the invention concerns a shoot, plant or plant part obtainable by the method as defined herein.

In an aspect, the invention pertains to the use of a combination of proteins as defined herein, and optionally a RBR repressor as defined herein, or a nucleic acid composition or construct as defined herein, for regenerating shoot from a plant cell.

### Definitions

Various terms relating to the methods, compositions, uses and other aspects of the present invention are used throughout the specification and claims. Such terms are to be given their ordinary meaning in the art to which the invention pertains, unless otherwise indicated. Other specifically defined terms are to be construed in a manner consistent with the definition provided herein.

It is clear for the skilled person that any methods and materials similar or equivalent to those described herein can be used for practicing the present invention.

Methods of carrying out the conventional techniques used in methods of the invention will be evident to the skilled worker. The practice of conventional techniques in molecular biology, biochemistry, computational chemistry, cell culture, recombinant DNA, bioinformatics, genomics, sequencing and related fields are well-known to those of skill in the art and are discussed, for example, in the following literature references: Sambrook et al. Molecular Cloning. A Laboratory Manual, 2nd Edition, Cold Spring Harbor Laboratory Press, Cold Spring Harbor, N.Y., 1989; Ausubel et al. Current Protocols in Molecular Biology, John Wiley & Sons, New York, 1987 and periodic updates; and the series Methods in Enzymology, Academic Press, San Diego.

The singular terms “a,” “an,” and “the” include plural referents unless the context clearly dictates otherwise. Thus, for example, reference to “a plant cell” includes a combination of two or more plant cells, and the like. The indefinite article “a” or “an” thus usually means “at least one”.

The term “and/or” refers to a situation wherein one or more of the stated cases may occur, alone or in combination with at least one of the stated cases, up to with all of the stated cases.

As used herein, the term “about” is used to describe and account for small variations. For example, the term can refer to less than or equal to ±(+ or −) 10%, such as less than or equal to ±5%, less than or equal to ±4%, less than or equal to ±3%, less than or equal to ±2%, less than or equal to ±1%, less than or equal to ±0.5%, less than or equal to ±0.1%, or less than or equal to ±0.05%.

Additionally, amounts, ratios, and other numerical values are sometimes presented herein in a range format. It is to be understood that such range format is used for convenience and brevity and should be understood flexibly to include numerical values explicitly specified as limits of a range, but also to include all individual numerical values or sub-ranges encompassed within that range as if each numerical value and sub-range is explicitly specified. For example, a ratio in the range of about 1 to about 200 should be understood to include the explicitly recited limits of about 1 and about 200, but also to include individual ratios such as about 2, about 3, and about 4, and sub-ranges such as about 10 to about 50, about 20 to about 100, and so forth.

The term “comprising” is construed as being inclusive and open ended, and not exclusive. Specifically, the term and variations thereof mean the specified features, steps or components are included. These terms are not to be interpreted to exclude the presence of other features, steps or components.

The term “plant hormone”, “plant growth hormone”, “plant growth regulator” or “phytohormone” is to be understood herein as a chemical that influence the growth and development of plant cells and tissues. Plant growth regulators comprise chemicals from the following five groups: auxins, cytokinins, gibberellins, abscisic acid (ABA) and ethylene. In addition to the five main groups, two other classes of chemical are often regarded as plant growth regulators: brassinosteroids and polyamines. For the induction of shoot regeneration in plant tissues, a combination of one or more cytokinins and one or more auxins is usually employed.

The terms “protein” or “polypeptide” are used interchangeably and refer to molecules consisting of a chain of amino acids, without reference to a specific mode of action, size, 3 dimensional structure or origin. A “fragment” or “portion” of a protein may thus still be referred to as a “protein.” An “isolated protein” is used to refer to a protein which is no longer in its natural environment, for example in vitro or in a recombinant bacterial or plant host cell.

“Plant” refers to either the whole plant or to parts of a plant, such as cells, tissue or organs (e.g. pollen, seeds, gametes, roots, leaves, flowers, flower buds, anthers, fruit, etc.) obtainable from the plant, as well as derivatives of any of these and progeny derived from such a plant by selfing or crossing.

“Plant cell(s)” include protoplasts, gametes, suspension cultures, microspores, pollen grains, etc., either in isolation or within a tissue, organ or organism. The plant cell can e.g. be part of a multicellular structure, such as a callus, meristem plant organ or an explant.

“Similar conditions” for culturing the plant/plant cell means among other things the use of a similar temperature, humidity, nutrition and light conditions, and similar irrigation and day/night rhythm.

The terms “homology”, “sequence identity” and the like are used interchangeably herein. Sequence identity is herein defined as a relationship between two or more amino acid (polypeptide or protein) sequences or two or more nucleic acid (polynucleotide) sequences, as determined by comparing the sequences. In the art, “identity” also means the degree of sequence relatedness between amino acid or nucleic acid sequences, as the case may be, as determined by the match between strings of such sequences. “Similarity” between two amino acid sequences is determined by comparing the amino acid sequence and its conserved amino acid substitutes of one polypeptide to the sequence of a second polypeptide. “Identity” and “similarity” can be readily calculated by known methods. The percentage sequence identity/similarity can be determined over the full length of the sequence.

“Sequence identity” and “sequence similarity” can be determined by alignment of two peptide or two nucleotide sequences using global or local alignment algorithms, depending on the length of the two sequences. Sequences of similar lengths are preferably aligned using a global alignment algorithms (e.g. Needleman Wunsch) which aligns the sequences optimally over the entire length, while sequences of substantially different lengths are preferably aligned using a local alignment algorithm (e.g. Smith Waterman). Sequences may then be referred to as “substantially identical” or “essentially similar” when they (when optimally aligned by for example the programs GAP or BESTFIT using default parameters) share at least a certain minimal percentage of sequence identity (as defined below). GAP uses the Needleman and Wunsch global alignment algorithm to align two sequences over their entire length (full length), maximizing the number of matches and minimizing the number of gaps. A global alignment is suitably used to determine sequence identity when the two sequences have similar lengths. Generally, the GAP default parameters are used, with a gap creation penalty=50 (nucleotides)/8 (proteins) and gap extension penalty=3 (nucleotides)/2 (proteins). For nucleotides the default scoring matrix used is nwsgapdna and for proteins the default scoring matrix is Blosum62 (Henikoff & Henikoff, 1992, PNAS 89, 915-919). Sequence alignments and scores for percentage sequence identity may be determined using computer programs, such as the GCG Wisconsin Package, Version 10.3, available from Accelrys Inc., 9685 Scranton Road, San Diego, Calif. 92121-3752 USA, or using open source software, such as the program “needle” (using the global Needleman Wunsch algorithm) or “water” (using the local Smith Waterman algorithm) in EmbossWIN version 2.10.0, using the same parameters as for GAP above, or using the default settings (both for ‘needle’ and for ‘water’ and both for protein and for DNA alignments, the default Gap opening penalty is 10.0 and the default gap extension penalty is 0.5; default scoring matrices are Blossum62 for proteins and DNAFull for DNA). When sequences have a substantially different overall lengths, local alignments, such as those using the Smith Waterman algorithm, are preferred.

Alternatively percentage similarity or identity may be determined by searching against public databases, using algorithms such as FASTA, BLAST, etc. Thus, the nucleic acid and protein sequences of the present invention can further be used as a “query sequence” to perform a search against public databases to, for example, identify other family members or related sequences. Such searches can be performed using the BLASTn and BLASTx programs (version 2.0) of Altschul, et al. (1990) J. Mol. Biol. 215:403-10. BLAST nucleotide searches can be performed with the NBLAST program, score=100, wordlength=12 to obtain nucleotide sequences homologous to nucleic acid molecules of the invention. BLAST protein searches can be performed with the BLASTx program, score=50, wordlength=3 to obtain amino acid sequences homologous to protein molecules of the invention. To obtain gapped alignments for comparison purposes, Gapped BLAST can be utilized as described in Altschul et al., (1997) Nucleic Acids Res. 25(17): 3389-3402. When utilizing BLAST and Gapped BLAST programs, the default parameters of the respective programs (e.g., BLASTx and BLASTn) can be used. See the homepage of the National Center for Biotechnology Information at http://www.ncbi.nlm.nih.gov/.

The term “complementarity” is herein defined as the sequence identity of a sequence to a fully complementary strand. For example, a sequence that is 100% complementary (or fully complementary) is herein understood as having 100% sequence identity with the complementary strand and e.g. a sequence that is 80% complementary is herein understood as having 80% sequence identity to the (fully) complementary strand.

The terms “nucleic acid construct”, “nucleic acid vector”, “vector” and “expression vector” are used interchangeably herein and is herein defined as a man-made nucleic acid molecule resulting from the use of recombinant DNA technology. The terms “nucleic acid construct” and “nucleic acid vector” therefore does not include naturally occurring nucleic acid molecules although a nucleic acid construct may comprise (parts of) naturally occurring nucleic acid molecules.

The vector backbone may for example be a binary or superbinary vector (see e.g. U.S. Pat. No. 5,591,616, US 2002138879 and WO 95/06722), a co-integrate vector or a T-DNA vector, as known in the art and as described elsewhere herein, into which a chimeric gene is integrated or, if a suitable transcription regulatory sequence is already present, only a desired nucleic acid sequence (e.g. a coding sequence, an antisense or an inverted repeat sequence) is integrated downstream of the transcription regulatory sequence. Vectors can comprise further genetic elements to facilitate their use in molecular cloning, such as e.g. selectable markers, multiple cloning sites and the like.

The term “gene” means a DNA fragment comprising a region (transcribed region), which is transcribed into an RNA molecule (e.g. an mRNA) in a cell. The gene can be operably linked to suitable regulatory regions (e.g. a promoter). A gene will usually comprise several operably linked fragments, such as a promoter, a 5′ leader sequence, a coding region and a 3′ non-translated sequence (3′ end) comprising a polyadenylation site.

“Expression of a gene” refers to the process wherein a DNA region which is operably linked to appropriate regulatory regions, particularly a promoter, is transcribed into an RNA, and, in case the RNA encodes for a biologically active protein or peptide, subsequently translated into a biologically active protein or peptide.

The term “operably linked” refers to a linkage of polynucleotide elements in a functional relationship. A nucleic acid is “operably linked” when it is placed into a functional relationship with another nucleic acid sequence. For instance, a promoter, or rather a transcription regulatory sequence, is operably linked to a coding sequence if it affects the transcription of the coding sequence. Operably linked may mean that the DNA sequences being linked are contiguous.

“Promoter” refers to a nucleic acid fragment that functions to control the transcription of one or more nucleic acids. A promoter fragment is located upstream (5′) with respect to the direction of transcription of the transcription initiation site of the gene, and is structurally identified by the presence of a binding site for DNA-dependent RNA polymerase, transcription initiation site(s) and can further comprise any other DNA sequences, including, but not limited to transcription factor binding sites, repressor and activator protein binding sites, and any other sequences of nucleotides known to one of skill in the art to act directly or indirectly to regulate the amount of transcription from the promoter.

Optionally the term “promoter” may also include the 5′ UTR region (5′ Untranslated Region) (e.g. the promoter may herein include one or more parts upstream of the translation initiation codon of transcribed region, as this region may have a role in regulating transcription and/or translation). A “constitutive” promoter is a promoter that is active in most tissues under most physiological and developmental conditions. An “inducible” promoter is a promoter that is physiologically (e.g. by external application of certain compounds) or developmentally regulated. A “tissue specific” promoter is only active in specific types of tissues or cells.

The term “regeneration” is herein defined as the formation of a new tissue and/or a new organ from a single plant cell, a callus, an explant, a tissue or an organ. Preferably, the regeneration is at least one of shoot regeneration, ectopic apical meristem formation, and root regeneration. Regeneration can occur through somatic embryogenesis or organogenesis. In context of the current invention, regeneration includes at least organogenesis, preferably the regeneration is through the process of organogenesis. Preferably, the regeneration as defined herein concerns at least de novo shoot formation. The regeneration may further include the formation of a new plant from a single plant cell or from e.g. a callus, an explant, a tissue or an organ. The plant cell for regeneration can be an undifferentiated plant cell. The regeneration process hence can occur directly from parental tissues or indirectly, e.g. via the formation of a callus.

“Conditions that allow for regeneration” is herein understood as an environment wherein a plant cell or a tissue can regenerate. Such conditions include at minimum a suitable temperature, nutrition, day/night rhythm and irrigation.

“Altered expression level” of a protein is herein understood as an expression level that deviates from the endogenous expression levels of the protein in an unmodified, wild-type, plant cell. The unmodified plant cell and the plant cell having an altered protein expression preferably have the same genetic background. The altered expression levels can be increased or decreased in comparison to the endogenous expression levels. In context of the invention, altered expression levels of WOX5, WIND1, SHR, SCR, PLT1, PLT2, PLT3, PLT4, PLT5 or PLT7 is preferably an increased or introduced expression. An altered expression level of RBR is preferably a decreased expression. Expression levels can be measured by any method suitable in the art, such as, but not limited to qPCR, Northern blot, microarray, etc.

### DETAILED DESCRIPTION

Regeneration of plant cells requires exposure of the cells to plant hormones such as cytokinin and/or auxin. The inventors now discovered that the timely expression of a specific group of proteins renders the presence of such plant hormones obsolete. Put differently, the inventors noticed that expression of this specific group of proteins induces spontaneous regeneration, in particular spontaneous organogenesis.

In a first aspect, the invention therefore pertains to a method for regenerating a plant cell. In an embodiment, the method relates to regenerating a meristem from a plant cell, wherein preferably the meristem grows out to form shoots. Hence, the invention concerns a method for regenerating a shoot from a plant cell. The shoot can be dissected from the underlying cell mass and induced to form roots. Alternatively, the shoots can be dissected from the underlying cell mass and roots can be formed without any further induction.

The invention thus also pertains to a method for regenerating a shoot from a plant cell, without the requirement that the plant cell is exposed to a plant growth hormone to induce or stimulate regeneration. Hence the method as defined herein is a hormone-independent method for regenerating a shoot from a plant cell.

Preferably the shoot regeneration is through the process of organogenesis. Hence the method for regeneration as defined herein is preferably a method for organogenesis, preferably shoot organogenesis. The shoot organogenesis can be a direct shoot organogenesis or an indirect shoot organogenesis.

In this respect in plant tissue culture, two alternative pathways exist for the de novo formation of new plants (i.e. regeneration) from callus or tissue explants: organogenesis and somatic embryogenesis. Organogenesis involves inducing the callus or tissue to form organs (shoots or roots). A preferred type of organogenesis in tissue culture is shoot organogenesis. Somatic embryogenesis is the process by which callus or tissue explants, usually through an embryogenic callus phase, develops structures resembling zygotic embryos, which germinate into complete plantlets (Chieng, L M N et al (2014) Induction of organogenesis and somatic embryogenesis of Gonystylus bancanus (Miq.) Kurz (Ramin) in Sarawak. SARAWAK FORESTRY Corporation & ITTO, Kuching, Malaysia & ITTO; ISBN 978-967-12855-3-4). These pathways differ in a number of characteristics as set out here.

Explants are pieces of primary tissue taken from a plant that may serve as the source of regeneration through either organogenesis or somatic embryogenesis. Explants can include stem and root segments, leaf sections, inflorescence sections, seedling parts such as cotyledons, hypocotyls, and immature and mature seed embryos (Thorpe, T A (1993) In vitro Organogenesis and Somatic Embryogenesis: Physiological and Biochemical Aspects. In: Roubelakis-Angelakis K. A., Van Thanh K. T. (eds) Morphogenesis in Plants. NATO ASI Series (Series A: Life Sciences), Vol. 253. Springer, Boston, Mass.). Through the manipulation of plant growth regulators and culture conditions, the explant may develop shoots (or roots) directly. This is called direct organogenesis. If the formation of shoots passes through a callus phase, this is called indirect organogenesis. Similarly, in somatic embryogenesis, embryos can develop directly from the explant (direct somatic embryogenesis) or via a callus phase (indirect somatic embryogenesis, Thorpe, supra; Chieng et al., supra).

Shoot organogenesis: Shoot organogenesis is the regeneration pathway by which cells of callus or explant form a de novo shoot apical meristem that develops into a shoot with leaf primordia and leaves. As there is only one apical meristem, this is a unipolar structure, and roots are not formed at this stage. The vascular system of the shoot is often connected to the parent tissue. Only after the shoots have fully formed and elongated, and are taken off the callus or explant, can the formation of roots be induced in a separate root induction step on a different culture medium (Thorpe, supra). In the art, shoot organogenesis is induced by plant growth regulators (PGRs), usually cytokinins alone in different concentrations or in combination with an auxin, wherein the cytokinins remain a constituent of the culture media until the new shoot apical meristems and the shoots have been formed and are sufficiently elongated to take them off the primary explant or callus.

Cytokinins are for example 6-aminopurine (BAP), zeatin, kinetin, thidiazuron (TDZ) and 6-(γ,γ-dimethylallylamino)purine (2-iP). For shoot organogenesis by a combination of cytokinin and auxin, the cytokinin to auxin ratio must be >1 (Dodds, J H and Roberts, L W (1985) Experiments in plant tissue culture. Cambridge University Press, Cambridge, UK).

Somatic embryogenesis: In contrast, somatic embryogenesis leads to the formation of bipolar structures resembling zygotic embryos, which contain a root-shoot axis with a closed independent vascular system. In other words, both root and shoot primordia are being formed simultaneously, and there is no vascular connection to the underlying tissue (Dodds and Roberts, supra). Somatic embryogenesis can be induced indirectly from callus or cell suspensions, or they can be induced directly on cells of explants (Thorpe, supra). Somatic embryo formation passes through a number of distinct stages, from globular stage (small isodiametric cell clusters), via heart stage (bilaterally symmetrical structures) to torpedo stage (elongation). The globular-to-heart transition is marked by the outgrowth of the two cotyledons and the beginning of the development of the radicle (Zimmerman, J L (1993) Somatic Embryogenesis: A Model for Early Development in Higher Plants. The Plant Cell 5: 1411-1423; Von Arnold et al (2002) Developmental pathways of somatic embryogenesis. Plant Cell, Tissue and Organ Culture 69: 233-249). Finally, torpedo-stage somatic embryos can develop into plantlets that contain green cotyledons, elongated hypocotyls, and developed radicles with clearly differentiated root hairs (Zimmerman, supra), in a process that is termed ‘germination’ (analogous to zygotic embryos) or ‘conversion’ or ‘maturation’ (Von Arnold et al., supra). In the induction of somatic embryogenesis, directly or indirectly, auxins are used at the initial stage to induce an embryogenic state in the callus, but the embryos only form after passage of the culture to a medium without or with reduced auxin levels. Auxins used for somatic embryo induction are e.g. 1-naphthaleneacetic acid (NAA), 2,4-dichlorophenoxyacetic acid (2,4-D), picloram and dicamba.

TABLE 1
Differences between organogenesis and somatic
embryogenesis according to the art

| Organogenesis | Somatic embryogenesis |
|---|---|
| unipolar | bipolar |
| vascular tissue | vascular tissue unconnected |
| connected to underlying tissue | |
| induction by high | induction by high auxin |
| cytokinin to auxin ratio | level or high auxin to |
| continuous induction | somatic embryos form |
| on plant growth | after removal or |
| no distinct phases | passes through distinct |
| | phases of embryo |
| no cotyledons or | embryos contain |
| radicle or root formed | cotyledons and radicle |

In an embodiment, the invention concerns a method for regenerating a plant cell, preferably a shoot from a plant cell, wherein the method comprises a step of introducing or increasing the expression of at least a SHORT ROOT (SHR) protein. The terms SHR, SGR7, SHOOT GRAVITROPISM 7 and SHORT ROOT are used interchangeably herein. Preferably, the expression is transiently introduced or increased. The SHR protein is a transcription factor, which can be expressed in the stele and moves to the surrounding cells, including the quiescent centre.

The quiescent centre is a group of cells that act as an organizer to prevent the differentiation of surrounding stem cells. Each stem cell that is adjacent to the quiescent centre divides asymmetrically to renew itself and to produce a daughter cell that divides a number of times in the meristematic zone before exiting the cell cycle in the transition zone. Subsequently, cells elongate and acquire a specific differentiation status. Stem cells that are distal to the quiescent centre produce daughter cells that differentiate (Heidstra and Sabatini, 2014). In the quiescent centre, SHR can activate SCARECROW (SCR).

In addition or alternatively, the method comprises a step of introducing or increasing the expression of at least a SCARECROW (SCR) protein. The terms SCR, SCARECROW, SGR1 and SHOOT GRAVITROPISM 1 are used interchangeably herein. Preferably, the expression is transiently introduced or increased. The SCR protein is known to be required for quiescent centre specification, is involved in both stem cell maintenance and the differentiation of their progeny. In the quiescent centre, SCR can directly repress the expression of the differentiation promoting cytokinin-response transcription factor ARR1.

In addition or alternatively, the method comprises a step of introducing or increasing the expression of at least a WUSCHEL related homeobox 5 (WOX5) protein. Preferably, the expression is transiently introduced or increased. WOX5 is a homologue of WUS, and marks the quiescent centre from inception onwards. Loss of WOX5 function is known to result in differentiation of the distal columella stem cells without altering root growth and meristem size. Nevertheless, mutant analyses indicated that WOX5 may redundantly control proximal stem cell maintenance (Sarkar et al. 2007).

In addition or alternatively, the method comprises a step of introducing or increasing the expression of at least a PLETHORA (PLT) protein. Preferably, the expression is transiently introduced or increased. PLT proteins accumulate in the quiescent centre to form an instructive gradient and the highest protein levels in the stem cell niche coincide with the auxin maximum. PLTs can regulate PIN gene family expression, which suggests a feedforward loop to maintain high auxin and PLT levels in the stem cell niche (Heidstra and Sabatini, 2014). The PLT protein can be selected from the group consisting of PLT1, PLT2, PLT3, PLT4, PLT5 and PLT7.

In addition or alternatively, the method comprises a step of introducing or increasing the expression of at least 2, 3, 4 or 5 PLT proteins, preferably at least 3 PLT proteins and preferably a step of introducing or increasing the expression of 3 PLT proteins. Preferably, the expression of the at least 2, 3, 4, 5 or 6 PLT proteins is transiently introduced or increased. The PLT proteins can be selected from the group consisting of PLT1, PLT2, PLT3, PLT4, PLT5 and PLT7. Preferably, the PLT proteins for use in the method of the invention are PLT1, PLT4 and PLT5. Preferably, the expression of these three proteins is transiently introduced or increased. The term PLT4 and BBM are used interchangeably herein. Similarly, the terms PLT5, AIL5, AINTEGUMENTA-LIKE 5, CHO1, CHOTTO 1, EMBRYOMAKER and EMK can be used interchangeably herein. In addition, the terms PLT7, AIL7, AINTEGUMENTA-LIKE 7 and PLETHORA 7 can be used interchangeably herein.

In addition or alternatively, the method comprises a step of decreasing in the plant cell the expression of a Retinoblastoma Related (RBR) protein. The terms RBR, ATRBR1, RB, RB1, RBR1, RETINOBLASTOMA 1, RETINOBLASTOMA-RELATED, RETINOBLASTOMA-RELATED 1 and RETINOBLASTOMA-RELATED PROTEIN 1 are used interchangeably herein. Preferably the expression of the RBR protein is transiently decreased. The RBR protein, the plant homologue of the RB tumour suppressor protein, has a crucial role in both shoot and root stem cell niches. As in animals, RBR inhibits cell cycle progression by interacting with an E2F transcription factor homologue. Moreover, reduced levels of RBR result in an increase in stem cell numbers, and increased RBR levels lead to stem cell differentiation, which indicates a prominent role for RBR in stem cell maintenance (Heidstra and Sabatini, 2014).

In addition or alternatively, the method comprises a step of introducing or increasing the expression of at least a WOUND INDUCED DEDIFFERENTIATION 1 (WIND1). The terms WIND1, ATWIND1, RAP2.4, RELATED TO AP2 4 and WOUND INDUCED DEDIFFERENTIATION 1 can be used interchangeably herein. Preferably the expression of the WIND1 protein is transiently introduced or increased. The WIND1 protein is a central regulator of wound-induced cellular reprogramming in plants. It has been demonstrated previously that WIND1 promotes callus formation and shoot regeneration by upregulating the expression of the ESR1 gene in Arabidopsis thaliana (Iwase et al, 2017)

In one embodiment, the invention pertains to a method for regenerating a plant cell, preferably a shoot from a plant cell, wherein the method comprises the step of increasing in the plant cell the expression of at least a combination of the proteins detailed herein above, such as one of the following combinations of proteins:
a SHR protein and a SCR protein;
a SHR protein and a WOX5 protein;
a SHR protein and at least one or more PLT proteins;
a SHR protein and a WIND1 protein;
a SCR protein and a WOX5 protein;
a SCR protein and at least one or more PLT proteins;
a SCR protein and a WIND1 protein;
a WOX5 protein and at least one or more PLT proteins;
a WOX5 protein and a WIND1 protein;
one or more PLT proteins and a WIND1 protein;
a SHR protein, a SCR protein and a WOX5 protein;
a SHR protein, a SCR protein and at least one or more PLT proteins;
a SHR protein, a SCR protein and a WIND1 protein;
a SHR protein, a WOX5 protein and at least one or more PLT proteins;
a SHR protein, a WOX5 protein and a WIND1 protein;
a SCR protein, a WOX5 protein and at least one or more PLT proteins;
a SCR protein, a WOX5 protein and a WIND1 protein;
a WOX5, at least one or more PLT proteins and a WIND1 protein;
a SHR protein, a SCR protein, a WOX5 protein and at least one or more PLT proteins;
a SHR protein, a SCR protein, a WOX5 protein and a WIND1 protein;
a SHR protein, a SCR protein, at least one or more PLT proteins and a WIND1 protein
a SHR protein, a WOX5 protein, at least one or more PLT proteins, and a WIND1 protein;
a SCR protein, a WOX5 protein, at least one or more PLT proteins, a WIND1 protein; and
a SHR protein, a SCR protein, a WOX5 protein, at least one or more PLT proteins, a WIND1 protein.

Preferably, the expression of the proteins listed above is transiently introduced or increased in the plant cell.

Preferably the one or more PLT proteins are selected from the group consisting of PLT1, PLT2, PLT3, PLT4 PLT5 and PLT7, preferably the selected PLT proteins include at least one or more of PLT1, PLT4 and PLT5. Preferably the one or more PLT proteins is PLT1 or at least PLT1. Preferably the PLT proteins are PLT1, PLT4 and PLT5.

In addition to each of these combinations listed above, the expression of an endogenous RBR protein can be decreased in the plant cell. Preferably, the expression of the RBR protein is transiently decreased.

In a particularly preferred embodiment, the invention pertains to a method for regenerating a plant cell, preferably a shoot from a plant cell, wherein the method comprises the step of altering in the plant cell the expression of at least one of the following combinations of proteins:
increasing expression of a WOX5 protein and a PLT1 protein
increasing expression of a WIND1 protein, a WOX5 protein and a PLT1 protein;
increasing expression of a SHR protein, a SCR protein, a WOX5 protein, a PLT1, PLT4 and PLT5 protein; and
increasing expression of a WIND1 protein, and a SHR protein, a SCR protein, a WOX5 protein, a PLT1, PLT4 and PLT5 protein, and downregulation of a RBR protein, preferably transient downregulation of the RBR protein.

Preferably, the introduced or increased expression of the proteins listed above is transiently introduced or increased in the plant cell.

In an embodiment, the method comprises a step of allowing the plant cell to regenerate, preferably into shoot.

In an embodiment, the proteins in the combination of proteins are derived from different families, e.g. derived from 2, 3, 4, 5 or 6 different families. The first family includes RBR, the second family includes WIND1, the third family includes SHR, the fourth family includes SCR, the fifth family includes WOX5 and the sixth family includes the PLT proteins, in particular the sixth family includes PLT1, PLT2, PLT3, PLT4, PLT5 and PLT7. The skilled person understands that other family members may be equally suitable in the method of the invention.

In an embodiment, the invention pertains to a method for regenerating a plant cell, preferably a shoot from a plant cell, comprising the steps of:
a1) introducing or increasing in the plant cell the expression of a WOX5 protein and at least one PLT protein. Preferably, the at least one PLT protein is selected from the group consisting of PLT1, PLT2, PLT3, PLT4, PLT5 and PLT7. Preferably, the at least one PLT protein is PLT1. Preferably, the expression of at least one of the WOX5 protein and the at least one PLT protein is transiently introduced or increased and
a2) optionally decreasing in the plant cell the expression of an endogenous RBR protein, wherein preferably, the expression of the RBR protein is transiently decreased; and
b) allowing the plant cell to regenerate, preferably into the shoot.

In an embodiment, the invention pertains to a method for regenerating a plant cell, preferably a shoot from a plant cell, comprising the steps of:
a1) introducing or increasing in the plant cell the expression of a WOX5 protein, at least one PLT protein and WIND1. Preferably, the at least one PLT protein is selected from the group consisting of PLT1, PLT2, PLT3, PLT4, PLT5 and PLT7. Preferably, the at least one PLT protein is PLT1. Preferably, the expression of at least one of the WOX5 protein, the WIND1 protein and the at least one PLT protein is transiently introduced or increased and
a2) optionally decreasing in the plant cell the expression of an endogenous RBR protein, wherein preferably, the expression of the RBR protein is transiently decreased; and
b) allowing the plant cell to regenerate, preferably into the shoot.

In an embodiment, the invention pertains to a method for regenerating a plant cell, preferably a shoot from a plant cell, comprising the steps of:
a1) introducing or increasing in the plant cell the expression of a SHR protein, a SCR protein, a WOX5 protein and at least one PLT protein. Preferably, the at least one PLT protein is selected from the group consisting of PLT1, PLT2, PLT3, PLT4, PLT5 and PLT7. Preferably, the expression of at least one of the SHR protein, the SCR protein, the WOX5 protein and the at least one PLT protein is transiently introduced or increased and
a2) optionally decreasing in the plant cell the expression of an endogenous RBR protein, wherein preferably, the expression of the RBR protein is transiently decreased; and
b) allowing the plant cell to regenerate, preferably into the shoot.

In a further embodiment the invention concerns a method for regenerating a plant cell, preferably a shoot from a plant cell comprising the steps of:
a1) introducing or increasing in the plant cell the expression of a SHR protein, a SCR protein, a WOX5 protein and at least two PLT proteins. Preferably, the at least two PLT proteins are selected from the group consisting of PLT1, PLT2, PLT3, PLT4, PLT5 and PLT7. Preferably, the expression of at least one of the SHR protein, the SCR protein, the WOX5 protein and at least one of the two PLT proteins, or both PLT proteins, is transiently introduced or increased;
a2) optionally decreasing in the plant cell the expression of an endogenous RBR protein, wherein preferably, the expression of the RBR protein is transiently decreased; and
b) allowing the plant cell to regenerate, preferably into the shoot.

In another embodiment, the invention pertains to a method for generating a plant cell, preferably regenerating a shoot from a plant cell, wherein the method comprises the steps of
a1) introducing or increasing in the plant cell the expression of a SHR protein, a SCR protein, a WOX5 protein and at least three PLT proteins. Preferably, the at least three PLT proteins are selected from the group consisting of PLT1, PLT2, PLT3, PLT4, PLT5 and PLT7. Preferably the at least three selected PLT proteins include at least one or more of PLT1, PLT4 and PLT5. Preferably, the at least three selected PLT proteins are PLT1, PLT4 and PLT5. Preferably, the expression of at least one of the SHR protein, the SCR protein, the WOX5 protein and at least one of PLT proteins selected from the group consisting of PLT1, PLT2, PLT3, PLT4, PLT5 and PLT7 is transiently introduced or increased. Preferably, the expression of at least one of the SHR protein, the SCR protein, the WOX5 protein, the PLT1 protein, PLT4 protein and PLT5 protein is transiently introduced or increased;
a2) optionally decreasing in the plant cell the expression of an endogenous RBR protein, wherein preferably, the expression of the RBR protein is transiently decreased; and
b) allowing the plant cell to regenerate, preferably into a shoot.

In another embodiment, the invention pertains to a method for generating a plant cell, preferably regenerating a shoot from a plant cell, wherein the method comprises the steps of
a1) introducing or increasing in the plant cell the expression of a SHR protein, a SCR protein, a WOX5 protein, a WIND1 protein and at least three PLT proteins. Preferably, the at least three PLT proteins are selected from the group consisting of PLT1, PLT2, PLT3, PLT4, PLT5 and PLT7. Preferably the at least three selected PLT proteins include at least one or more of PLT1, PLT4 and PLT5. Preferably, the at least three selected PLT proteins are PLT1, PLT4 and PLT5. Preferably, the expression of at least one of the SHR protein, the SCR protein, the WOX5 protein, the WIND1 protein and at least one of PLT proteins selected from the group consisting of PLT1, PLT2, PLT3, PLT4, PLT5 and PLT7 is transiently introduced or increased. Preferably, the expression of at least one of the SHR protein, the SCR protein, the WOX5 protein, the WIND1, the PLT1 protein, PLT4 protein and PLT5 protein is transiently introduced or increased;
a2) optionally decreasing in the plant cell the expression of an endogenous RBR protein, wherein preferably, the expression of the RBR protein is transiently decreased; and
b) allowing the plant cell to regenerate, preferably into a shoot.

Proteins for Use in the Method of the Invention

The combination of proteins for use in the invention includes at least one of SHR, SCR, WOX5, PLT1, PLT2, PLT3, PLT4, PLT5, PLT7, RBR, and WIND1. Preferably, the combination of proteins for use in the method includes at least WOX5 and a PLT protein selected from the group consisting of PLT1, PLT2, PLT3, PLT4, PLT5 and PLT7.

Particularly preferred combinations of proteins comprise at least or at most:
a WOX5 protein and a PLT1 protein
a WIND1 protein, a WOX5 protein and a PLT1 protein;
a SHR protein, a SCR protein, a WOX5 protein, a PLT1, PLT4 and PLT5 protein; and
a WIND1 protein, and a SHR protein, a SCR protein, a WOX5 protein, a PLT1, PLT4 and PLT5 protein and a RBR protein,

Preferably the proteins of the combinations of proteins have an induced or increased expression, with the exception of RBR. Preferably, the RBR protein has a decreased expression.

The amino acid sequence of the SHR protein can have at least about 50%, 55%, 60%, 65%, 70%, 75%, 80%, 85%, 90%, 95%, 98%, 99% or 100% sequence identity with SEQ ID NO: 1. SEQ ID NO: 1 is the Arabidopsis thaliana SHR protein (see Table 5 for an overview of all SEQ ID NOs used herein). In one embodiment, the SHR amino acid sequence is or is derived from AT4G37650, a homolog thereof, or a sequence having at least about 50%, 55%, 60%, 65%, 70%, 75%, 80%, 85%, 90%, 95%, 98%, 99% or 100% sequence identity with AT4G37650 or its homolog.

The amino acid sequence of the SCR protein can have at least about 50%, 55%, 60%, 65%, 70%, 75%, 80%, 85%, 90%, 95%, 98%, 99% or 100% sequence identity with SEQ ID NO: 2. SEQ ID NO: 2 is the Arabidopsis thaliana SCR protein. In one embodiment, the SCR amino acid sequence is or is derived from AT3G54220, a homolog thereof, or a sequence having at least about 50%, 55%, 60%, 65%, 70%, 75%, 80%, 85%, 90%, 95%, 98%, 99% or 100% sequence identity with AT3G54220 or its homolog.

The amino acid sequence of the WOX5 protein can have at least about 50%, 55%, 60%, 65%, 70%, 75%, 80%, 85%, 90%, 95%, 98%, 99% or 100% sequence identity with SEQ ID NO: 3. SEQ ID NO: 3 is the Arabidopsis thaliana WOX5 protein. In one embodiment, the WOX5 amino acid sequence is or is derived from AT3G11260, a homolog thereof, or a sequence having at least about 50%, 55%, 60%, 65%, 70%, 75%, 80%, 85%, 90%, 95%, 98%, 99% or 100% sequence identity with AT3G11260 or its homolog.

The amino acid sequence of the PLT1 protein can have at least about 50%, 55%, 60%, 65%, 70%, 75%, 80%, 85%, 90%, 95%, 98%, 99% or 100% sequence identity with SEQ ID NO: 4. SEQ ID NO: 4 is the Arabidopsis thaliana PLT1 protein. In one embodiment, the PLT1 amino acid sequence is or is derived from AT3G20840, a homolog thereof, or a sequence having at least about 50%, 55%, 60%, 65%, 70%, 75%, 80%, 85%, 90%, 95%, 98%, 99% or 100% sequence identity with AT3G20840 or its homolog.

The amino acid sequence of the PLT2 protein can have at least about 50%, 55%, 60%, 65%, 70%, 75%, 80%, 85%, 90%, 95%, 98%, 99% or 100% sequence identity with SEQ ID NO: 5. SEQ ID NO: 5 is the Arabidopsis thaliana PLT2 protein. In one embodiment, the PLT2 amino acid sequence is or is derived from AT1G51190, a homolog thereof, or a sequence having at least about 50%, 55%, 60%, 65%, 70%, 75%, 80%, 85%, 90%, 95%, 98%, 99% or 100% sequence identity with AT1G51190 or its homolog.

The amino acid sequence of the PLT3 protein can have at least about 50%, 55%, 60%, 65%, 70%, 75%, 80%, 85%, 90%, 95%, 98%, 99% or 100% sequence identity with SEQ ID NO: 6. SEQ ID NO: 6 is the Arabidopsis thaliana PLT3 protein. In one embodiment, the PLT3 amino acid sequence is or is derived from AT5G10510, a homolog thereof, or a sequence having at least about 50%, 55%, 60%, 65%, 70%, 75%, 80%, 85%, 90%, 95%, 98%, 99% or 100% sequence identity with AT5G10510 or its homolog.

The amino acid sequence of the PLT4 protein can have at least about 50%, 55%, 60%, 65%, 70%, 75%, 80%, 85%, 90%, 95%, 98%, 99% or 100% sequence identity with SEQ ID NO: 7. SEQ ID NO: 7 is the Arabidopsis thaliana PLT4 protein. In one embodiment, the PLT4 amino acid sequence is or is derived from AT5G17430, a homolog thereof, or a sequence having at least about 50%, 55%, 60%, 65%, 70%, 75%, 80%, 85%, 90%, 95%, 98%, 99% or 100% sequence identity with AT5G17430 or its homolog.

The amino acid sequence of the PLT5 protein can have at least about 50%, 55%, 60%, 65%, 70%, 75%, 80%, 85%, 90%, 95%, 98%, 99% or 100% sequence identity with SEQ ID NO: 8. SEQ ID NO: 8 is the Arabidopsis thaliana PLT5 protein. In one embodiment, the PLT5 amino acid sequence is or is derived from AT5G57390, a homolog thereof, or a sequence having at least about 50%, 55%, 60%, 65%, 70%, 75%, 80%, 85%, 90%, 95%, 98%, 99% or 100% sequence identity with AT5G57390 or its homolog.

The amino acid sequence of the PLT7 protein can have at least about 50%, 55%, 60%, 65%, 70%, 75%, 80%, 85%, 90%, 95%, 98%, 99% or 100% sequence identity with SEQ ID NO: 30. SEQ ID NO:30 is the Arabidopsis thaliana PLT7 protein. In one embodiment, the PLT7 amino acid sequence is or is derived from AT5G65510, a homolog thereof, or a sequence having at least about 50%, 55%, 60%, 65%, 70%, 75%, 80%, 85%, 90%, 95%, 98%, 99% or 100% sequence identity with AT5G65510 or its homolog.

The amino acid sequence of the WIND1 protein can have at least about 50%, 55%, 60%, 65%, 70%, 75%, 80%, 85%, 90%, 95%, 98%, 99% or 100% sequence identity with SEQ ID NO: 28. SEQ ID NO: 28 is the Arabidopsis thaliana WIND1 protein. In one embodiment, the WIND1 amino acid sequence is or is derived from AT1G78080, a homolog thereof, or a sequence having at least about 50%, 55%, 60%, 65%, 70%, 75%, 80%, 85%, 90%, 95%, 98%, 99% or 100% sequence identity with AT1G78080 or its homolog.

The amino acid sequence of the RBR protein can have at least about 50%, 55%, 60%, 65%, 70%, 75%, 80%, 85%, 90%, 95%, 98%, 99% or 100% sequence identity with SEQ ID NO: 17. SEQ ID NO: 17 is the Arabidopsis thaliana RBR protein. In one embodiment, the RBR amino acid sequence is or is derived from AT3G12280, a homolog thereof, or a sequence having at least about 50%, 55%, 60%, 65%, 70%, 75%, 80%, 85%, 90%, 95%, 98%, 99% or 100% sequence identity with AT3G12280 or its homolog.

The proteins as defined herein can further comprise a tag, such as but not limited to a T7 tag (e.g. T7 tag having at least about 50%, 55%, 60%, 65%, 70%, 75%, 80%, 85%, 90%, 95%, 98%, 99% or 100% sequence identity with SEQ ID NO: 53), a Myc tag (e.g. Myc-tag having a sequence of at least about 50%, 55%, 60%, 65%, 70%, 75%, 80%, 85%, 90%, 95%, 98%, 99% or 100% sequence identity with SEQ ID NO: 43), FLAG-tag (e.g. FLAG-tag having a sequence of at least about 50%, 55%, 60%, 65%, 70%, 75%, 80%, 85%, 90%, 95%, 98%, 99% or 100% sequence identity with SEQ ID NO: 46), a V5-tag (e.g. V5 tag having at least about 50%, 55%, 60%, 65%, 70%, 75%, 80%, 85%, 90%, 95%, 98%, 99% or 100% sequence identity with SEQ ID NO: 50), or a His-tag. Preferably, the tag is located at the C-terminus of the protein, before the original stop codon.

Nucleic Acids for Use in the Method of the Invention

In one embodiment, the SHR protein is encoded by a nucleotide sequence having at least about 50%, 55%, 60%, 65%, 70%, 75%, 80%, 85%, 90%, 95%, 98%, 99% or 100% sequence identity with SEQ ID NO: 9. The nucleotide sequence encoding the SHR protein can be, or can be derived from the gene AT4G37650, a homolog thereof or a sequence having at least about 50%, 55%, 60%, 65%, 70%, 75%, 80%, 85%, 90%, 95%, 98%, 99% or 100% sequence identity with AT4G37650 or its homolog. The percentage identity can be determined over the full length of the genomic sequence. Alternatively the percentage identity can be determined over the full length of the coding sequence of the gene.

Examples of homologs include Brachypodium distachyon (Purple false brome) (BRADI1G23060), Glycine max (Soybean) (GLYMA01G40180, GLYMA05G22460, GLYMA11G05110, GLYMA11G23690 or GLYMA17G17400), Oryza sativa (Rice) (SHR1 and SHR2), Physcomitrella patens (Moss) (PHYPADRAFT_14911 and PHYPADRAFT_22633), Populus trichocarpa (Black Cottonwood) (POPTR_0012S06430G), Solanum lycopersicum (Tomato) (SOLYC02G092370.1), Sorghum bicolor (Sorghum) (SB01G031720 and SB02G037890) and Vitis vinifera (Grape) VIT_0750129G00030.

In one embodiment, the SCR protein is encoded by a nucleotide sequence having at least about 50%, 55%, 60%, 65%, 70%, 75%, 80%, 85%, 90%, 95%, 98%, 99% or 100% sequence identity with SEQ ID NO: 10. The nucleotide sequence encoding the SCR protein can be, or can be derived from the gene AT3G54220, a homolog thereof or a sequence having at least about 50%, 55%, 60%, 65%, 70%, 75%, 80%, 85%, 90%, 95%, 98%, 99% or 100% sequence identity with AT3G54220 or its homolog. The percentage identity can be determined over the full length of the genomic sequence. Alternatively the percentage identity can be determined over the full length of the coding sequence of the gene.

Examples of homologs include Brachypodium distachyon (Purple false brome) (BRADI4G44090), Glycine max (Soybean) (GLYMA09G40620 and GLYMA18G45220), Oryza sativa (Rice) (SCR1 and SCR2), Physcomitrella patens (Moss) (PHYPADRAFT_150910, PHYPADRAFT_22273, PHYPADRAFT_42008 and PHYPADRAFT_65480), Populus trichocarpa (Black Cottonwood) (POPTR_0006S11500G and POPTR_0016S15060G), Solanum lycopersicum (Tomato) SOLYC10G074680.1, Sorghum bicolor (Sorghum) SB05G001500 and Vitis vinifera (Grape) VIT_08S0056G00050.

In one embodiment, the WOX5 protein is encoded by a nucleotide sequence having at least about 50%, 55%, 60%, 65%, 70%, 75%, 80%, 85%, 90%, 95%, 98%, 99% or 100% sequence identity with SEQ ID NO: 11. The nucleotide sequence encoding the WOX5 protein can be, or can be derived from the gene AT3G11260, a homolog thereof or a sequence having at least about 50%, 55%, 60%, 65%, 70%, 75%, 80%, 85%, 90%, 95%, 98%, 99% or 100% sequence identity with AT3G11260 or its homolog. The percentage identity can be determined over the full length of the genomic sequence. Alternatively the percentage identity can be determined over the full length of the coding sequence of the gene.

Examples of homologs include Arabidopsis thaliana (Thale cress) (AT5G05770.1), Brachypodium distachyon (Purple false brome) (BRADI2G55270), Glycine max (Soybean) (GLYMA02G42200), Oryza sativa (Rice) (WOX9), Populus trichocarpa (Black Cottonwood) (POPTR_0008S06560 G and POPTR_0010S19950G), Solanum lycopersicum (Tomato) (SOLYC03G096300.2), Sorghum bicolor (Sorghum) (SB03G040210) and Vitis vinifera (Grape) (VIT_13S0019G03460).

In one embodiment, the PLT1 protein is encoded by a nucleotide sequence having at least about 50%, 55%, 60%, 65%, 70%, 75%, 80%, 85%, 90%, 95%, 98%, 99% or 100% sequence identity with SEQ ID NO: 12. The nucleotide sequence encoding the PLT1 protein can be, or can be derived from the gene AT3G20840, a homolog thereof or a sequence having at least about 50%, 55%, 60%, 65%, 70%, 75%, 80%, 85%, 90%, 95%, 98%, 99% or 100% sequence identity with AT3G20840 or its homolog. The percentage identity can be determined over the full length of the genomic sequence. Alternatively the percentage identity can be determined over the full length of the coding sequence of the gene.

Examples of homologs include Arabidopsis thaliana (Thale cress) (AT1G51190.1), Glycine max (Soybean) (GLYMA11G14040 and GLYMA12G06010), Populus trichocarpa (Black Cottonwood) (POPTR_0001S05580 G and POPTR_0003S20470G), Solanum lycopersicum (Tomato) (SOLYC11G061750.1), and Vitis vinifera (Grape) (VIT_06S0004G01800)

In one embodiment, the PLT2 protein is encoded by a nucleotide sequence having at least about 50%, 55%, 60%, 65%, 70%, 75%, 80%, 85%, 90%, 95%, 98%, 99% or 100% sequence identity with SEQ ID NO: 13. The nucleotide sequence encoding the PLT2 protein can be, or can be derived from the gene AT1G51190, a homolog thereof or a sequence having at least about 50%, 55%, 60%, 65%, 70%, 75%, 80%, 85%, 90%, 95%, 98%, 99% or 100% sequence identity with AT1G51190 or its homolog. The percentage identity can be determined over the full length of the genomic sequence. Alternatively the percentage identity can be determined over the full length of the coding sequence of the gene.

Examples of homologs include Arabidopsis thaliana (Thale cress) (AT3G20840.1), Glycine max (Soybean) (GLYMA11G14040 and GLYMA12G06010), Populus trichocarpa (Black Cottonwood) (POPTR_0001S05580G and POPTR_0003S20470G), Solanum lycopersicum (Tomato SOLYC11G061750.1) and Vitis vinifera (Grape) (VIT_06S0004G01800)

In one embodiment, the PLT3 protein is encoded by a nucleotide sequence having at least about 50%, 55%, 60%, 65%, 70%, 75%, 80%, 85%, 90%, 95%, 98%, 99% or 100% sequence identity with SEQ ID NO: 14. The nucleotide sequence encoding the PLT3 protein can be, or can be derived from the gene AT5G10510, a homolog thereof or a sequence having at least about 50%, 55%, 60%, 65%, 70%, 75%, 80%, 85%, 90%, 95%, 98%, 99% or 100% sequence identity with AT5G10510 or its homolog. The percentage identity can be determined over the full length of the genomic sequence. Alternatively the percentage identity can be determined over the full length of the coding sequence of the gene.

In one embodiment, the PLT4 protein is encoded by a nucleotide sequence having at least about 50%, 55%, 60%, 65%, 70%, 75%, 80%, 85%, 90%, 95%, 98%, 99% or 100% sequence identity with SEQ ID NO: 15. The nucleotide sequence encoding the PLT4 protein can be, or can be derived from the gene AT5G17430, a homolog thereof or a sequence having at least about 50%, 55%, 60%, 65%, 70%, 75%, 80%, 85%, 90%, 95%, 98%, 99% or 100% sequence identity with AT5G17430 or its homolog. The percentage identity can be determined over the full length of the genomic sequence. Alternatively the percentage identity can be determined over the full length of the coding sequence of the gene.

Examples of homologs include Brachypodium distachyon (Purple false brome) (BRADI3G48697 and BRADI5G14960), Glycine max (Soybean) (GLYMA09G38370 and GLYMA10G31440), Oryza sativa (Rice) (OSJNBB0116K07.8), Populus trichocarpa (Black Cottonwood) (POPTR_0008S07610G and POPTR_0010S18840G), Solanum lycopersicum (Tomato) (SOLYC11G008560.1) and Sorghum bicolor (Sorghum) (SB04G025960)

In one embodiment, the PLT5 protein is encoded by a nucleotide sequence having at least about 50%, 55%, 60%, 65%, 70%, 75%, 80%, 85%, 90%, 95%, 98%, 99% or 100% sequence identity with SEQ ID NO: 16. The nucleotide sequence encoding the PLT5 protein can be, or can be derived from the gene AT5G57390, a homolog thereof or a sequence having at least about 50%, 55%, 60%, 65%, 70%, 75%, 80%, 85%, 90%, 95%, 98%, 99% or 100% sequence identity with AT5G57390 or its homolog. The percentage identity can be determined over the full length of the genomic sequence. Alternatively the percentage identity can be determined over the full length of the coding sequence of the gene.

Examples of homologs include Arabidopsis thaliana (Thale cress) (AT4G37750.1), Brachypodium distachyon (Purple false brome) (BRADI1G07290), Glycine max (Soybean) (GLYMA0041S50, GLYMA01G40380, GLYMA02G31035, GLYMA05G22970, GLYMA06G05170, GLYMA11G04910, GLYMA14G10130 and GLYMA17G17010), Oryza sativa (Rice) (OSJNBA0072F13.9), Physcomitrella patens (Moss) PHYPADRAFT_127673, PHYPADRAFT_127688, PHYPADRAFT_136724 and PHYPADRAFT_189336), Populus trichocarpa (Black Cottonwood) (POPTR_0002S11550G, POPTR_0005S19220G, POPTR_0007S14690G and POPTR_0014S01260G), Solanum lycopersicum (Tomato) (SOLYC02G092050.2, SOLYC03G123430.2 and SOLYC04G077490.2), Sorghum bicolor (Sorghum) (SB01G006830) and Vitis vinifera (Grape) (VIT_07S0151G00440 and VIT_18S0001G08610).

In one embodiment, the PLT7 protein is encoded by a nucleotide sequence having at least about 50%, 55%, 60%, 65%, 70%, 75%, 80%, 85%, 90%, 95%, 98%, 99% or 100% sequence identity with SEQ ID NO: 19. The nucleotide sequence encoding the PLT7 protein can be, or can be derived from the gene AT5G65510, a homolog thereof or a sequence having at least about 50%, 55%, 60%, 65%, 70%, 75%, 80%, 85%, 90%, 95%, 98%, 99% or 100% sequence identity with AT5G65510 or its homolog. The percentage identity can be determined over the full length of the genomic sequence. Alternatively the percentage identity can be determined over the full length of the coding sequence of the gene.

Examples of homologs include Arabidopsis thaliana (Thale cress) (AT5G10510.2), Brachypodium distachyon (Purple false brome) (BRADI1G31337), Glycine max (Soybean) (GLYMA01G02760, GLYMA08G38190, GLYMA09G33241 and GLYMA18G29400), Oryza sativa (Rice) (P0677B10.15), Populus trichocarpa (Black Cottonwood) (POPTR_0007S14210G), Solanum lycopersicum (Tomato) (SOLYC05G051380.2 and SOLYC11G010710.1), Sorghum bicolor (Sorghum) (SB10G026150) and Vitis vinifera (Grape) (VIT_00S0772G00020 and VIT_00S1291G00010).

In one embodiment, the WIND1 protein is encoded by a nucleotide sequence having at least about 50%, 55%, 60%, 65%, 70%, 75%, 80%, 85%, 90%, 95%, 98%, 99% or 100% sequence identity with SEQ ID NO: 29. The nucleotide sequence encoding the WIND1 protein can be, or can be derived from the gene AT1G78080, a homolog thereof or a sequence having at least about 50%, 55%, 60%, 65%, 70%, 75%, 80%, 85%, 90%, 95%, 98%, 99% or 100% sequence identity with AT1G78080 or its homolog. The percentage identity can be determined over the full length of the genomic sequence. Alternatively the percentage identity can be determined over the full length of the coding sequence of the gene.

Examples of homologs include Arabidopsis thaliana (Thale cress) (AT1G22190.1, AT1G36060.1, AT1G64380.1, AT2G20880.1, AT2G22200.1, AT4G13620.1, AT4G28140.1, AT4G39780.1 and AT5G65130.1), Brachypodium distachyon (Purple false brome) (BRADI1G45470), Glycine max (Soybean) (GLYMA01G43450, GLYMA05G31370, GLYMA06G45010, GLYMA08G14600, GLYMA10G33700, GLYMA11G02050, GLYMA12G12270, GLYMA12G33020, GLYMA13G01930, GLYMA13G37451, GLYMA14G34590, GLYMA18G02170 and GLYMA20G33890), Oryza sativa (Rice) (OS06G0222400 and P0516A04.31), Physcomitrella patens (Moss) (PHYPADRAFT_142112 and PHYPADRAFT_151367), Populus trichocarpa (Black Cottonwood) (POPTR_0001S10540G, POPTR_0001S32250G, POPTR_0002S09480G, POPTR_0003S13910G, POPTR_0005S07900G, POPTR_0005S16690G, POPTR_0007S05690G, POPTR_0013S13920G, POPTR_0017S08250G and POPTR_0019S13330G), Solanum lycopersicum (Tomato) (DREB3, SOLYC04G054910.2, SOLYC07G054220.1, SOLYC08G082210.2, SOLYC09G091950.1, SOLYC12G013660.1 and SOLYC12G056980 0.1), Sorghum bicolor (Sorghum) (SB01G044410, SB02G023230, SB07G020090, SB08G007411 and SB10G007780) and Vitis vinifera (Grape) (VIT_00S0662G00030, VIT_00S0662G00040, VIT_02S0025G01360, VIT_05S0029G00140, VIT_12S0059G00280, VIT_18 S0001G05250 and VIT_19S0014G03180).

The sequence encoding the SHR, SCR, WOX5, PLT1, PLT2, PLT3, PLT4, PLT5, PLT7 or WIND1 protein is preferably codon-optimized for expression in plant cells, preferably codon-optimized for expression in the plant cell of the method of the invention, preferably codon-optimized for the species of the plant cell used in the method of the invention. As a non-limiting example, the overexpressed or de novo expressed protein as defined herein can be an endogenous protein while the sequence encoding this endogenous protein is an exogenous, codon-optimized, sequence. Alternatively, the codon-optimized sequence can encode a protein that is exogenous for the plant cell.

In an embodiment of the invention, the proteins having increased or introduced expression as defined herein are functional proteins. A SHR as defined herein is preferably fulfilling the same or similar function in a plant cell as the function of a protein having amino acid sequence of SEQ ID NO: 1 in Arabidopsis thaliana. A SCR as defined herein is preferably fulfilling the same or similar function in a plant cell as the function of a protein having amino acid sequence of SEQ ID NO: 2 in Arabidopsis thaliana. A WOX5 as defined herein is preferably fulfilling the same or similar function in a plant cell as the function of a protein having amino acid sequence of SEQ ID NO: 3 in Arabidopsis thaliana. A PLT1 as defined herein is preferably fulfilling the same or similar function in a plant cell as the function of a protein having amino acid sequence of SEQ ID NO: 4 in Arabidopsis thaliana. A PLT2 as defined herein is preferably fulfilling the same or similar function in a plant cell as the function of a protein having amino acid sequence of SEQ ID NO: 5 in Arabidopsis thaliana. A PLT3 as defined herein is preferably fulfilling the same or similar function in a plant cell as the function of a protein having amino acid sequence of SEQ ID NO: 6 in Arabidopsis thaliana. A PLT4 as defined herein is preferably fulfilling the same or similar function in a plant cell as the function of a protein having amino acid sequence of SEQ ID NO: 7 in Arabidopsis thaliana. A PLT5 as defined herein is preferably fulfilling the same or similar function in a plant cell as the function of a protein having amino acid sequence of SEQ ID NO: 8 in Arabidopsis thaliana. A PLT7 as defined herein is preferably fulfilling the same or similar function in a plant cell as the function of a protein having amino acid sequence of SEQ ID NO: 30 in Arabidopsis thaliana. A WIND1 as defined herein is preferably fulfilling the same or similar function in a plant cell as the function of a protein having amino acid sequence of SEQ ID NO: 28 in Arabidopsis thaliana.

In one embodiment, the endogenous RBR protein is encoded by a nucleotide sequence having at least about 50%, 55%, 60%, 65%, 70%, 75%, 80%, 85%, 90%, 95%, 98%, 99% or 100% sequence identity with SEQ ID NO: 18. The nucleotide sequence encoding the RBR protein can be, or can be derived from, the gene AT3G12280, a homolog thereof or a sequence having at least about 50%, 55%, 60%, 65%, 70%, 75%, 80%, 85%, 90%, 95%, 98%, 99% or 100% sequence identity with AT3G12280 or its homolog. The percentage identity can be determined over the full length of the genomic sequence. Alternatively the percentage identity can be determined over the full length of the coding sequence of the gene.

Examples of homologs include Brachypodium distachyon (Purple false brome) (BRADI3G41630), Chlamydomonas reinhardtii (Chlamydomonas) (MAT3), Glycine max (Soybean) (GLYMA04G36700, GLYMA13G26170 and GLYMA15G36890), Oryza sativa (Rice) (RBR1), Physcomitrella patens (Moss) (PHYPADRAFT_88833, RBL1502 and RBR), Populus trichocarpa (Black Cottonwood) (RBL901), Solanum lycopersicum (Tomato) (SOLYC09G091280.2), Sorghum bicolor (Sorghum) (SB07G025760) and Vitis vinifera (Grape) (VIT_04S0008G02780).

In an embodiment of the invention, the RBR protein having a decreased expression is a functional protein. Hence, a RBR as defined herein is preferably fulfilling the same or similar function in a plant cell as the function of a protein having amino acid sequence of SEQ ID NO: 17 in Arabidopsis thaliana.

In one embodiment, the skilled person can use a sequence having have at least about 50%, 55%, 60%, 65%, 70%, 75%, 80%, 85%, 90%, 95%, 98%, 99% or 100% sequence identity with SEQ ID NO: 17 or SEQ ID NO: 18 to find a sequence, preferably genomic sequence, encoding an homologous RBR protein in a plant cell, preferably in the genome of a plant cell as defined herein. This sequence can subsequently be used target and downregulate the expression of an endogenous RBR protein, e.g. by using the RNAi machinery.

Transient Increased (SHR, SCR, WOX5, PLT1-PL5, PLT7, WIND1) or Decreased (RBR) Expression

In one embodiment, the expression of at least one of the SHR protein, the SCR protein, the WOX5 protein, the WIND1 protein and the expression of at least one, two, three, four, five or six PLT proteins selected from the group consisting of PLT1, PLT2, PLT3, PLT4, PLT5 and PLT7, is transiently introduced or increased. Optionally, additionally the expression of the endogenous RBR protein is transiently decreased.

In one embodiment the expression of at least WOX5 and a PLT protein selected from the group consisting of PLT1, PLT2, PLT3, PLT4, PLT5 and PLT7 is transiently introduced or increased. Preferably the expression of at least WOX5 and PLT1 is transiently introduced or increased. The expression of only WOX5 and PLT1 can be transiently introduced or increased.

In one embodiment the expression of at least WIND1, WOX5 and a PLT protein selected from the group consisting of PLT1, PLT2, PLT3, PLT4, PLT5 and PLT7 is transiently introduced or increased. Preferably the expression of at least WIND1, WOX5 and PLT1 is transiently introduced or increased. The expression of only WIND1, WOX5 and PLT1 can be transiently introduced or increased.

In one embodiment the expression of at least SHR, SCR, WOX5 and one, two or three PLT proteins selected from the group consisting of PLT1, PLT2, PLT3, PLT4, PLT5 and PLT7 is transiently introduced or increased. Preferably the expression of at least SHR, SCR, WOX5, PLT1, PLT4 and PLT5 is transiently introduced or increased. The expression of only SHR, SCR, WOX5, PLT1, PLT4 and PLT5 can be transiently introduced or increased.

In one embodiment the expression of at least WIND1, SHR, SCR, WOX5 and one, two or three PLT proteins selected from the group consisting of PLT1, PLT2, PLT3, PLT4, PLT5 and PLT7 is transiently introduced or increased. Preferably the expression of at least WIND1, SHR, SCR, WOX5, PLT1, PLT4 and PLT5 is transiently introduced or increased. The expression of only WIND1, SHR, SCR, WOX5, PLT1, PLT4 and PLT5 can be transiently introduced or increased. In addition, the expression of the endogenous RBR protein is transiently decreased.

Increased or decreased expression of the proteins as defined herein induces the regeneration of a plant cell, preferably shoot regeneration. The transient introduced or increased expression, and optionally the transient decreased expression, of the proteins as defined herein can be sequential or simultaneously. For example, the transient expression of a first protein or proteins of a combination of proteins as defined herein, such as e.g. WIND1, may occur before the transient expression of a subsequent protein or proteins as defined herein. The increased or introduced expression of these subsequent protein or proteins of the combination of proteins can be during or after the increased or introduced expression of the first protein or proteins.

Similarly, the transient decreased expression of a RBR protein as defined herein may occur before, during or after the transient introduced or increased expression of the protein or proteins as defined herein. Likewise, the increased or introduced expression of the protein or proteins can be before, during or after the decreased expression of the RBR protein.

Preferably, at one time period there is a simultaneous increased or introduced expression in the plant cell of at least two, three, four, five or six proteins selected from the group consisting of WIND1, SHR, SCR, WOX5, PLT1, PLT2, PLT3, PLT4, PLT5 and PLT7. Optionally the expression of the RBR protein is decreased concomitantly or during the same time period.

Preferably, at one time period there is a simultaneous increased or introduced expression in the plant cell of at least two, three, four, five or six proteins selected from the group consisting of WIND1, SHR, SCR, WOX5, PLT1, PLT4 and PLT5.

For example, at one time period there is simultaneous increased or introduced expression of at least WOX5 and PLT1, or simultaneous increased or introduced expression of at least WOX5, PLT1 and WIND1, or simultaneous increased or introduced expression of at least SHR, SCR, WOX5, PLT1, PLT4 and PLT5, or simultaneous increased or introduced expression of at least WIND1, SHR, SCR, WOX5, PLT1, PLT4 and PLT5. Optionally the expression of the RBR protein is decreased concomitantly or during the same time period.

Alternatively or in addition, the expression of a protein or proteins as defined herein may be transiently introduced or increased, and optionally decreased, before the introduced or increased expression of a subsequent protein or proteins. In one embodiment, the expression of WIND1 is transiently introduced or increased and optionally the expression of RBR is transiently decreased, before the introduced or increased expression of one or more of SHR, SCR, WOX5 and one or more PLT proteins as defined herein.

As a non-limiting example, the expression of WIND1 can be transiently increased or introduced before the transient increased or introduced expression of at least WOX5 and PLT1. The expression of WIND1 can be transiently increased or introduced before the transient increased or introduced expression of at least WOX5, PLT1, SHR, SCR, PLT4 and PLT5. The expression of RBR can be decreased simultaneously with the increased or introduced expression of WIND1.

The introduced or increased expression of WIND1, and optionally the decreased RBR expression, can occur before the expression of any of the other proteins as defined herein. The WIND1 expression levels, and optionally the RBR expression levels, may remain altered during the introduced or increased expression of the other proteins of the combination of proteins as defined herein. Alternatively the WIND1, and optionally RBR, expression levels may have returned to endogenous levels prior to the increased or introduced expression of the other proteins of the combination of proteins as defined herein.

The time period between introducing the altered WIND1, and optionally RBR, expression levels and inducing the altered expression levels of the other proteins of the composition of proteins as defined herein, is preferably at least about 0, 1, 2, 3, 4, 5, 6, 7, 8, 9, 10, 12 or at least about 14 days.

Time Period

The time period wherein the proteins as defined herein have an increased or introduced, or optionally decreased, expression (as specified herein for the particular proteins) in the plant cell is preferably a period that is sufficiently long to induce regeneration of the plant cell. However, maintaining the altered expression levels can hamper the further development of the regenerated plant cell into a plant. Therefore in a preferred embodiment, the altered expression of the at least one, two, three, four, five or six proteins is transient. Expression levels of the proteins as defined herein can be returned to endogenous levels, i.e. to protein levels prior to the increased, introduced or decreased expression, preferably before the protein or proteins induce uncontrolled meristem formation. Returning the protein levels to endogenous levels preferably initiates tissue differentiation.

In one embodiment, the time period wherein a combination of proteins as defined herein has an increased, introduced or decreased expression in the plant cell is at least about 1, 2, 3, 4, 5, 6, 7, 8, 9, 10, 11, 12, 13, 14, 15, 16, 17, 18, 19, 20, 25, 30, 35, 40, 45 or at least about 50 days. The time period can be at least about 1, 2, 3, 4, 5, 6, 7, 8, 9 or at least about 10 weeks. After this time period the protein levels of at least one protein, preferably all proteins, can return to endogenous levels. Hence, the time period can be less than about 100, 95, 90, 85, 80, 75, 70, 65, 60, 55, 50, 45, 40, 35, 30, 25, 20 or less that about 15 days. The time period can be less than about 20, 15, 12, 10, 8, 7, 6, 5, 4, 3 or less than about 2 weeks. The period wherein the protein or proteins as defined herein have an increased or introduced expression can be about 0.5-10 weeks, 1-8 weeks, 1-6 weeks, 1-4 weeks, about 1-3 weeks or about 2 weeks.

Preferably, at the same time period as defined herein above wherein at least one, two, three, four, five or six proteins selected from the group consisting of WIND1, SHR, SCR, WOX5, PLT1, PLT2, PLT3, PLT4 and PLT5, have an increased or introduced expression in the plant cell, preferably the same time period wherein each of the proteins of a combination of proteins as defined herein have an increased or introduced expression, the plant cell also has a decreased expression of an endogenous RBR protein.

The time period for introducing, increasing and optionally decreasing the expression of the protein or proteins as defined herein can be dependent on the type of plant cell and/or the regeneration conditions, and the suitable time period can be determined using conventional means known in the art. As a non-limiting example, expression of the proteins can be introduced or increased and optionally decreased as defined herein and the morphology of the regenerating plant can be monitored. Protein levels can be returned to normal, or endogenous levels, shortly before or after regeneration. For example, the introduced or increased, and optionally decreased, expression of the proteins of the protein combinations as defined herein can be returned to endogenous levels about 2, 4, 6, 8 or 10 weeks or about 2, 4, 6, 8 or 10 months after regeneration, e.g. after the appearance of the first shoot.

Transiently Introduced or Increased Expression

In an embodiment, the expression of at least one of the WOX5, WIND1, SHR, SCR, PLT1, PLT2, PLT3, PLT4, PLT5 and PLT7 is introduced or increased in a plant cell. Such increase may be due to increased expression of endogenous proteins (i.e. via mutations in the endogenous gene encoding these protein—the regulatory or encoding sequences—that result in increased expression of functional proteins or via mutations in the regulatory or encoding sequences that result in increased protein function) or via transgenic introduction of constructs encoding the protein sequences. The introduced or increased expression is preferably a transient expression. Hence, the expression is introduced or increased during a certain time period as defined herein above, preferably the expression is introduced or increased only during a certain time period. Transient increased or introduced expression can be accomplished using any suitable means known in the art. For example, expression of the previously introduced protein or proteins can be knocked out, e.g. using targeted mutagenesis or RNAi.

Alternatively, transient increased expression can be accomplished by transient introduction of one or more of the proteins as defined herein into the plant cell. Alternatively or in addition, transient expression is achieved by introducing one or more nucleic acid constructs into a plant cell, wherein the nucleic acid construct comprises a coding sequence encoding one or more of the proteins as defined herein, and wherein said coding sequence is operably linked to a regulatory element, e.g. a constitutive or tissue-specific promoter. It is understood herein that a “constitutive” promoter is a promoter that is active in most tissues under most physiological and developmental conditions. A “tissue specific” promoter is only active in specific types of tissues or cells.

Alternatively or in addition, transient expression of the protein or proteins as defined herein is accomplished by transient activation of their gene expression. Hence, the expression of at least one of the WOX5, WIND1, SHR, SCR, PLT1, PLT2, PLT3, PLT4, PLT5 and PLT7 proteins, preferably a combination of proteins as defined herein, is transiently introduced or increased by transient activation of their expression.

Transient activation of expression can be achieved by placing one or more of the sequences encoding a protein as defined herein under the control of an inducible promoter. An “inducible” promoter is defined herein as a promoter that is physiologically (e.g. by external application of certain compounds) or developmentally regulated. Preferably, at least one, two, three, four, five, six, or at least seven genes encoding the WOX5, WIND1, SHR, SCR, PLT1, PLT2, PLT3, PLT4, PLT5 or the PLT7 protein, respectively, are placed under the control of an inducible promoter. The expression of the proteins as defined herein can be controlled by the same type of inducible promoter. Alternatively, the expression of the different proteins can be controlled by different types of inducible promoters.

The inducible promoter can be placed upstream of, and operably linked to, one or more endogenous genes, wherein the endogenous gene encodes a protein as defined herein, e.g. upstream of an endogenous gene encoding WOX5, WIND1, SHR, SCR, PLT1, PLT2, PLT3, PLT4, PLT5 or PLT7. Preferred examples of inducible promoters are described herein below in the second aspect

Alternatively or in addition, the plant cell may be stably transformed with a construct, wherein the expression of at least one of WOX5, WIND1, SHR, SCR PLT1, PLT2, PLT3, PLT4 or PLT5 is controlled by an inducible promoter. Preferably the plant cell is transformed with a construct as described below in the second aspect. Preferably, the plant cell is stably transformed with the expression construct, or constructs as defined herein in the second aspect.

As a non-limiting example, a transactivator can bind to the inducible promoter and subsequently induces the transcription of one of the proteins as defined herein. Such transactivators may first be activated by binding to a specific compound (an inducer). Alternatively, binding of the transactivator to the inducible promoter can be repressed when a specific compound (a repressor) is present. Preferably, the transactivator for use in the method of the current invention is activated upon binding tot a specific compound (an inducer). Preferably, the inducer is an inducer as described herein below in the second aspect.

Transiently Decreased Expression

In combination with the increased or introduced expression of at least one of the proteins as defined herein above, in an embodiment the expression of a RBR protein is decreased in a plant cell, preferably the expression is transiently decreased. It is herein understood that the decreased expression of an RBR protein is a decreased expression of the endogenous protein. Such decreased expression may be due to one or more mutations of the endogenous gene, i.e. mutations in the regulatory sequence (such as the promoter) or encoding sequence, that result in decreased expression of the endogenous functional protein, or via transgenic introduction of constructs encoding repressors.

The expression is preferably decreased during a certain time period as defined herein above, preferably the expression is decreased only during a certain time period as defined herein above. Transient decreased expression can be accomplished using any suitable means known in the art. For example, transient decreased expression can be accomplished by introduction of an small RNA transcript into a cell (e.g. a miRNA or an siRNA targeting the RBR gene transcript) or introducing an RNAi construct into the plant cell, wherein the expression of the small RNA (such as a miRNA or siRNA) can be controlled by a regulatory element, e.g. a constitutive or tissue-specific promoter.

Alternatively or in addition, transient decreased RBR expression can be accomplished by transient expression of an RBR repressor. A non-limiting example of an RBR repressor is a small noncoding RNA molecule, e.g. a miRNA or siRNA, targeting the RBR RNA transcript. The mature siRNA or miRNA can comprise at least 20, 21, 22, 23, 24 or at least 25 contiguous nucleotides. The mature siRNA or miRNA can comprise at least 20, 21, 22, 23, 24 or at least 25 contiguous nucleotides that that have at least about 95%, 96%, 97%, 98%, 99% or 100% sequence complementarity with a contiguous sequence in the endogenous RBR transcript.

The endogenous RBR transcript is preferably the endogenous RBR mRNA molecule, and preferably includes the 3′ and 5′ untranslated RBR sequence. Hence, the sequence of the noncoding small RNA can be, partly or completely, complementary to a sequence comprised in the RBR coding sequence or complementary to a sequence comprised in the 3′ or 5′ untranslated region of the RBR transcript. Preferably, the siRNA or miRNA can be partly or completely complementary to a sequence comprised in the RBR coding sequence. For example, at least 20, 21, 22, 23, 24 or at least 25 contiguous nucleotides of the small RNA molecule has at least about 80%, 85%, 90%, 95%, 96%, 97%, 98%, 99% or 100% sequence complementarity with a contiguous sequence of at least 20, 21, 22, 23, 24 or at least 25 contiguous nucleotides of the endogenous RBR transcript, respectively.

The skilled person understands how to design a small RNA molecule that is capable of downregulating endogenous RBR protein expression using conventional RNAi, wherein the RBR protein is a RBR protein as defined herein above.

In one embodiment, the small RNA molecule for inhibiting RBR expression can comprise a sequence having at least about 85%, 90%, 95%, 96%, 97%, 98%, 99% or 100% sequence identity with SEQ ID NO: 23. The small RNA molecule can comprise a sequence having at least about 85%, 90%, 95%, 96%, 97%, 98%, 99% or 100% sequence identity with SEQ ID NO: 24.

Transient activation of expression of the RBR repressor as defined herein can be achieved by placing the sequence encoding the RBR repressor upstream of, and operably linked to, an inducible promoter. The inducible promoter controlling the expression of the RBR repressor can be the same type of inducible promoter controlling the expression of at least one of the WOX5, WIND1, SHR, SCR, PLT1, PLT2, PLT3, PLT4, PLT5 or the PLT7 protein. Alternatively, the inducible promoter can be a different type of inducible promoter. Transient expression of the RBR repressor results in transient decreased expression of the RBR protein as defined herein.

Preferred examples of inducible promoters are described herein below in the second aspect.

Use of the Construct of the Invention

In one embodiment, the plant cell can be stably transformed with one or more nucleic acids comprising an expression cassette wherein the expression of at least one of WOX5, WIND1, SHR, SCR, PLT1, PLT2, PLT3, PLT4, PLT5 and PLT7, preferably at least one of WOX5, WIND1, SHR, SCR, PLT1, PLT4 or PLT5 is controlled by an inducible promoter. Optionally, the plant cell can be additionally stably transformed with a nucleic acid construct comprising an expression cassette wherein the expression of a RBR repressor is controlled by an inducible promoter. Several or all of the nucleic acids can be comprised in a single construct.

Preferably the plant cell is transformed with one or more constructs as described below in the second aspect.

In an embodiment, the invention thus pertains to a method for regenerating a plant cell, preferably regenerating a shoot from a plant cell, wherein the method comprises a step of
a) introducing, preferably stably introducing, in a plant cell one or more nucleic acids or nucleic acid constructs as defined herein in the second aspect;
b) maintaining the plant cell in a medium comprising an inducer;
c) optionally, detecting the expression levels of at least one or more proteins as defined herein, and optionally selecting a plant cell having an altered expression of at least one or more proteins as defined herein;
d) optionally maintaining the selected plant cell in a medium comprising the inducer during a period of time as defined herein; and
e) allowing the plant cell to regenerate, preferably into the shoot.

After the plant cell is regenerated, the inducer may be removed from the medium.

The construct can be introduced in the plant cell using any conventional means known in the art. Non-limiting examples of transformation methods include Agrobacterium transformation of plant tissue, microprojectile bombardment and electroporation. A preferred transformation method is Agrobacterium transformation, e.g. using the floral dip method (Clough et al, 1998).

Preferred concentrations of the inducer in step b) are concentrations that are known in the art to effectively activate the transactivator as defined herein, resulting in the subsequent activation of the inducible promoter. A preferable concentration is about 0.05 μM-50 μM, preferably about 0.1 μM-15 μM, preferably about 10 μM.

In one embodiment, the invention pertains to a method for regenerating a plant cell as defined herein, wherein the plant cell is not exposed to a plant growth hormone before, after and/or during the regeneration of the plant cell. The plant growth hormone can be at least one of a cytokinin or an auxin. Preferably, the plant cell is not exposed to concentrations of a plant growth hormone that would induce regeneration of the unmodified, e.g. wild-type, plant cell.

It is further contemplated herein that the plant cell can be exposed to a plant growth hormone, however the presence of the plant hormone is not an essential requirement for regeneration of the plant cell.

In a preferred embodiment, the invention pertains to a method for hormone-independent shoot regeneration from a plant cell, comprising the steps of
a) introducing or increasing in the plant cell the expression of a combination of proteins comprising at least:
   i) a WUSCHEL related homeobox 5 (WOX5) protein; and
   ii) a PLETHORA (PLT) protein selected from the group consisting of PLT1, PLT2, PLT3, PLT4, PLT5 and PLT7, preferably PLT1;
wherein the expression of at least one of the proteins of the combination of proteins is transiently introduced or increased; and
b) allowing the plant cell to regenerate into the shoot.

“Hormone-independent shoot regeneration” is understood herein as shoot regeneration without a required exposure of the plant cell to plant growth hormones. Preferably, the shoot regeneration is shoot organogenesis.

In one embodiment, the plant tissue is not wounded to stimulate plant regeneration. Wounding is a well-known step in tissue-culture techniques and the skilled person knows how to wound a plant cell and to induce wound stress. It is contemplated herein that a plant tissue can be wounded, however the wounding is not an essential step for regeneration.

Multicellular Plant Tissues

In one embodiment the plant cell is part of a multicellular tissue. The plant multicellular tissue can comprise differentiated cells. Alternatively or in addition, a multicellular tissue can comprise undifferentiated cells. In an embodiment, all cells of the multicellular tissue have at a certain time point or period an increased or introduced expression of one or more of the proteins as defined herein. Preferably, at least about 5%, 10%, 15%, 20%, 25%, 30%, 35%, 40%, 45%, 50%, 55%, 60%, 65%, 70%, 75%, 80%, 85%, 90%, 95%, 97%, 98%, 99% or about 100% of the cells of the multicellular tissue have, at a certain time point or period, an increased or de novo expression of at least one of a WOX5, a WIND1, SHR, a SCR, a WOX5, a PLT1, a PLT2, a PLT3, a PLT4, a PLT5 and a PLT7 protein and optionally a decreased expression of a RBR protein as defined herein.

In one embodiment, at least about 5%, 10%, 15%, 20%, 25%, 30%, 35%, 40%, 45%, 50%, 55%, 60%, 65%, 70%, 75%, 80%, 85%, 90%, 95%, 97%, 98%, 99% or about 100% of the cells of the multicellular tissue have, at a certain time point or period, an altered expression of all proteins of a combination of proteins as defined herein.

All cells of the multicellular plant tissue may be transformed to have an increased or introduced expression, preferably transiently increased or introduced expression, of one or more proteins as defined herein. In addition, all cells of the multicellular tissue may be transformed to have a decreased expression of a RBR protein as defined herein. Preferably, at least about 5%, 10%, 15%, 20%, 25%, 30%, 35%, 40%, 45%, 50%, 55%, 60%, 65%, 70%, 75%, 80%, 85%, 90%, 95%, 97%, 98%, 99% or about 100% of the cells of the multicellular tissue are transformed to have an altered expression of all proteins of a combination of proteins as defined herein.

The multicellular tissue can be a callus tissue, a plant organ or an explant. In an embodiment, the plant cell having an increased or introduced expression of one or more of the proteins as defined herein can be part of a plant organ. The plant organ can be a vegetative organ or a reproductive organ. A vegetative organ can be derived from the shoot system or root system. The organ can be at least one of roots, stems and leaves. A reproductive plant organ can be selected from the group consisting of flower, seed, fruit, cone, sori, strobili and gametophores. Preferably, the plant organ is a root. A root is herein understood as the non-leaf, non-nodes bearing parts of the plant's body. A typical arrangement of the cells in a root is root hair, epidermis, epiblem, cortex, endodermis, pericycle and the vascular tissue. In one embodiment, at least the cells, or part of the cells, of at least one of the root hair, epidermis, epiblem, cortex, endodermis, pericycle and the vascular tissue have a de novo or increased expression of at least one of a WOX5, a WIND1, SHR, a SCR, a WOX5, a PLT1, a PLT2, a PLT3, a PLT4, PLT5, and a PLT7 protein as defined herein and optionally have a decreased expression of an endogenous RBR protein. Preferably, at least the cells, or part of the cells, of at least one of the root hair, epidermis, epiblem, cortex, endodermis, pericycle and the vascular tissue have an altered expression of all proteins of a combination of proteins as defined herein at a time period as defined herein above.

Alternatively or in addition, the plant cell having altered expression of one or more of the proteins as defined herein can be part of a seedling.

Alternatively or in addition, the plant cell having altered expression of one or more of the proteins as defined herein can be part of a callus. A callus is a group of undifferentiated cells, preferably derived from adult cells. Callus cells can be capable of undergoing embryogenesis and formation of an entirely new plant. A plant callus is considered a growing mass of unorganized plant parenchyma cells. Callus can be produced from a single differentiated cell, and callus cells can be totipotent, being able to regenerate the whole plant body. The plant callus can be derived from a somatic tissue or tissues, e.g. a tissue that is available for explant culture. The cells that give rise to callus and somatic embryos preferably undergo rapid division and/or are partially undifferentiated such as meristematic tissue. The callus cell used in the method of the invention can be friable or compact. In addition or alternatively the callus cell can be rooty, shooty, or embryogenic callus (Ikeuchi M, Plant Cell. 2013 September; 25(9): 3159-3173).

In an embodiment, the plant cell having an altered expression of one or more of the proteins as defined herein can be part of an explant. An explant can be defined herein as a sample obtained from a part of a plant. The plant sample can be placed on a solid culture medium or liquid medium. Explants can be taken from many different parts of a plant, including portions of shoots, leaves, stems, flowers, roots, single undifferentiated cells and from mature cells. The cells preferably contain living cytoplasm and nuclei and are able to de-differentiate and resume cell division. An explant can be, or can be obtainable or obtained from, a meristematic end of a plant, such as e.g. the stem tip, axillary bud tip or root tip. In one embodiment, the explant is selected from the group consisting of a hypocotyl explant, a stem explant, a cotyledon explant, a root explant, a leaf explant, a flower explant and a meristematic tissue.

In a further embodiment, the plant cell is obtainable from a plant selected from the group consisting of Arabidopsis, barley, cabbage, canola, cassava, cauliflower, chicory, chrysanthemum, cotton, cucumber, eggplant, grape, hot pepper, lettuce, maize, melon, oilseed rape, potato, pumpkin, rice, rye, sorghum, soybean, squash, sugar cane, sugar beet, sunflower, sweet pepper, tomato, water melon, wheat, and zucchini. Optionally, the plant cell is obtainable from a plant of the family of Solanaceae, optionally of the genus Solanum, optionally the species Solanum lycopersicum or Solanum melongena. Optionally, the plant cell is obtainable from the family of Brassicaceae, optionally the species, or subspecies, is Raphanus sativus, Brassica oleracea, Brassica rapa, Brassica napus, Armoracia rusticana or Arabidopsis thaliana.

In an embodiment, the plant cell is a recalcitrant plant cell, i.e. a plant cell in which the regeneration efficiency fails or wherein the regeneration efficiency is poor. Non-limited examples include pepper, soybean, cucumber and sugar beet.

In a further embodiment, the plant cell is selected from the group consisting of Arabidopsis, tomato and sweet pepper.

Preferably the plant is not, or is not obtainable from, the genus Nicotiana. Preferably the plant is not, or not obtainable from, the species Nicotiana tabacum.

In an embodiment, the method comprises a step of forming a plant or plant part from the regenerated shoot. “Forming”, “producing” or “regenerating” a plant or plant part preferably includes a step of elongation of the formed shoot. Preferably, the elongated shoot is taken of the callus or explant.

Subsequent formation of roots may be induced in a separate root induction step, e.g. by incubating the shoots on a different culture medium (Thorpe, supra). Alternatively, the roots may be formed naturally with any further induction. The regenerant may subsequently be grown on soil e.g. to bear seeds.

The formed plant, plant part or plant product may comprise cells that have been transformed to have an altered, preferably a transiently altered, expression of all proteins or a combination of proteins as defined herein. The formed plant, plant part or plant product may consist of cells that have been transformed to have an altered, preferably a transiently altered, expression of all proteins or a combination of proteins as defined herein.

Preferably, at least about 0.0001%, 0.001%, 0.01%, 0.1%, 1%, 10%, 20%, 30%, 40%, 50%, 60%, 70%, 80%, 90%, 95%, 97%, 98%, 99% or about 100% of the cells of the formed plant, plant part or plant product have been transformed to have an altered, preferably a transiently altered, expression of all proteins of a combination of proteins as defined herein.

Preferably, the formed plant or plant part does not have an introduced or increased expression of at least one of a WIND1, a WOX5, a SHR, a SCR, a PLT1, a PLT2, a PLT3, a PLT4, a PLT5 or a PLT7 protein as defined herein. Preferably, the formed plant or plant part does not have a decreased expression of an RBR protein as defined herein. Hence, at least one of a WIND1, WOX5, a SHR, a SCR, WOX5, a PLT1, a PLT2, a PLT3, a PLT4, a PLT5, a PLT7 and a RBR protein as defined herein have endogenous expression levels in the formed plant or plant part. Preferably, the WIND1, WOX5, SHR, SCR, PLT1, PLT2, PLT3, PLT4, PLT5, PLT7 and a RBR protein as defined herein have endogenous expression levels in the formed plant or plant part. Preferably, the expression levels of all proteins of the combination of proteins as defined herein have endogenous expression levels in the formed plant or plant part. Nevertheless, the formed plant or plant part may comprise a construct as defined in the second aspect herein, which may be present in at least a detectable amount.

Endogenous protein expression levels are herein understood as unmodified, naturally occurring, protein expression levels. Hence, endogenous expression levels are the expression levels in an, e.g. otherwise identical, plant cell that is not modified to have an altered, e.g. introduced, increased or decreased, expression of the protein, or proteins, as defined herein. In the formed plant or plant part, the expression levels of the protein or proteins as defined herein can be the same or similar to the endogenous protein expression levels. Alternatively, the formed plant or plant part can maintain an elevated expression of one or more of the proteins as defined herein.

Nucleic Acid Construct

In a second aspect, the invention pertains to a nucleic acid molecule comprising at least one expression cassette, wherein the expression cassette comprises a nucleotide sequence encoding at least one of a WIND1 protein, a WOX5 protein, a SHR protein, a SCR protein, a PLT1 protein, a PLT2 protein, a PLT3 protein, a PLT4 protein, a PLT5 protein, a PLT7 protein and a RBR repressor as defined herein in the first aspect.

The terms “nucleic acid” and “nucleic acid molecule” can be used interchangeably herein.

In an embodiment, the nucleotide sequence encoding at least one of a WIND1, WOX5, SHR, SCR, PLT1, PLT2, PLT3, PLT4 PLT5 and PLT7 protein is a sequence as defined in the first aspect. In an embodiment, the nucleotide sequence encoding the RBR repressor has a sequence as defined in the first aspect.

Preferably, the nucleotide sequence encoding the SHR protein is has at least about 50%, 55%, 60%, 65%, 70%, 75%, 80%, 85%, 90%, 95%, 98%, 99% or 100% sequence identity with SEQ ID NO: 9. Preferably, the nucleotide sequence encoding the SCR protein is has at least about 50%, 55%, 60%, 65%, 70%, 75%, 80%, 85%, 90%, 95%, 98%, 99% or 100% sequence identity with SEQ ID NO: 10. Preferably, the nucleotide sequence encoding the WOX5 protein has at least about 50%, 55%, 60%, 65%, 70%, 75%, 80%, 85%, 90%, 95%, 98%, 99% or 100% sequence identity with SEQ ID NO: 11. Preferably, the nucleotide sequence encoding the PLT1 protein has at least about 50%, 55%, 60%, 65%, 70%, 75%, 80%, 85%, 90%, 95%, 98%, 99% or 100% sequence identity with SEQ ID NO: 12. Preferably, the nucleotide sequence encoding the PLT2 protein has at least about 50%, 55%, 60%, 65%, 70%, 75%, 80%, 85%, 90%, 95%, 98%, 99% or 100% sequence identity with SEQ ID NO: 13. Preferably, the nucleotide sequence encoding the PLT3 protein has at least about 50%, 55%, 60%, 65%, 70%, 75%, 80%, 85%, 90%, 95%, 98%, 99% or 100% sequence identity with SEQ ID NO: 14. Preferably, the nucleotide sequence encoding the PLT4 protein has at least about 50%, 55%, 60%, 65%, 70%, 75%, 80%, 85%, 90%, 95%, 98%, 99% or 100% sequence identity with SEQ ID NO: 15. Preferably, the nucleotide sequence encoding the PLT5 protein has at least about 50%, 55%, 60%, 65%, 70%, 75%, 80%, 85%, 90%, 95%, 98%, 99% or 100% sequence identity with SEQ ID NO: 16. Preferably, the nucleotide sequence encoding the PLT7 protein has at least about 50%, 55%, 60%, 65%, 70%, 75%, 80%, 85%, 90%, 95%, 98%, 99% or 100% sequence identity with SEQ ID NO: 19. Preferably, the nucleotide sequence encoding the WIND1 protein has at least about 50%, 55%, 60%, 65%, 70%, 75%, 80%, 85%, 90%, 95%, 98%, 99% or 100% sequence identity with SEQ ID NO: 29. Preferably, the nucleotide sequence encoding the RBR repressor has at least about 85%, 90%, 95%, 98%, 99% or 100% sequence identity with SEQ ID NO: 23 or preferably, the nucleotide sequence encoding the RBR repressor has at least about 85%, 90%, 95%, 98%, 99% or 100% sequence identity with SEQ ID NO: 24.

In one embodiment, one promoter can control the expression of two or more proteins as defined herein. In such case, the sequences encoding the two or more proteins are preferably separated with e.g. an internal ribosome entry site (IRES) or other suitable element that allows for translation initiation in a cap-independent manner.

In an embodiment, each one of WIND1, WOX5, SHR, SCR, PLT1, PLT2, PLT3, PLT4, PLT5, PLT7 and RBR repressor is independently controlled by a promoter. Hence, the expression cassette can comprise a promoter controlling the expression of a SHR protein as defined herein, or the expression cassette can comprise a promoter controlling the expression of a SCR protein as defined herein, or the expression cassette can comprise a promoter controlling the expression of a WOX5 protein as defined herein, or the expression cassette can comprise a promoter controlling the expression of a PLT1 protein as defined herein, or the expression cassette can comprise a promoter controlling the expression of a PLT2 protein as defined herein, or the expression cassette can comprise a promoter controlling the expression of a PLT3 protein as defined herein, or the expression cassette can comprise a promoter controlling the expression of a PLT4 protein as defined herein or the expression cassette can comprise a promoter controlling the expression of a PLT5 protein as defined herein, or the expression cassette can comprise a promoter controlling the expression of a PLT7 protein as defined herein, or the expression cassette can comprise a promoter controlling the expression of a WIND1 protein as defined herein or the expression cassette can comprise a promoter controlling the expression of RBR repressor as defined herein.

The promoter in the expression cassette is preferably a constitutive promoter, a tissue-specific promoter or an inducible promoter. Preferably, the nucleotide sequence encoding a protein or repressor as defined herein is operably linked to an inducible promoter, preferably an inducible promoter as defined herein below.

Inducible promoters are well-known in the art. A preferred inducible promoter can be switched on by an inducing agent and is typically active as long as it is exposed to the inducing agent (i.e. inducer). The inducing agent can be a chemical agent, such as a metabolite, growth regulator, herbicide, or phenolic compound, or a physiological stress directly imposed upon the plant cell such as cold, heat, salt, toxins, or through the action of a microbial pathogen or an insecticidal pest.

Accordingly, inducible promoters can be utilized to regulate expression of the protein or proteins as defined in the first aspect.

The inducible promoter in the expression cassette of the current invention can be a stress-inducible promoter, a light-inducible promoter or a chemical-inducible promoter.

Examples of abiotic stress-inducible promoters include, but not limited to, salt-inducible promoters such as RD29A (Yamaguchi-Shinozalei et al., 1993); drought-inducible promoters such as maize rabl7 gene promoter (Pla et. al., 1993), maize rab28 gene promoter (Busk et. al., 1997) and maize Ivr2 gene promoter (Pelleschi et. al., 1999); heat-inducible promoters such as heat tomato hsp80-promoter from tomato (U.S. Pat. No. 5,187,267) and the PHS1 heat shock protein gene (Takahashi et al., 1989).

Examples of light-inducible promoters include the three chlorophyll a/b light harvesting protein promoters (Leutwiler et al., 1986) and the pre-ferredoxin promoter (Vorst et al., 1990).

Other examples of inducible promoters include the promoter from the 27 kD subunit of the glutathione-S-transferase, isoform II (GST-II-27). This promoter is induced by chemical compounds known as “herbicide safeners”, which can be applied onto the plant cell to induce the promoter. See PCT/GB92/01187 and PCT/GB90/00101, incorporated herein by reference. This promoter functions in both monocotyledons and dicotyledons. Similarly, the alcA/alcR gene activation system of Aspergillus nidulans (e.g. comprising the AlcA element of SEQ ID NO: 32, or any sequence having at least about 50%, 55%, 60%, 65%, 70%, 75%, 80%, 85%, 90%, 95%, 98%, 99% or 100% sequence identity with SEQ ID NO: 32, and the AlcR transactivator sequence of SEQ ID NO: 31, or any sequence having at least about 50%, 55%, 60%, 65%, 70%, 75%, 80%, 85%, 90%, 95%, 98%, 99% or 100% sequence identity with SEQ ID NO: 31) can be used for chemically-inducible gene expression.

The AlcR/AlcA gene activation system is ethanol inducible. Other suitable systems for chemical induction include the a/c-switch, GVE/VGE, GVG, pOp6/LhGR (Craft et al., 2005), and XVE systems (Moore et al, 2006, in particular FIG. 3 and Table 5, which are incorporated herein by reference).

The LhGR and GVG systems use dexamethasone as an inducer. The XVE system uses 17-β-oestradiol as an inducer and the VGE system uses Methoxyfenozide (Intrepid-F2). The skilled person knows how to use these inducible systems for the cloning the suitable promoter elements upstream of the nucleotide sequence encoding a protein as defined herein in the first aspect in order to achieve an inducible expression system.

In an embodiment, the inducible system used for the transient activation of expression as defined herein is at least one of the LhGR, GVG or XVE system or a combination thereof.

The nucleic acid molecule, or nucleic acid molecules as defined herein can be part of a nucleic acid construct.

The invention further pertains to a nucleic acid construct comprising two or more expression cassettes, such as 2, 3, 4, 5, 6, 7 or 8 different expression cassettes as defined herein. Alternatively or in addition, each expression cassette as defined herein can be present more than once, e.g. 2, 3, 4, or 5 times, within the nucleic acid construct. Preferably, each expression cassette comprises a sequence encoding a WIND1, WOX5, SHR, SCR, PLT1, PLT2, PLT3, PLT4, PLT5 or PLT7 protein or RBR repressor as defined herein under the control of an inducible promoter.

In an embodiment, the nucleic acid construct can comprise at least
one first nucleic acid sequence comprising an expression cassette having a sequence encoding a WIND1, WOX5, SHR, SCR, WOX, PLT1, PLT2, PLT3, PLT4, PLT5 or PLT7 protein or a RBR repressor as defined herein under the control of an inducible promoter; and
a second nucleic acid sequence comprising a second expression cassette having a sequence encoding a transactivator operably linked to a regulatory element. The transactivator can be a transactivator as defined herein below. Preferably the regulatory element is a strong constitutive promoter such as CaMV, G10-90, CsV, TCTP2 or a UBQ10 promoter. Upon binding an inducer, the expressed transactivator can bind to the inducible promoter of the first nucleic acid molecule and initiate transcription.

The nucleic acid molecule or construct of the invention may comprise expression cassettes for the expression of each of the combinations or proteins and/or repressor as defined in the first aspect herein. Optionally, the combinations of proteins and/or repressor of the first aspect are on separate constructs, e.g. 1, 2, 3, 4, 5, 6, 7, 8, 9, 10, 11 or 12 different constructs, however, preferably the combinations are present on a single construct.

The GVG System

The nucleic acid molecule, and the expression cassette comprised within the nucleic acid molecule, can comprise one or more UAS elements, which elements are preferably linked to a minimal promoter, such as the −46 35S minimal promoter. The minimal promoter and the elements are located preferably upstream of, and operably linked to, the sequence encoding a protein or repressor as defined in the first aspect. Preferably the nucleic acid molecule, and the expression cassette comprised within the nucleic acid molecule, can comprise at least about four or five UAS elements located upstream of the sequence encoding a protein or repressor as defined in the first aspect. The UAS sequence has preferably at least about 80%, 85%, 90%, 95%, 96%, 97%, 98%, 99% or 100% sequence identity with SEQ ID NO: 22. The UAS elements are preferably operably linked to the sequence encoding a protein or repressor as defined in the first aspect. These UAS element or elements can be bound by a transactivator, resulting in transcription of a protein or repressor as defined herein.

The transactivator binding the UAS element or elements preferably comprises a GAL4 DNA binding domain, a VP16 domain and a glucocorticoid receptor (GR) domain. Such GVG systems are well-known in the art and e.g. described in Moore et al (2006). The transactivator can initiate transcription upon binding dexamethasone, or a derivative thereof. Hence in an embodiment of the invention, a nucleic acid can comprise an expression cassette, wherein the expression cassette comprises a sequence encoding a transactivator. Preferably, the transactivator is a protein comprising a domain binding to the UAS elements, preferably a GAL4 domain. Preferably, the transactivator further comprises a GR domain and a VP16 domain. In a preferred embodiment, the nucleotide sequence encoding the transactivator has at least about 50%, 55%, 60%, 65%, 70%, 75%, 80%, 85%, 90%, 95%, 98%, 99% or 100% sequence identity with SEQ ID NO: 20.

The LhGR System

In an embodiment the nucleic acid molecule, and the expression cassette comprised within the nucleic acid molecule, can comprise one or more LacOp elements, which elements are preferably linked to a minimal promoter, such as the −46 35S minimal promoter. The minimal promoter and the elements are located preferably upstream of, and operably linked to, the sequence encoding a protein or repressor as defined in the first aspect. Preferably the nucleic acid molecule, and the expression cassette comprised within the nucleic acid molecule, can comprise at least about five or six LacOp elements located upstream of the sequence encoding a protein or repressor as defined in the first aspect. The LacOp sequence preferably has at least about 80%, 85%, 90%, 95%, 96%, 97%, 98%, 99% or 100% sequence identity with SEQ ID NO: 25. The LacOp elements are preferably operably linked to the sequence encoding a protein or repressor as defined in the first aspect. These LacOp element or elements can be bound by a transactivator, resulting in transcription of a protein or repressor as defined herein.

The transactivator binding the LacOp element or elements preferably comprises a GAL4 DNA binding domain, a VP16 domain and a glucocorticoid receptor (GR) domain. Such LacOp systems are well-known in the art and e.g. described in Moore et al (2006). The transactivator can initiate transcription upon binding dexamethasone, or a derivative thereof. Hence in an embodiment of the invention, a nucleic acid can comprise an expression cassette, wherein the expression cassette comprises a sequence encoding the transactivator. Preferably, the transactivator is a protein comprising a domain binding to the LacOp elements, preferably a GAL4 domain. Preferably, the transactivator further comprises a GR domain and a VP16 domain. In a preferred embodiment, the nucleotide sequence encoding the transactivator has at least about 50%, 55%, 60%, 65%, 70%, 75%, 80%, 85%, 90%, 95%, 98%, 99% or 100% sequence identity with SEQ ID NO: 21. Preferably, the transactivator has an amino acid sequence of at least about 50%, 55%, 60%, 65%, 70%, 75%, 80%, 85%, 90%, 95%, 98%, 99 or 100% sequence identity with SEQ ID NO: 58.

The XVE System

The nucleic acid molecule, and the expression cassette comprised within the nucleic acid molecule, can comprise one or more LexAop elements, which elements are preferably linked to a minimal promoter, such as the −46 35S minimal promoter. The minimal promoter and the elements are located preferably upstream of, and operably linked to, the sequence encoding a protein or repressor as defined in the first aspect. Preferably the nucleic acid molecule, and the expression cassette comprised within the nucleic acid molecule, can comprise at least about seven or eight LexAop elements located upstream of the sequence encoding a protein or repressor as defined in the first aspect. The LexAop sequence has preferably at least about 80%, 85%, 90%, 95%, 96%, 97%, 98%, 99% or 100% sequence identity with SEQ ID NO: 26. The LexAop elements are preferably operably linked to the sequence encoding a protein or repressor as defined in the first aspect. These LexAop element or elements can be bound by a transactivator, resulting in transcription of a protein or repressor as defined herein.

The transactivator binding the LexAop element or elements preferably comprises a LEXA DNA binding domain, a VP16 domain and an oestrogen receptor (ER) domain. Such XVE systems are well-known in the art and e.g. described in Moore et al (2006). The transactivator can initiate transcription upon binding β-estradiol, or a derivative thereof. Hence in an embodiment of the invention, a nucleic acid can comprise an expression cassette, wherein the expression cassette comprises a sequence encoding a transactivator. Preferably, the transactivator is a protein comprising a domain binding to the LexAop elements, preferably a LEXA domain. Preferably, the transactivator further comprises a ER domain and a VP16 domain. In a preferred embodiment, the sequence encoding the transactivator has at least about 50%, 55%, 60%, 65%, 70%, 75%, 80%, 85%, 90%, 95%, 98%, 99% or 100% sequence identity with SEQ ID NO: 27.

In one embodiment, the nucleic acid construct comprises at least one or more expression cassettes as defined herein. Preferably, the expression cassette comprises a sequence encoding a WIND1, WOX5, SHR, SCR, PLT1, PLT2, PLT3, PLT4, PLT5 or PLT7 protein or a RBR repressor as defined herein, wherein the sequence is operably linked to an inducible promoter as defined herein. The construct can comprise more than one such expression cassette. In one embodiment, the construct can comprise the following elements:
a nucleic acid sequence comprising an expression cassette encoding a WIND1 protein as defined herein operably linked to an inducible promoter as defined herein; and
a nucleic acid sequence comprising an expression cassette encoding a WOX5 protein as defined herein operably linked to an inducible promoter as defined herein.

In a further embodiment, the construct can comprise the following elements:
a nucleic acid sequence comprising an expression cassette encoding a WIND1 protein as defined herein operably linked to an inducible promoter as defined herein;
a nucleic acid sequence comprising an expression cassette encoding a WOX5 protein as defined herein operably linked to an inducible promoter as defined herein; and
a nucleic acid sequence comprising an expression cassette encoding a PLT1 protein as defined herein operably linked to an inducible promoter as defined herein

In an embodiment, the construct can comprise the following elements:
a nucleic acid sequence comprising an expression cassette encoding a SHR protein as defined herein operably linked to an inducible promoter as defined herein;
a nucleic acid sequence comprising an expression cassette encoding a SCR protein as defined herein operably linked to an inducible promoter as defined herein;
a nucleic acid sequence comprising an expression cassette encoding a WOX5 protein as defined herein operably linked to an inducible promoter as defined herein;
a nucleic acid sequence comprising an expression cassette encoding a PLT1 protein as defined herein operably linked to an inducible promoter as defined herein;
a nucleic acid sequence comprising an expression cassette encoding a PLT4 protein as defined herein operably linked to an inducible promoter as defined herein; and
a nucleic acid sequence comprising an expression cassette encoding a PLT5 protein as defined herein operably linked to an inducible promoter as defined herein.

In a further embodiment, the construct can comprise the following elements:
a nucleic acid sequence comprising an expression cassette encoding a WIND1 protein as defined herein operably linked to an inducible promoter as defined herein;
a nucleic acid sequence comprising an expression cassette encoding a SHR protein as defined herein operably linked to an inducible promoter as defined herein;
a nucleic acid sequence comprising an expression cassette encoding a SCR protein as defined herein operably linked to an inducible promoter as defined herein;
a nucleic acid sequence comprising an expression cassette encoding a WOX5 protein as defined herein operably linked to an inducible promoter as defined herein;
a nucleic acid sequence comprising an expression cassette encoding a PLT1 protein as defined herein operably linked to an inducible promoter as defined herein;
a nucleic acid sequence comprising an expression cassette encoding a PLT4 protein as defined herein operably linked to an inducible promoter as defined herein;
a nucleic acid sequence comprising an expression cassette encoding a PLT5 protein as defined herein operably linked to an inducible promoter as defined herein; and
a nucleic acid sequence comprising an expression cassette encoding a RBR repressor as defined herein operably linked to an inducible promoter as defined herein.

In an embodiment, the expression of SHR, SCR, the PLT proteins and WOX5 as defined herein is controlled by the GVG system as defined herein above.

In an embodiment, the expression of the WIND1 protein and the RBR repressor as defined herein is controlled by the XVE system as defined herein above.

In an embodiment the construct further comprises at least one expression cassette for expression of a transactivator. Preferably, the construct further comprises two expression cassettes for the expression of two transactivators, wherein the sequence encoding the transactivators preferably have at least about 50%, 55%, 60%, 65%, 70%, 75%, 80%, 85%, 90%, 95%, 98%, 99% or 100% sequence identity with SEQ ID NO: 20 or SEQ ID NO: 27, respectively.

In a further embodiment, the construct can comprise a resistance gene to select for plants that have stably integrated the construct, for example, but not limited to a Streptomyces hygroscopicus BASTA herbicide resistance marker.

Composition

In a third aspect, the invention pertains to a composition. The composition can comprise a nucleic acid construct as defined herein above in the second aspect. In an embodiment, the composition comprises e.g. a stabilizer, salt or diluent.

Alternatively or in addition, the composition can comprise two nucleic acid constructs, wherein the first nucleic acid construct comprises a first expression cassette having a sequence encoding a WIND1, a WOX5, a SHR, a SCR, a PLT1, a PLT2, a PLT3, a PLT4, a PLT5 or a PLT7 protein as defined herein or a RBR repressor as defined herein, under the control of an inducible promoter. The composition further comprises a second construct, wherein the second construct comprises an expression cassette having a sequence encoding a transactivator operably linked to a regulatory element. Preferably the regulatory element is a strong constitutive promoter such as a CaMV, a G10-90, a CsV, a TCTP2 or a UBQ10 promoter. Upon binding an inducer, the expressed transactivator can bind to the inducible promoter of the first construct and initiate transcription.

The first construct can comprise additional expression cassettes each having a sequence encoding a WIND1, WOX5, SHR, SCR, PLT1, PLT2, PLT3, PLT4, PLT5 or PLT7 protein or a RBR repressor as defined herein, under the control of an inducible promoter.

Alternatively or in addition, the composition can comprise additional constructs comprising an expression cassette having a sequence encoding a WIND1, WOX5, SHR, SCR, PLT1, PLT2, PLT3, PLT4, PLT5 or PLT7 protein or a RBR repressor as defined herein, under the control of an inducible promoter

Plant and Plant Parts

In a fourth aspect, the invention pertains to a plant cell comprising at least one of
i) the nucleic acid molecule as defined in the second aspect; and
ii) the nucleic acid construct as defined in the second aspect.

Preferably, the plant cell can have an introduced or an increased expression of at least one of a WIND1, WOX5, SHR, SCR, PLT1, PLT2, PLT3, PLT4, PLT5 and PLT7 protein and optionally a decreased expression of an endogenous RBR protein as defined herein upon exposure of the plant cell to an inducer as defined herein. Preferably at one time point or period as defined in the first aspect, the plant cell has an introduced or an increased expression of at least WOX5 and PLT1 upon exposure of the plant cell to an inducer as defined herein.

The plant cell is preferably obtainable from Arabidopsis, barley, cabbage, canola, cassava, cauliflower, chicory, chrysanthemum, cotton, cucumber, eggplant, grape, hot pepper, lettuce, maize, melon, oilseed rape, potato, pumpkin, rice, rye, sorghum, soybean, squash, sugar cane, sugar beet, sunflower, sweet pepper, tomato, water melon, wheat, and zucchini. Optionally, the plant cell is obtainable from a plant of the family of Solanaceae, optionally of the genus Solanum, optionally the species Solanum lycopersicum or Solanum melongena. Optionally, the plant cell is obtainable from the family of Brassicaceae, optionally the species, or subspecies, is Raphanus sativus, Brassica oleracea, Brassica rapa, Brassica napus, Armoracia rusticana or Arabidopsis thaliana.

In a fifth aspect, the invention pertains to a shoot, plant or plant part obtainable or obtained by the method of the invention as defined herein. Plant cells obtained from the plant or plant part preferably have endogenous expression levels of at least one of WOX5, SHR, SCR, WIND1, PLT1, PLT2, PLT3, PLT4, PLT5 and RBR. Preferably, the plant cells obtained from the plant or plant part have endogenous WOX5, SHR, SCR, WIND1, PLT1, PLT2, PLT3, PLT4, PLT5 and RBR protein levels.

Plant cells of the shoot, plant or plant part obtainable or obtained by the method of the invention as defined herein can comprise at least, or at least part of, one of:
i) a nucleic acid molecule in the second aspect; and
ii) the nucleic acid construct as defined in the second aspect.

The plant obtainable or obtained by the method of the invention is preferably selected from the group consisting of Arabidopsis, barley, cabbage, canola, cassava, cauliflower, chicory, chrysanthemum, cotton, cucumber, eggplant, grape, hot pepper, lettuce, maize, melon, oilseed rape, potato, pumpkin, rice, rye, sorghum, soybean, squash, sugar cane, sugar beet, sunflower, sweet pepper, tomato, water melon, wheat, and zucchini. Optionally, the plant obtainable or obtained by the method of the invention is from the family of Solanaceae, optionally of the genus Solanum, optionally the species Solanum lycopersicum or Solanum melongena. Optionally, the plant obtainable or obtained by the method of the invention is from the family of Brassicaceae, optionally the species, or subspecies, is Raphanus sativus, Brassica oleracea, Brassica rapa, Brassica napus, Armoracia rusticana or Arabidopsis thaliana.

Alternatively about 5%, 10%, 15%, 20%, 25%, 30%, 35%, 40%, 45%, 50%, 55%, 60%, 65%, 70%, 75%, 80%, 85%, 90%, 95%, 97%, 98% or about 99% of the plant cells have at least, or at least part of, one of:
i) a nucleic acid molecule as defined in the second aspect; and
ii) the nucleic acid construct as defined in the second aspect.

In one embodiment, the plant part is a seed, a fruit or a non-propagating material.

In a sixth aspect, the invention concerns a product derived from the plant or plant part obtainable or obtained by the method of the invention, e.g. fruits, leaves, plant organs, plant fats, plant oils, plant starch, and plant protein fractions, either crushed, milled or still intact, mixed with other materials, dried, frozen, and so on. These products may be non-propagating. Preferably, said plant product comprises at least or at least part of one of:
i) the nucleic acid molecule as defined in the second aspect; and
ii) the nucleic acid construct as defined in the second aspect.

Preferably, these products comprise at least fractions of said nucleic acid and/or construct, which allows to assess that the plant product is derived from a plant obtained by the method of the first aspect of the invention as defined herein.

In a seventh aspect, the invention concerns progeny of the plant cell, plantlet or plant obtainable or obtained by the method of the invention. The plant cells of the progeny can an increased or induced expression of at least one of a WOX5, SHR, SCR, WIND1, PLT1, PLT2, PLT3, PLT4 PLT5 and PLT7 protein as defined herein, and optionally a decreased expression of a RBR protein as defined herein, upon exposure to an inducer as defined herein.

Said progeny may comprise the nucleic acid and/or an expression construct as defined in the second aspect of the invention as defined herein. Preferably, the progeny can generate a shoot from a plant cell after exposure to one or more inducers as defined herein.

In an eighth aspect, the invention pertains to a method for producing a plant, wherein the method comprises the steps of regenerating a shoot from a plant cell as defined in the first aspect above and wherein the shoot is developed into a plant.

Use of the Proteins of the Invention for Regeneration

In a ninth aspect, the invention related to the use of a combination of proteins and/or repressor as defined herein for regenerating a shoot from a plant cell. Preferably, the invention concerns the use of a combination of at least one of:
a WOX5 protein and a PLT1 protein;
a WIND1 protein, a WOX5 protein and a PLT1 protein;
a SHR protein, a SCR protein, a WOX5 protein, a PLT1, PLT4 and PLT5 protein; and
a WIND1 protein, and a SHR protein, a SCR protein, a WOX5 protein, a PLT1, PLT4 and PLT5 protein and a RBR repressor
   for regenerating a shoot from a plant cell. The plant cell is preferably a plant cell as defined herein above.

In a tenth aspect, the invention concerns the use of a nucleic acid molecule or a nucleic acid construct as defined in the second aspect for regenerating a shoot from a plant cell. The plant cell is preferably a plant cell as defined herein above.

### EXAMPLES

### Example 1

Compositions and methods are provided for the induction of shoot regeneration from plant cells using a set of defined Arabidopsis (transcription) factors. Compositions include constructs with following factors: WIND1 (Iwase et al., 2011), an artificial microRNA targeting RETINOBLASTMA RELATED (amiRBR) (Cruz-Ramirez et al., 2013), SHORT ROOT (SHR) (Benfey et al., 1993), SCARECROW (SCR) (Di Laurenzio et al., 1996), PLETHORA1 (PLT1) (Aida et al., 2004), BABYBOOM/PLETHORA4 (PLT4) (Boutilier et al., 2002, Galinha et al., 2007), PLETHORA5 (PLT5) (Tsuwamoto et al., 2010, Prasad et al., 2011), and WUSCHEL RELATED HOMEOBOX 5 (WOX5) (Sarkar et al., 2007). The chosen factors were divided into two different sets and placed under control of different constitutively expressed chemically inducible transactivation systems to allow timed and regulated expression. Constructs were built from modular parts using the golden gate cloning method (Engler et al., 2014). Transactivation systems include GVG/UAS, consisting of a chimeric transcription, GVG, assembled by fusion of the DNA-binding domain of the yeast transcription factor GAL4 (G), the transactivating domain of the herpes viral protein VP16 (V), and the receptor domain of the rat glucocorticoid receptor (G) that binds to a promoter containing six copies of the GAL4 upstream activating sequence fused to the −46 35S minimal promoter (UAS) (Aoyama and Chua, 1997) and XVE/lexA, consisting of a chimeric transcription activator, XVE, was assembled by fusion of the DNA-binding domain of the bacterial repressor LexA (X), the transactivating domain of the herpes viral protein VP16 (V) and the regulatory region of the human estrogen receptor (E) that binds to a promoter containing eight copies of the LexA operator sequence fused to the −46 35S minimal promoter (lexA) (Zuo et al., 2000).

As further detailed herein below, methods are provided for the timed induction of factors that involve the application of chemical inducers to separately or simultaneously induce controlled transcription. Methods using these constructs to induce shoot regeneration from plant cells independent of externally added plant hormones are also provided.

Material and Methods

Construct Generation

A vector was constructed comprising the following transcriptional elements:
the Streptomyces hygroscopicus BASTA herbicide resistance marker (BAR; Thompon et al., 1987), operably linked to the Agrobacterium tumefaciens nopaline synthase promoter (NOSp; SEQ ID NO: 33) and the A. tumefaciens nopaline synthase terminator (NOST; SEQ ID NO: 34);
the chimeric transcription factor XVE (Zuo, 2000; SEQ ID NO: 27) coding sequence operably linked to the A. thaliana TRANSLATIONALLY-CONTROLLED TUMOR PROTEIN 1 promoter (AtTCTP1) (Czechowski et al., 2005; SEQ ID NO: 35) and the A. thaliana UBIQUITIN10 terminator (AtUBQ10Ter; SEQ ID NO: 36);
the chimeric transcription factor GVG (Aoyama, 1997; SEQ ID NO: 20) coding sequence, operably linked to the G10:90 synthetic promoter (Ishige, 1999; SEQ ID NO: 37) and the Pisum sativum ribulose-1,5-bisphosphate carboxylase small subunit terminator (rbcST; SEQ ID NO: 38) and NOST (SEQ ID NO: 34);
the artificial microRNA for Gene-silencing Overcome specific for A. thaliana Retinoblastoma-related (AmiRBR) coding sequence (Cruz-Ramirez, 2013; SEQ ID NO: 18), operably linked to the Escherichia coli LexA (Zuo, 2000) promoter (SEQ ID NO: 39), the Cauliflower mosaic virus 35S minimal promoter (35Smini) (Odell et al., 1985; SEQ ID NO: 40) and the Solanum lycopersicum ATPase (SIATPase) terminator (Engler, 2014; SEQ ID NO: 41);
the A. thaliana WOUND INDUCED DEDIFFERENTIATION1 coding sequence (AtWIND1; SEQ ID NO: 29) comprising the 4×Myc C-tag (4×Myc; SEQ ID NO: 42), operably linked to the LexA+35Smini promoter (LexAop; SEQ ID NO: 26) and the A. thaliana UBIQUITIN3 terminator (AtUBQ3; SEQ ID NO: 44);
the A. thaliana SHORT-ROOT coding sequence (AtSHR; SEQ ID NO: 9) comprising the 3×FLAG octapeptide C-tag (3×FLAG; SEQ ID NO: 45), operably linked to the Saccharomyces cerevisiae Upstream Activation Sequence promoter (UASp; SEQ ID NO: 47), the 35Smini (SEQ ID NO: 40) and the A. tumefaciens Purified octopine synthase terminator (AtuOCS; SEQ ID NO: 48);
the A. thaliana SCARECROW coding sequence (AtSCR; SEQ ID NO: 10) comprising the simian virus 5 C-tag (V5; SEQ ID NO: 49), operably linked to the UAS+35Smini promoter and the A. tumefaciens mannopine synthase terminator (AtuMas; SEQ ID NO: 51);
the A. thaliana PLETHORA 1 coding sequence (AtPLT1) comprising the bacteriophage T7 gene 10 C-tag (T7; SEQ ID NO: 52), operably linked to the UAS+35Smini promoter (UAS; SEQ ID NO: 22) and the A. thaliana heat shock protein 18.2 terminator (AtHSP; SEQ ID NO: 54);
the A. thaliana BABYBOOM coding sequence (AtPLT4; SEQ ID NO: 15) comprising the T7 C-tag (SEQ ID NO: 52), operably linked to the UAS+35Smini promoter (UAS; SEQ ID NO: 22) and the A. thaliana UBIQUITIN5 terminator (AtUBQ5; SEQ ID NO: 55);
the A. thaliana PLETHORA 5 coding sequence (AtPLT5; SEQ ID NO: 16) comprising the 3×FLAG C-tag (3×FLAG; SEQ ID NO: 45), operably linked to the UAS+35Smini promoter (UAS; SEQ ID NO: 22) and the A. thaliana alcohol dehydrogenase terminator (AtADH; SEQ ID NO: 56); and,
the A. thaliana WUSCHEL RELATED HOMEOBOX 5 coding sequence (AtWOX5; SEQ ID NO: 11) comprising the V5 C-tag (SEQ ID NO: 49), operably linked to the UAS+35Smini promoter (UAS; SEQ ID NO: 22) and the Cauliflower mosaic virus 35S terminator (T35S; SEQ ID NO: 57).

Arabidopsis Transformation:

For plant transformation, vector SHOOT REGENERATION was introduced into A. tumefaciens (strain C58C1.pMP90) by electroporation. Transformant A. tumefaciens were used to transform A. thaliana plants of the Col-0 ecotype by the floral dip method (Clough, 1998).

Germination:

Seeds were surface sterilised by chlorin gas by the vapor sterilisation method (Lindsey, 2017). After sterilisation, the seeds were suspended in a 0.1% agarose solution, before being stratified at 4° C. in darkness for 48 hours. Subsequently, the seeds were plated on germination medium under sterile conditions. The seeds were plated on a fine nylon mesh (100 μm), which allowed for the contact with the medium without allowing seedlings to penetrate the mesh. The plates with the seedlings were sealed with surgical tape (3M, Micropore) to prevent desiccation. The plates were placed in upright position in a growth chamber (22° C., 120-150 μmol/m²s with 16 h light/8 h dark photoperiodlight). The plates were left to grow in these circumstances for 5 days.

Induction:

After 5 days of growth, the seedlings were transplanted under sterile conditions by transporting the mesh on which they were growing from the germination medium to the induction medium. The plates with the seedlings were sealed with surgical tape (3M, Micropore) to prevent desiccation. The plates were placed back in the growth chamber in upright position. The plates were left to grow in this condition for 14 days. Shoots started appearing in the second week of induction.

Media Used:

Germination Medium

5 g Sucrose (Duchefa, product number S0809.5000)

1.1 g MS+vitamins (Duchefa, product number M0222.0050)

4 g Plant Agar (Duchefa, product number P1001.1000)

0.5 g/L MES 5.8 mg/L

Induction Medium

5 g Sucrose (Duchefa, product number S0809.5000)

1.1 g MS+vitamins (Duchefa, product number M0222.0050)

4 g Plant Agar (Duchefa, product number P1001.1000)

0.5 g/L MES 5.8 mg/L

400 μL 10 mM Dexamethasone (Sigma Aldrich product number 101152255) dissolved in DMSO (Sigma Aldrich product number 100897077)

Results

Plants transformed with the SHOOT REGENERATION vector (XVE-transactivated AmiRBR and AtWIND1; GVG-transactivated AtSHR, AtSCR, AtPLT1, AtPLT4, AtPLT5 and AtWOX5) were grown on medium containing 10 μM 17β-estradiol (EST: activation of XVE) and/or 10 μM dexamethasone (DEX: activation of GVG).

In transformants of 11 lines containing the SHOOT REGENERATION vector that were induced with both EST and DEX, halted growth of the primary root was observed. Callus formation on the primary root was observed in transformants of every independent line. Formation of green calli was observed in transformants of 55% of transgenic lines, and transformants of 67% of these lines regenerated shoots from the observed green calli without the application of plant hormones (Table 3). This represents 36% of all transformants tested (Table 2).

In transformants of all lines containing the SHOOT REGENERATION vector that were induced with DEX alone, halted growth of the primary root was observed. Callus formation on the primary root was observed in transformants of 82% of transgenic lines. Formation of green calli was observed in transformants of 44% of these transgenic lines, and transformants of all of these lines (100%) regenerated shoots from the observed green calli without the application of plant hormones (Table 3). This represents 36% of all transformants tested (Table 2).

Plants transformed with the SHOOT REGENERATION-2 vector (XVE-transactivated AtWIND1; GVG-transactivated AtPLT1 and AtWOX5) were grown on medium containing 10 μM 17β-estradiol (EST: activation of XVE) and/or 10 μM dexamethasone (DEX: activation of GVG).

In transformants of all 25 lines containing the SHOOT REGENERATION-2 vector that were induced with both EST and DEX, halted growth of the primary root was observed. Callus formation on the primary root was observed in transformants of 20% of transgenic lines. Formation of green calli was observed in transformants of 60% of these transgenic lines, and transformants of 33% of these lines regenerated shoots from the observed green calli without the application of plant hormones (Table 3).

In transformants of all lines containing the SHOOT REGENERATION-2 vector that were induced with DEX alone, halted growth of the primary root was observed. Callus formation on the primary root was observed in transformants of 28% of transgenic lines. Formation of green calli was observed in transformants of 57% of these transgenic lines, and transformants of all of these lines (100%) regenerated shoots from the observed green calli without the application of plant hormones (Table 3). This represents 16% of all transformants tested (Table 2).

Arabidopsis roots of either transformed line that were not induced with dexamethasone or estradiol never showed any root growth arrest, callus formation or shoot regeneration. Likewise, roots of non-transformed Arabidopsis, induced with 10 μM dexamethasone or estradiol never showed any root growth arrest, callus formation or shoot regeneration (Table 2 and Table 3).

From the plant roots that regenerated shoots, emerging shoots were dissected and transferred to soil where they were observed to form complete plants including roots, able to complete their life cycle and set seeds.

TABLE 2
Effect of induction by dexamethasone (DEX) or 17β-estradiol (EST) or
no induction (none) on Arabidopsis seedlings transformed with construct
SHOOT REGENERATION or SHOOT REGENERATION-2,
or of wild-type (non-transformed) Arabidopsis seedlings.
The developmental effects recorded are interruption of root growth,
callus formation, green callus formation and shoot regeneration.
Numbers of seedlings showing developmental effect are expressed
as percentages of total number of seedlings tested.

| | | root | | | shoot |
|---|---|---|---|---|---|
| Transformation | | growth | | green | regener- |
| vector | induction | arrest | callus | callus | ation |
| SHOOT | DEX + EST | 100 | 100 | 55 | 36 |
| REGENERATION | | | | | |
| SHOOT | DEX | 100 | 82 | 36 | 36 |
| REGENERATION | | | | | |
| SHOOT | none | 0 | 0 | 0 | 0 |
| REGENERATION | | | | | |
| SHOOT | DEX + EST | 100 | 20 | 12 | 4 |
| REGENERATION-2 | | | | | |
| SHOOT | DEX | 100 | 28 | 16 | 16 |
| REGENERATION-2 | | | | | |
| SHOOT | none | 0 | 0 | 0 | 0 |
| REGENERATION-2 | | | | | |
| NON-TRANSFORMED | DEX + EST | 0 | 0 | 0 | 0 |
| CONTROL | | | | | |
| NON-TRANSFORMED | DEX | 0 | 0 | 0 | 0 |
| CONTROL | | | | | |
| NON-TRANSFORMED | none | 0 | 0 | 0 | 0 |
| CONTROL | | | | | |

TABLE 3
Effect of induction by dexamethasone (DEX) or 17β-estradiol (EST) or
no induction (none) on Arabidopsis seedlings transformed with construct
SHOOT REGENERATION or SHOOT REGENERATION-2,
or of wild-type (non-transformed) Arabidopsis seedlings.
The developmental effects recorded are interruption of root growth,
callus formation, green callus formation and shoot regeneration.
The number of seedlings showing root growth arrest and callus
formation is expressed as percentage of total number of seedlings
tested; the numbers showing green callus formation are expressed
as a percentage of the numbers with callus formation; and the
numbers showing shoot regeneration are expressed as a percentage
of the numbers with green callus formation.

| | | root | | | shoot |
|---|---|---|---|---|---|
| Transformation | | growth | | green | regener- |
| vector | induction | arrest | callus | callus | ation |
| SHOOT | DEX + EST | 100 | 100 | 55 | 67 |
| REGENERATION | | | | | |
| SHOOT | DEX | 100 | 82 | 44 | 100 |
| REGENERATION | | | | | |
| SHOOT | none | 0 | 0 | 0 | 0 |
| REGENERATION | | | | | |
| SHOOT | DEX + EST | 100 | 20 | 60 | 33 |
| REGENERATION-2 | | | | | |
| SHOOT | DEX | 100 | 28 | 57 | 100 |
| REGENERATION-2 | | | | | |
| SHOOT | none | 0 | 0 | 0 | 0 |
| REGENERATION-2 | | | | | |
| NON-TRANSFORMED | DEX + EST | 0 | 0 | 0 | 0 |
| CONTROL | | | | | |
| NON-TRANSFORMED | DEX | 0 | 0 | 0 | 0 |
| CONTROL | | | | | |
| NON-TRANSFORMED | none | 0 | 0 | 0 | 0 |
| CONTROL | | | | | |

Excision of the shoots reliably regenerated whole, seed-bearing plants without further induction. Excised shoots were cultured on ½ GM until roots formed naturally. The regenerants could then be grown on soil, where they would bear seeds. This finding was consistent over both construct types, and over several different insertion events.

### Example 2

Induction of Shoot Regeneration in Tomato

Transformation Construct for Tomato

Transformation construct SHOOT REGENERATION from Example 1 above was adapted for tomato transformation by replacing the BASTA herbicide resistance marker by the kanamycin resistance marker nptII (Bevan et al., 1983; SEQ ID NOs: 59 and 64) under control of the Agrobacterium tumefaciens nopaline synthase promoter (NOSp; SEQ ID NO: 33) and the A. tumefaciens octopine synthase terminator (OCST; SEQ ID NO: 60). This construct allowed easy selection of stably transformed tomato tissue on medium containing 50 mg/l kanamycin, for future use (kanamycin-selection was not used in this example). Furthermore, the promoters and terminators to drive the expression of the transcription factors XVE and GVG in the original construct were replaced by the Cauliflower Mosaic Virus 35S promoter (Odell et al., 1985; SEQ ID NO: 61) and the CaMV terminator (T35S; SEQ ID NO: 62). Finally, between the first and the second transcriptional element, a fluorescent reporter gene was placed, consisting of an endoplasmic reticulum (ER)-targeted green fluorescent protein gene (erGFP; SEQ ID NOs: 63 and 65) under control of the same CaMV 35S promoter and CaMV terminator. The resulting plasmid construct was named pKG11051 and was cloned in E. coli and checked by restriction enzyme digestion. Miniprep plasmid DNA was electroporated to Agrobacterium tumefaciens strain GV3101 for plant transformation.

Similarly, transformation construct SHOOT REGENERATION-2 from Example 1 above was adapted by replacing the BASTA marker by nptII, replacing the promoters for XVE and GVG by the CaMV 35S promoter and adding an erGFP fluorescent reporter gene. The resulting plasmid construct was named pKG11052. Miniprep plasmid DNA was electroporated to Agrobacterium tumefaciens strain GV3101 for plant transformation.

Tomato Transformation

Both constructs pKG11051 and pKG11052 were introduced in tomato by Agrobacterium-mediated gene transfer following the method of Koornneef et al. (1986, 1987) with modifications. Approximately 50 tomato seeds were sterilized and germinated on ½MS10 medium for 11 days. Cotyledon explants from the seedlings were dissected and precultured for 24 h on MS20 medium supplemented with 40 μg·l⁻¹ acetosyringone. The explants were submerged in a suspension of Agrobacterium tumefaciens GV3101 carrying pKG11051 or pKG11052 grown overnight in TY medium containing 20 mg·l⁻¹ streptomycin and 50 mg·l⁻¹ spectinomycin, and diluted to OD₆₀₀ 0.138. The explants were blotted dry and cocultivated for two days on plates of MS20 medium with 40 μg·l⁻¹ acetosyringone. The experimental treatment consisted of pre-induction of the transcription factor set by addition of 10 μM ß-estradiol during the cocultivation without the presence of any hormones. The control treatment consisted of addition of 2 mg·l⁻¹ NAA and 1 mg·l⁻¹ BAP to the cocultivation medium as is standard practice for tomato transformation.

After cocultivation, the explants were transferred to medium MS20CV consisting of MS20 medium with 200 mg·l⁻¹ cefotaxim and 200 mg·l⁻¹ vancomycin to suppress further Agrobcaterium growth. In addition, this medium was supplemented with 10 μM dexamethasone to induce the transcription factor genes under control of GVG (experimental treatment) or with 1 mg·l⁻¹ zeatin (plant growth regulator, standard practice standard practice in tomato transformation). In this manner, the effect of the transactivated stem cell genes was compared to the addition of plant growth regulators. The explants were cultivated at 25° C. and 3000 lux (16/8 h photoperiod) in a growth chamber. The explants were subcultured every two weeks onto fresh medium.

Recording of Shoot Regeneration Efficiencies

The experiment was conducted twice in an independent manner. Shoot and callus formation of the explants was recorded 28 days after the start of the experiments (Table 4). The data demonstrate that induction with estradiol and dexamethasone results in hormone-independent shoot formation with efficiencies that are somewhat higher than after conventional shoot induction on plant growth regulators (i.e. first NAA+BAP for two days, then zeatin). This effect is apparent for tomato explants transformed with either construct pKG11051 and pKG11052. Callus is formed on the wounded edges of the explants in all induction treatments.

Control explants without any induction did not show any callus formation nor shoot formation. Non-transformed tomato explants showed the normal shoot induction on medium containing conventional plant growth regulators (i.e. first NAA+BAP for two days, then zeatin), whereas induction by estradiol and dexamethasone in these explants did not result in any shoot formation. This experiment demonstrates that controlled induction of the transcription factor genes present in the gene constructs results in de novo shoot formation without the presence of any growth regulators. The inducers estradiol and dexamethasone alone are not sufficient to induce shoot formation in non-transformed tissues.

TABLE 4
Shoot regeneration efficiencies and callus formation efficiencies
of tomato Moneyberg cotyledon explants transformed with construct
pKG11051 and pKG11052, recorded as the number and percentage
of explants forming shoots and/or callus.

| | | # expl | % expl | # expl | % expl |
|---|---|---|---|---|---|
| | # | with | with | with | with |
| treatment | explants | shoots | shoots | callus | callus |
| control (no induction) | 28 | 0 | 0.0 | 0 | 0.0 |
| pKG1051 | | | | | |
| NAA + BAP, then | 50 | 10 | 20.0 | 50 | 100.0 |
| zeatin | | | | | |
| β-estradiol, | 157 | 45 | 28.0 | (95 *) | 97.9 |
| then dexamethasone | | | | | |
| pKG11052 | | | | | |
| NAA + BAP, then | 58 | 12 | 20.7 | 58 | 100.0 |
| zeatin | | | | | |
| β-estradiol, | 159 | 38 | 23.9 | 156 | 98.1 |
| then dexamethasone | | | | | |
| non-transformed | | | | | |
| NAA + BAP, then | 60 | 21 | 35.0 | 59 | 98.3 |
| zeatin | | | | | |
| β-estradiol, | 61 | 0 | 0.0 | 10 | 16.4 |
| then dexamethasone | | | | | |

* = in this treatment the number of callus-forming explants was recorded per 97 explants tested.

TABLE 5
Description SEQ ID NOs

| SEQ ID NO: | Description |
|---|---|
| 1 | Arabidopsis thaliana SHR amino acid sequence |
| 2 | Arabidopsis thaliana SCR amino acid sequence |
| 3 | Arabidopsis thaliana WOX5 amino acid sequence |
| 4 | Arabidopsis thaliana PLT1 amino acid sequence |
| 5 | Arabidopsis thaliana PLT2 amino acid sequence |
| 6 | Arabidopsis thaliana PLT3 amino acid sequence |
| 7 | Arabidopsis thaliana PLT4 amino acid sequence |
| 8 | Arabidopsis thaliana PLT5 amino acid sequence |
| 9 | Arabidopsis thaliana SHR nucleotide sequence |
| 10 | Arabidopsis thaliana SCR nucleotide sequence |
| 11 | Arabidopsis thaliana WOX5 nucleotide sequence |
| 12 | Arabidopsis thaliana PLT1 nucleotide sequence |
| 13 | Arabidopsis thaliana PLT2 nucleotide sequence |
| 14 | Arabidopsis thaliana PLT3 nucleotide sequence |
| 15 | Arabidopsis thaliana PLT4 nucleotide sequence |
| 16 | Arabidopsis thaliana PLT5 nucleotide sequence |
| 17 | Arabidopsis thaliana RBR amino acid sequence |
| 18 | Arabidopsis thaliana RBR nucleotide sequence |
| 19 | Arabidopsis thaliana PLT7 nucleotide sequence |
| 20 | GVG Transactivator nucleotide sequence |
| 21 | LhGR_Transactivator nucleotide sequence |
| 22 | UAS |
| 23 | RBR miRNA (precursor, as transcribed) |
| 24 | RBR mature miRNA |
| 25 | LacOp |
| 26 | LexAop |
| 27 | XVE transactivator nucleotide sequence |
| 28 | Arabidopsis thaliana WIND1 amino acid sequence |
| 29 | Arabidopsis thaliana WIND1 nucleotide sequence |
| 30 | Arabidopsis thaliana PLT7 amino acid sequence |
| 31 | AlcR Transactivator nucleotide sequence |
| 32 | AlcA |
| 33 | A. tumefaciens nopaline synthase promoter (NOSp) |
| 34 | A. tumefaciens nopaline synthase terminator (NOST) |
| 35 | A. thaliana TRANSLATIONALLY-CONTROLLED |
| | TUMOR PROTEIN 1 promoter (AtTCTP1) |
| 36 | A. thaliana UBIQUITIN10 terminator |
| 37 | G10:90 synthetic promoter |
| 38 | Pisum sativum ribulose-1,5-bisphosphate carboxylase |
| | small subunit terminator (rbcST) |
| 39 | Escherichia coli LexA promoter |
| 40 | 35Smini |
| 41 | Solanum lycopersicum ATPase (SIATPase) terminator |
| 42 | 4xMyc C-tag (4xMyc) nucleotide sequence |
| 43 | 4xMyc C-tag (4xMyc) amino acid sequence |
| 44 | A. thaliana UBIQUITIN3 terminator (AtUBQ3) |
| 45 | 3xFLAG octapeptide C-tag |
| | (3xFLAG) nucleotide sequence |
| 46 | 3xFLAG octapeptide C-tag amino acid sequence |
| 47 | Saccharomyces cerevisiae Upstream Activation Sequence |
| | promoter (UASp) |
| 48 | A. tumefaciens Purified octopine synthase terminator |
| | (AtuOCS) |
| 49 | simian virus 5 C-tag (V5) nucleotide sequence |
| 50 | simian virus 5 C-tag (V5) amino acid sequence |
| 51 | A. tumefaciens mannopine synthase terminator (AtuMas) |
| 52 | bacteriophage T7 gene 10 C-tag (T7) nucleotide sequence |
| 53 | bacteriophage T7 gene 10 C-tag (T7) amino acid sequence |
| 54 | A. thaliana heat shock protein 18.2 terminator (AtHSP) |
| 55 | A. thaliana UBIQUITIN5 terminator (AtUBQ5) |
| 56 | A. thaliana alcohol dehydrogenase terminator (AtADH) |
| 57 | Cauliflower mosaic virus 35S terminator (T35S) |
| 58 | LhGR (transactivator) amino acid sequence |

### REFERENCES

AIDA, M., BEIS, D., HEIDSTRA, R., WILLEMSEN, V., BLILOU, I., GALINHA, C., NUSSAUME, L., NOH, Y. S., AMASINO, R. & SCHERES, B. 2004. The PLETHORA genes mediate patterning of the Arabidopsis root stem cell niche. Cell, 119, 109-20.
AOYAMA, T. & CHUA, N.-H. 1997. A glucocorticoid-mediated transcriptional induction system in transgenic plants. The Plant Journal, 11, 605-612.
BENFEY, P. N., LINSTEAD, P. J., ROBERTS, K., SCHIEFELBEIN, J. W., HAUSER, M. T. & AESCHBACHER, R. A. 1993. Root development in Arabidopsis: four mutants with dramatically altered root morphogenesis. Development, 119, 57-70.
BEVAN, M. W., FLAVELL, R. B., CHILTON, M.-D. (1983) Nature 304: 184-187.
BOUTILIER, K., OFFRINGA, R., SHARMA, V. K., KIEFT, H., OUELLET, T., ZHANG, L., HATTORI, J., LIU, C. M., VAN LAMMEREN, A. A., MIKI, B. L., CUSTERS, J. B. & VAN LOOKEREN CAMPAGNE, M. M. 2002. Ectopic expression of BABY BOOM triggers a conversion from vegetative to embryonic growth. Plant Cell, 14, 1737-49.
BUSK et al. 1997. Plant J. 11: 1285-1295.
CLOUGH S J, BENT A F. Floral dip: a simplified method for Agrobacterium-mediated transformation of Arabidopsis thaliana. The Plant journal: for cell and molecular biology. 1998; 16(6):735-43.
CRAFT et al., 2005. New pOp/LhG4 vectors for stringent glucocorticoid-dependent transgene expression in Arabidopsis. The Plant journal: for cell and molecular biology, 41(6), 899-918.
CRUZ-RAMIREZ, A., DIAZ-TRIVINO, S., WACHSMAN, G., DU, Y., ARTEAGA-VAZQUEZ, M., ZHANG, H., BENJAMINS, R., BLILOU, I., NEEF, A. B., CHANDLER, V. & SCHERES, B. 2013. A SCARECROW-RETINOBLASTOMA protein network controls protective quiescence in the Arabidopsis root stem cell organizer. PLoS Biol, 11, e1001724.
CZECHOWKSI et al. 2005 Genome-wide identification and testing of superior reference genes for transcript normalization in Arabidopsis. Plant Physiol. 139(1):5-17.
DI LAURENZIO, L., WYSOCKA-DILLER, J., MALAMY, J. E., PYSH, L., HELARIUTTA, Y., FRESHOUR, G., HAHN, M. G., FELDMANN, K. A. & BENFEY, P. N. 1996. The SCARECROW gene regulates an asymmetric cell division that is essential for generating the radial organization of the Arabidopsis root. Cell, 86, 423-33.
ENGLER, C., YOULES, M., GRUETZNER, R., EHNERT, T.-M., WERNER, S., JONES, J. D. G., PATRON, N. J. & MARILLONNET, S. 2014. A Golden Gate Modular Cloning Toolbox for Plants. ACS Synth Biol.
FAN, M., XU, C., XU, K. & HU, Y. 2012. LATERAL ORGAN BOUNDARIES DOMAIN transcription factors direct callus formation in Arabidopsis regeneration. 22, 1169-1180.
GALINHA, C., HOFHUIS, H., LUIJTEN, M., WILLEMSEN, V., BLILOU, I., HEIDSTRA, R. & SCHERES, B. 2007. PLETHORA proteins as dose-dependent master regulators of Arabidopsis root development. Nature, 449, 1053-7.
HEIDSTRA and SABATINI. 2014. Plant and animal stem cells: similar yet different. Nature reviews. 15: 301-312.
ISHIGE F, TAKAICHI M, FOSTER R, CHUA N H, OEDA K. 1999. A G-box motif (GCCACGTGCC) tetramer confers high-level constitutive expression in dicot and monocot plants. The Plant Journal. 18(4):443-8.
IWASE, A., MITSUDA, N., KOYAMA, T., HIRATSU, K., KOJIMA, M., ARAI, T., INOUE, Y., SEKI, M., SAKAKIBARA, H., SUGIMOTO, K. & OHME-TAKAGI, M. 2011. The AP2/ERF transcription factor WIND1 controls cell dedifferentiation in Arabidopsis. Curr Biol, 21, 508-14.
IWASE et al. 2015. WIND1-based acquisition of regeneration competency, in Arabidopsis and rapeseed. J Plant Res, 128:389-397.
IWASE et al. 2017. WIND1 Promotes Shoot Regeneration through Transcriptional Activation of ENHANCER OF SHOOT REGENERATION1 in Arabidopsis The Plant Cell, Vol. 29: 54-69.
KAREEM, A., DURGAPRASAD, K., SUGIMOTO, K., DU, Y., PULIANMACKAL, AJAI J., TRIVEDI, ZANKHANA B., ABHAYADEV, PAZHOOR V., PINON, V., MEYEROWITZ, ELLIOT M., SCHERES, B. & PRASAD, K. 2015. PLETHORA Genes Control Regeneration by a Two-Step Mechanism. Current Biology, 25, 1017-1030.
KOORNNEEF, M., HANHART, C., JONGSMA. M., TOMA, I., WEIDE, R, ZABEL, P., HILLE, J. (1986) Plant Science 45: 201-208.
KOORNNEEF, M., HANHART, C. J., MARTINELLI, L. (1987) Theor. Appl. Genet. 74: 633-641.
LEUTWILER, L. S., MEYEROWITZ, E. M., TOBIN, E. M. 1986. Structure and expression of three light-harvesting chlorophyll a/b-binding protein genes in Arabidopsis thaliana. Nucl. Acid Res. 14: 4051-4064.
LINDSEY BE, 3RD, RIVERO L, CALHOUN C S, GROTEWOLD E, BRKLJACIC J. Standardized Method for High-throughput Sterilization of Arabidopsis Seeds. Journal of visualized experiments: JoVE. 2017(128).
MOORE et al, 2006. Transactivated and chemically inducible gene expression in plants. The Plant Journal 45, 651-683.
ODELL et al. 1985. Identification of DNA sequences required for activity of the cauliflower mosaic virus 35S promoter. Nature 313: 810-812.
PELLESCHI et. al. 1999. Plant Mol. Biol. 39:373-380.
PLA et. al. 1993 Plant Mol. Biol. 21:259-266.
PRASAD, K., GRIGG, S. P., BARKOULAS, M., YADAV, R. K., SANCHEZ-PEREZ, G. F., PINON, V., BLILOU, I., HOFHUIS, H., DHONUKSHE, P., GALINHA, C., MAHONEN, A. P., MULLER, W. H., RAMAN, S., VERKLEIJ, A. J., SNEL, B., REDDY, G. V., TSIANTIS, M. & SCHERES, B. 2011. Arabidopsis PLETHORA transcription factors control phyllotaxis. Curr Biol, 21, 1123-8.
ROSSPOPOFF, O., CHELYSHEVA, L., SAFFAR, J., LECORGNE, L., GEY, D., CAILLIEUX, E., COLOT, V., ROUDIER, F., HILSON, P., BERTHOME, R., DA COSTA, M. & RECH, P. 2017. Direct conversion of root primordium into shoot meristem relies on timing of stem cell niche development. Development, 144, 1187-1200.
SARKAR, A. K., LUIJTEN, M., MIYASHIMA, S., LENHARD, M., HASHIMOTO, T., NAKAJIMA, K., SCHERES, B., HEIDSTRA, R. & LAUX, T. 2007. Conserved factors regulate signalling in Arabidopsis thaliana shoot and root stem cell organizers. Nature, 446, 811-4.
SKOOG, F. & MILLER, C. O. 1957. Chemical regulation of growth and organ formation in plant tissues cultured in vitro. Symposia of the Society for Experimental Biology, 11, 118-130.
SUGIMOTO, K., JIAO, Y. & MEYEROWITZ, E. M. 2010. Arabidopsis Regeneration from Multiple Tissues Occurs via a Root Development Pathway. Developmental Cell, 18, 463-471.
TAKAHASHI et al. 1989. Mol. Gen. Genet. 219: 365-372.
TSUWAMOTO, R., YOKOI, S. & TAKAHATA, Y. 2010. Arabidopsis EMBRYOMAKER encoding an AP2 domain transcription factor plays a key role in developmental change from vegetative to embryonic phase. Plant Mol Biol, 73, 481-492.
THOMPSON et al. 1987. The EMBO Journal, 6(9), 2519-2523.
VORST et al. 1990. Plant Mol. Biol. 14: 491-499.
YAMAGUCHI-SHINOZALEI et al. 1993 Mol. Gen. Genet. 236:331-340.
ZUO, J., NIU, Q.-W. & CHUA, N.-H. 2000. An estrogen receptor-based transactivator XVE mediates highly inducible gene expression in transgenic plants. The Plant Journal, 24, 265-273.

1. A method for regenerating a shoot from a plant cell, comprising:
(a) introducing or increasing in the plant cell the expression of a combination of proteins comprising at least:
(i) a WUSCHEL related homeobox 5 (WOX5) protein; and
(ii) a PLETHORA (PLT) protein selected from the group consisting of PLT1, PLT2, PLT3, PLT4, PLT5 and PLT7;
wherein the expression of at least one of the proteins of the combination of proteins is transiently introduced or increased; and
(b) allowing the plant cell to regenerate into the shoot.

2. The method according to claim 1, wherein the PLT protein is PLT1.

3. The method according to claim 1, wherein the combination of proteins further comprises:
(iii) a WOUND INDUCED DEDIFFERENTIATION 1 (WIND1) protein.

4. The method according to claim 1, wherein the combination of proteins further comprises one or more of:
(iv) a SHORT ROOT (SHR) protein;
(v) a SCARECROW (SCR) protein; and
(vi) at least three PLETHORA (PLT) proteins selected from the group consisting of PLT1, PLT2, PLT3, PLT4, PLT5 and PLT7.

5. The method according to claim 1, wherein the combination of proteins comprises at least three selected PLT proteins, which include at least one or more of PLT1, PLT4 and PLT5.

6. The method according to claim 1, wherein (a) further comprises decreasing the expression of an endogenous Retinoblastoma Related (RBR) protein.

7. The method according to claim 1, wherein the expression of all proteins of the combination of proteins is transiently introduced or increased, and wherein optionally the expression of the RBR protein is transiently decreased.

8. The method according to claim 1, wherein the expression of at least one of the proteins of the combination of proteins is transiently introduced or increased by transient activation of their expression and optionally the expression of the RBR protein is transiently decreased by transient activation of the expression of a RBR repressor.

9. The method according to claim 1, wherein:
(i) the amino acid sequence of the SHR protein has at least 60% sequence identity with SEQ ID NO: 1;
(ii) the amino acid sequence of the SCR protein has at least 60% sequence identity with SEQ ID NO: 2;
(iii) the amino acid sequence of the WOX5 protein has at least 60% sequence identity with SEQ ID NO: 3;
(iv) the amino acid sequence of the PLT1, PLT2, PLT3, PLT4, PLT5 and PLT7 proteins have at least 60% sequence identity with SEQ ID NO: 4, SEQ ID NO: 5, SEQ ID NO: 6, SEQ ID NO: 7 SEQ ID NO: 8 and SEQ ID NO: 30, respectively;
(v) the amino acid sequence of the RBR protein has at least 60% sequence identity with SEQ ID NO: 17; and
(vi) the amino acid sequence of the WIND1 protein has at least 60% sequence identity with SEQ ID NO: 28;
and/or wherein:
(a) the SHR protein is encoded by a nucleotide sequence having at least 60% sequence identity with SEQ ID NO: 9;
(b) the SCR protein is encoded by a nucleotide sequence having at least 60% sequence identity with SEQ ID NO: 10;
(c) the WOX5 protein is encoded by a nucleotide sequence having at least 60% sequence identity with SEQ ID NO: 11;
(d) the PLT1, PLT2, PLT3, PLT4, PLT5 and PLT7 proteins are encoded by a nucleotide sequence having at least 60% sequence identity with SEQ ID NO: 12, SEQ ID NO: 13, SEQ ID NO: 14, SEQ ID NO: 15, SEQ ID NO: 16 and SEQ ID NO: 19, respectively;
(e) the RBR protein is encoded by a nucleotide sequence having at least 60% sequence identity with SEQ ID NO: 18; and
(f) the WIND1 protein is encoded by a nucleotide sequence having at least 60% sequence identity with SEQ ID NO: 29.

10. The method according to claim 1, wherein the plant cell is part of a multicellular tissue selected from the group consisting of callus tissue, a plant organ or an explant.

11. The method according to claim 10, wherein the plant organ is a root.

12. The method according to claim 1, wherein the plant cell is obtainable from a plant selected from the group consisting of Arabidopsis, barley, cabbage, canola, cassava, cauliflower, chicory, chrysanthemum, cotton, cucumber, eggplant, grape, hot pepper, lettuce, maize, melon, oilseed rape, potato, pumpkin, rice, rye, sorghum, soybean, squash, sugar cane, sugar beet, sunflower, sweet pepper, tomato, water melon, wheat, and zucchini.

13. The method according to claim 1, wherein the method further comprises (c) forming a plant or plant part from the regenerated shoot.

14. A composition, comprising:
(i) a first nucleic acid molecule comprising a nucleotide sequence encoding a WOX5 protein operably linked to an inducible promoter; and
(ii) a second nucleic acid molecule comprising a nucleotide sequence encoding a PLT protein selected from the group consisting of PLT1, PLT2, PLT3, PLT4, PLT5 and PLT7, operably linked to an inducible promoter.

15. The composition according to claim 1, wherein the PLT protein is PLT1.

16. A nucleic acid construct, comprising:
(i) a first nucleic acid molecule comprising a nucleotide sequence encoding a WOX5 protein operably linked to an inducible promoter; and
(ii) a second nucleic acid molecule comprising a nucleotide sequence encoding a PLT protein selected from the group consisting of PLT1, PLT2, PLT3, PLT4, PLT5 and PLT7, operably linked to an inducible promoter.

17. The nucleic acid construct according to claim 16, further comprising nucleotide sequence encoding a transactivator, optionally operably linked to a promoter, wherein the transactivator upon binding an inducer, activates the inducible promoter.

18. The nucleic acid construct according to claim 17, wherein the transactivator is encoded by a nucleotide sequence having
i) at least 60% sequence identity with SEQ ID NO: 21 and wherein the transactivator is capable of binding to dexamethasone corticoid or a derivative thereof; or
ii) at least 60% sequence identity with SEQ ID NO: 27 and wherein the transactivator is capable of binding to β-estradiol or a derivative thereof.

19. A plant cell, comprising the first and second nucleic acid molecule as defined in claim 14.

20. A shoot, plant or plant part obtainable by the method according to claim 1.
